SEQUENCE LISTING (51 sequences)

SEQ ID NO: 1 - protein, 1001 aa, synthetic construct
METDTLLLWVLLLWVPGSTGDYKDEYPYDVPDYAGSAHAGRTGYDNREIVMKYIHYKLSQRGYEWDAGDDAEENRTEAPEGTESEVVHRALRDAGDDFERRYRRDFAEMSSQLHLTPDTARQRFETVVEELFRDGVNWGRIVAFFEFGGVMCVESVNREMSPLVDNIAEWMTEYLNRHLHTWIQDNGGWDAFVELYGPSMRGGGGSGGGGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 2 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 3 - protein, 158 aa, synthetic construct
DLGKKLLEAARAGQDDEVRILVANGADVNAYFGTTPLHLAAAHGRLEIVEVLLKNGADVNAQDVWGITPLHLAAYNGHLEIVEVLLKYGADVNAHDTRGWTPLHLAAINGHLEIVEVLLKNVADVNAQDRSGKTPFDLAIDNGNEDIAEVLQKAAKLN

SEQ ID NO: 4 - protein, 483 aa, synthetic construct
MGSHHHHHHGSGSENLYFQGSGGSDLGKKLLEAARAGQDDEVRILVANGADVNAYFGTTPLHLAAAHGRLEIVEVLLKNGADVNAQDVWGITPLHLAAYNGHLEIVEVLLKYGADVNAHDTRGWTPLHLAAINGHLEIVEVLLKNVADVNAQDRSGKTPFDLAIDNGNEDIAEVLQKAAKLNSGSGSGKPGQASGSELARKLLEASTKLQRLNIRLAEALLEAIARLQELNLELVYLAVELTDPKRIRDEIKEVKDKSKEIIRRAEKEIDDAAKESEKILEEAREAISGSGSELAKLLLKAIAETQDLNLRAAKAFLEAAAKLQELNIRAVELLVKLTDPATIREALEHAKRRSKEIIDEAERAIRAAKRESERIIEEARRLIEKGSGSGSELARELLRAHAQLQRLNLELLRELLRALAQLQELNLDLLRLASELTDEIWIAQELRRIGDEFNAYYADAERLIREAAAASEKISREAERLIR

SEQ ID NO: 5 - protein, 245 aa, synthetic construct
MGSHHHHHHGSGSENLYFQGSGGSDEARKAIARVKRESKRIVEDAERLIREAAAASEKISREAERLIRGGGSGSGSGSGKPGQASGSDLGKKLLEAARAGQDDEVRILVANGADVNAYFGTTPLHLAAAHGRLEIVEVLLKNGADVNAQDVWGITPLHLAAYNGHLEIVEVLLKYGADVNAHDTRGWTPLHLAAINGHLEIVEVLLKNVADVNAQDRSGKTPFDLAIDNGNEDIAEVLQKAAKLN

SEQ ID NO: 6 - protein, 1156 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL

SEQ ID NO: 7 - protein, 314 aa, Homo sapiens; identical to SEQ ID NO: 2

SEQ ID NO: 8 - protein, 1156 aa, Homo sapiens; identical to SEQ ID NO: 6

SEQ ID NO: 9 - protein, 1161 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSGLWVLGGSIAQGFRSVAAVEAQTFYFDKQLKKSKNVIGSLDAYNGQE

SEQ ID NO: 10 - protein, 1121 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSGGHSEPH

SEQ ID NO: 11 - protein, 1154 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSSKTQKTSLIGSYTVKGTGLGSHSCLFLKPCLLPGYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSGGHSEPH

SEQ ID NO: 12 - protein, 357 aa, synthetic construct
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNADPAFLYKVVDIQHSGGRSSLEGPRFEGKPIPNPLLGLDSTRTG

SEQ ID NO: 13 - protein, 1200 aa, synthetic construct
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNLLNPAFLYKVVDIQHSGGRSSLEGPRFEGKPIPNPLLGLDSTRTG

SEQ ID NO: 14 - protein, 368 aa, synthetic construct
METDTLLLWVLLLWVPGSTGDGSHHHHHHGSGSENLYFQGSGGSDEARKAIARVKRESKRIVEDAERLIREAAAASEKISREAERLIRGGGSGSGSGSGKPGQASGSELQMTQSPSSLSASVGDRVTITCRASQSISSYLNWYQQKPGQPPKLLIYWASTRESGVPDRFSGSESGTNYTLTISSLQPEDFATYFCQQSDSLPITFGQGTRLDIQGSTSGSGKPGSGEGSGEVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSSAPTKAPDVFPL

SEQ ID NO: 15 - protein, 365 aa, synthetic construct
METDTLLLWVLLLWVPGSTGDGSHHHHHHGSGSENLYFQGSGGSDEAIARVKRESKRIVEDAERLIREAAAASEKISREAERLIRGGGSGSGSGSGKPGQASGSELQMTQSPSSLSASVGDRVTITCRASQSISSYLNWYQQKPGQPPKLLIYWASTRESGVPDRFSGSESGTNYTLTISSLQPEDFATYFCQQSDSLPITFGQGTRLDIQGSTSGSGKPGSGEGSGEVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSSAPTKAPDVFPL

SEQ ID NO: 16 - protein, 606 aa, synthetic construct
METDTLLLWVLLLWVPGSTGDGSHHHHHHGSGSENLYFQGSGGSELQMTQSPSSLSASVGDRVTITCRASQSISSYLNWYQQKPGQPPKLLIYWASTRESGVPDRFSGSESGTNYTLTISSLQPEDFATYFCQQSDSLPITFGQGTRLDIQGSTSGSGKPGSGEGSGEVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSSAPTKAPDVFPLSGSGSGKPGQASGSELARKLLEASTKLQRLNIRLAEALLEAIARLQELNLELVYLAVELTDPKRIRDEIKEVKDKSKEIIRRAEKEIDDAAKESEKILEEAREAISGSGSELAKLLLKAIAETQDLNLRAAKAFLEAAAKLQELNIRAVELLVKLTDPATIREALEHAKRRSKEIIDEAERAIRAAKRESERIIEEARRLIEKGSGSGSELARELLRAHAQLQRLNLELLRELLRALAQLQELNLDLLRLASELTDEIWIAQELRRIGDEFNAYYADAERLIREAAAASEKISREAERLIR

SEQ ID NO: 17 - protein, 606 aa, synthetic construct
METDTLLLWVLLLWVPGSTGDGSHHHHHHGSGSENLYFQGSGGSELQMTQSPSSLSASVGDRVTITCRASQSISSYLNWYQQKPGQPPKLLIYWASTRESGVPDRFSGSESGTNYTLTISSLQPEDFATYFCQQSDSLPITFGQGTRLDIQGSTSGSGKPGSGEGSGEVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSSAPTKAPDVFPLSGSGSGKPGQASGSELARKLLEASTKLQRLNIRLAEALLEAIARLQELNLELVYLAVELTDPKRIRDEIKEVKDKSKEIIRRAEKEIDDAAKESEKILEEAREAISGSGSELAKLLLKAIAETQDLNLRAAKAFLEAAAKLQELNIRAVELLVKLTDPATIREALEHAKRRSKEIIDEAERAIRAAKRESERIIEEARRLIEKGSGSGSELARELLRAHAQLQRLNLELLRELLRALAQLQELNLDLLRLASELTDEIWIAQELRRIGDEFNAYYADAERLIREAAAASEKISREAERLAR

SEQ ID NO: 18 - protein, 347 aa, synthetic construct
METDTLLLWVLLLWVPGSTGDGSHHHHHHGSGSENLYFQGSGGSDEARKAIARVKRESKRIVEDAERLIREAAAASEKISREAERLIRGGGSGSGSGSGKPGQASGSQVQLQESGPGLVKPSETLSLTCTVSGYSITSDYAWNWIRQFPGNKLEWMGYITYSGSTYYNPSLKSRVTISVDTSKNQFSLKLSSVTAADTATYYCVSGTYYFDYWGQGTTLTVSSGSTSGSGKPGSGEGSGDIVMTQSPSSLSASVGDRVTITCRASQSVSSDVAWFQQKPGKSPKPLIYYASNRYSGVPSRFSGSGSGTDFTLTISSLQAEDFAVYFCGQDYTSPRTFGGGTKLEIKR

SEQ ID NO: 19 - protein, 344 aa, synthetic construct
METDTLLLWVLLLWVPGSTGDGSHHHHHHGSGSENLYFQGSGGSDEAIARVKRESKRIVEDAERLIREAAAASEKISREAERLIRGGGSGSGSGSGKPGQASGSQVQLQESGPGLVKPSETLSLTCTVSGYSITSDYAWNWIRQFPGNKLEWMGYITYSGSTYYNPSLKSRVTISVDTSKNQFSLKLSSVTAADTATYYCVSGTYYFDYWGQGTTLTVSSGSTSGSGKPGSGEGSGDIVMTQSPSSLSASVGDRVTITCRASQSVSSDVAWFQQKPGKSPKPLIYYASNRYSGVPSRFSGSGSGTDFTLTISSLQAEDFAVYFCGQDYTSPRTFGGGTKLEIKR

SEQ ID NO: 20 - protein, 585 aa, synthetic construct
METDTLLLWVLLLWVPGSTGDGSHHHHHHGSGSENLYFQGSGGSQVQLQESGPGLVKPSETLSLTCTVSGYSITSDYAWNWIRQFPGNKLEWMGYITYSGSTYYNPSLKSRVTISVDTSKNQFSLKLSSVTAADTATYYCVSGTYYFDYWGQGTTLTVSSGSTSGSGKPGSGEGSGDIVMTQSPSSLSASVGDRVTITCRASQSVSSDVAWFQQKPGKSPKPLIYYASNRYSGVPSRFSGSGSGTDFTLTISSLQAEDFAVYFCGQDYTSPRTFGGGTKLEIKRSGSGSGKPGQASGSELARKLLEASTKLQRLNIRLAEALLEAIARLQELNLELVYLAVELTDPKRIRDEIKEVKDKSKEIIRRAEKEIDDAAKESEKILEEAREAISGSGSELAKLLLKAIAETQDLNLRAAKAFLEAAAKLQELNIRAVELLVKLTDPATIREALEHAKRRSKEIIDEAERAIRAAKRESERIIEEARRLIEKGSGSGSELARELLRAHAQLQRLNLELLRELLRALAQLQELNLDLLRLASELTDEIWIAQELRRIGDEFNAYYADAERLIREAAAASEKISREAERLIR

SEQ ID NO: 21 - protein, 585 aa, synthetic construct
METDTLLLWVLLLWVPGSTGDGSHHHHHHGSGSENLYFQGSGGSQVQLQESGPGLVKPSETLSLTCTVSGYSITSDYAWNWIRQFPGNKLEWMGYITYSGSTYYNPSLKSRVTISVDTSKNQFSLKLSSVTAADTATYYCVSGTYYFDYWGQGTTLTVSSGSTSGSGKPGSGEGSGDIVMTQSPSSLSASVGDRVTITCRASQSVSSDVAWFQQKPGKSPKPLIYYASNRYSGVPSRFSGSGSGTDFTLTISSLQAEDFAVYFCGQDYTSPRTFGGGTKLEIKRSGSGSGKPGQASGSELARKLLEASTKLQRLNIRLAEALLEAIARLQELNLELVYLAVELTDPKRIRDEIKEVKDKSKEIIRRAEKEIDDAAKESEKILEEAREAISGSGSELAKLLLKAIAETQDLNLRAAKAFLEAAAKLQELNIRAVELLVKLTDPATIREALEHAKRRSKEIIDEAERAIRAAKRESERIIEEARRLIEKGSGSGSELARELLRAHAQLQRLNLELLRELLRALAQLQELNLDLLRLASELTDEIWIAQELRRIGDEFNAYYADAERLIREAAAASEKISREAERLAR

SEQ ID NO: 22 - protein, 423 aa, synthetic construct
MLLLVTSLLLCELPHPAFLLIPELQMTQSPSSLSASVGDRVTITCRASQSISSYLNWYQQKPGQPPKLLIYWASTRESGVPDRFSGSESGTNYTLTISSLQPEDFATYFCQQSDSLPITFGQGTRLDIQGSTSGSGKPGSGEGSGEVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSSAPTKAPDVFPLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 23 - protein, 461 aa, synthetic construct
MLLLVTSLLLCELPHPAFLLIPQVQLQESGPGLVKPSETLSLTCTVSGYSITSDYAWNWIRQFPGNKLEWMGYITYSGSTYYNPSLKSRVTISVDTSKNQFSLKLSSVTAADTATYYCVSGTYYFDYWGQGTTLTVSSGSTSGSGKPGSGEGSGDIVMTQSPSSLSASVGDRVTITCRASQSVSSDVAWFQQKPGKSPKPLIYYASNRYSGVPSRFSGSGSGTDFTLTISSLQAEDFAVYFCGQDYTSPRTFGGGTKLEIKRALSNSIMYFSHFVPVFLPAKPTTTPAPRPPTPAPTIASQPLSLRPEASRPAAGGAVHTRGLDFACDIYIWAPLAGTCGVLLLSLVITRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 459 aa, synthetic construct
MALPVTALLLPLALLLHAARPQVQLQESGPGLVKPSETLSLTCTVSGYSITSDYAWNWIRQFPGNKLEWMGYITYSGSTYYNPSLKSRVTISVDTSKNQFSLKLSSVTAADTATYYCVSGTYYFDYWGQGTTLTVSSGGGGSGGGGSGGGGSDIVMTQSPSSLSASVGDRVTITCRASQSVSSDVAWFQQKPGKSPKPLIYYASNRYSGVPSRFSGSGSGTDFTLTISSLQAEDFAVYFCGQDYTSPRTFGGGTKLEIKRALSNSIMYFSHFVPVFLPAKPTTTPAPRPPTPAPTIASQPLSLRPEASRPAAGGAVHTRGLDFACDIYIWAPLAGTCGVLLLSLVITRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 477 aa, synthetic construct
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQSYSTPRGTFGQGTKVEIKGGGGSGGGGSGGGGSEVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARENVRPYYDFWSGYYSEYYYYGMDVWGQGTTVTVSSAALSNSIMYFSHFVPVFLPAKPTTTPAPRPPTPAPTIASQPLSLRPEASRPAAGGAVHTRGLDFACDIYIWAPLAGTCGVLLLSLVITRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 467 aa, synthetic construct
MALPVTALLLPLALLLHAARPQVQLQESGPGLVKPSETLSLTCTVSGYSITSDYAWNWIRQFPGNKLEWMGYITYSGSTYYNPSLKSRVTISVDTSKNQFSLKLSSVTAADTATYYCVSGTYYFDYWGQGTTLTVSSGGGGSGGGGSGGGGSDIVMTQSPSSLSASVGDRVTITCRASQSVSSDVAWFQQKPGKSPKPLIYYASNRYSGVPSRFSGSGSGTDFTLTISSLQAEDFAVYFCGQDYTSPRTFGGGTKLEIKRDYKDDDDKALSNSIMYFSHFVPVFLPAKPTTTPAPRPPTPAPTIASQPLSLRPEASRPAAGGAVHTRGLDFACDIYIWAPLAGTCGVLLLSLVITRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 138 aa, synthetic construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSSAPTKAPDVFPL

SEQ ID NO: 28 - protein, 107 aa, synthetic construct
ELQMTQSPSSLSASVGDRVTITCRASQSISSYLNWYQQKPGQPPKLLIYWASTRESGVPDRFSGSESGTNYTLTISSLQPEDFATYFCQQSDSLPITFGQGTRLDIQ

SEQ ID NO: 29 - protein, 116 aa, synthetic construct
QVQLQESGPGLVKPSETLSLTCTVSGYSITSDYAWNWIRQFPGNKLEWMGYITYSGSTYYNPSLKSRVTISVDTSKNQFSLKLSSVTAADTATYYCVSGTYYFDYWGQGTTLTVSS

SEQ ID NO: 30 - protein, 108 aa, synthetic construct
DIVMTQSPSSLSASVGDRVTITCRASQSVSSDVAWFQQKPGKSPKPLIYYASNRYSGVPSRFSGSGSGTDFTLTISSLQAEDFAVYFCGQDYTSPRTFGGGTKLEIKR

SEQ ID NO: 31 - protein, 134 aa, synthetic construct
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARENVRPYYDFWSGYYSEYYYYGMDVWGQGTTVTVSSA

SEQ ID NO: 32 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPRGTFGQGTKVEI

SEQ ID NO: 33 - protein, 5 aa, synthetic construct
SYGMH

SEQ ID NO: 34 - protein, 17 aa, synthetic construct
VISYDGSNKYYADSVKG

SEQ ID NO: 35 - protein, 20 aa, synthetic construct
KDMGWGSGWRPYYYYGMDVW

SEQ ID NO: 36 - protein, 11 aa, synthetic construct
RASQSISSYLN

SEQ ID NO: 37 - protein, 7 aa, synthetic construct
WASTRES

SEQ ID NO: 38 - protein, 10 aa, synthetic construct
QQSDSLPITF

SEQ ID NO: 39 - protein, 8 aa, synthetic construct
GYSITSDY

SEQ ID NO: 40 - skipped

SEQ ID NO: 41 - protein, 12 aa, synthetic construct
CVSGTYYFDYWG

SEQ ID NO: 42 - protein, 9 aa, synthetic construct
ASQSVSSDV

SEQ ID NO: 43 - skipped

SEQ ID NO: 44 - protein, 8 aa, synthetic construct
QDYTSPRT

SEQ ID NO: 45 - protein, 5 aa, synthetic construct
SYAMH

SEQ ID NO: 46 - protein, 16 aa, synthetic construct
VISYDGSNKYYADSVK

SEQ ID NO: 47 - protein, 24 aa, synthetic construct
ENVRPYYDFWSGYYSEYYYYGMDV

SEQ ID NO: 48 - protein, 11 aa, synthetic construct
RASQSISSYLN

SEQ ID NO: 49 - protein, 7 aa, synthetic construct
AASSLQS

SEQ ID NO: 50 - protein, 10 aa, synthetic construct
QQSYSTPRGT

SEQ ID NO: 51 - protein, 6 aa, synthetic construct
HHHHHH

(19) United States
(12) Patent Application Publication
(10) Pub. No.: US 2025/0051475 A1
(43) Pub. Date: Feb. 13, 2025

(54) BISPECIFIC MOLECULES TO TARGET THE FIRST CELL

(71) Applicant: The Trustees of Columbia University in the City of New York, New York, NY (US)

(72) Inventors: Azra RAZA, New York, NY (US); Abdullah M. ALI, Tenafly, NJ (US)

(21) Appl. No.: 18/719,725

(22) PCT Filed: Dec. 30, 2022
(86) PCT No.: PCT/US2022/082628

§ 371 Date: Jun. 13, 2024

Related U.S. Application Data
(60) Provisional application No. 63/295,681, filed on Dec. 31, 2021.

(51) Int. Cl. C07K 16/30; A61K 47/68; C07K 16/28; G01N 33/574

(52) U.S. Cl. CPC C07K 16/30; A61K 47/6851; C07K 16/2896; G01N 33/574; C07K 2317/31; C07K 2317/565

(57) ABSTRACT

The subject matter described herein relates to a method of treating cancer in a subject in need thereof using a bispecific molecule recognizing two antigen markers of different tissue lineages on the surface of The First Cell.

This application claims the benefit and priority to U.S. Provisional Patent Application No. 63/295,681, filed on Dec. 31, 2021, the contents of which is hereby incorporated by reference in its entirety.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosure of these publications in their entireties are hereby incorporated by reference into this application in order to more fully describe the state of the art as known to those skilled therein as of the date of the invention described herein.

### BACKGROUND

Cancer represents a group of conditions characterized by abnormal cell growth. Cancerous cells have the potential to spread and invade other organs of the body. The most common symptoms of cancer include a lump, abnormal bleeding, prolonged cough, and unexplained weight loss. There are also cancers of the blood which do not form a cell mass. Over 100 types of cancers can develop within the human body and most of them are incurable.

### SUMMARY OF THE INVENTION

In certain aspects the subject matter described herein provides a bispecific molecule comprising at least two antigen binding regions, wherein each antigen binding region binds a different antigen on The First Cell (TFC) of a cancer.

In some embodiments, the first antigen is a marker of epithelial cell lineage. In some embodiments, the marker of epithelial cell lineage is any one of the markers in FIG. 2. In some embodiments, the marker of epithelial cell lineage is epithelial cellular adhesion molecule (EpCAM). In some embodiments, EpCAM comprises SEQ ID NO: 7 or SEQ ID NO: 12. In some embodiments, the second antigen is a marker of macrophage cell lineage. In some embodiments, the marker of macrophage cell lineage is any one of the markers in FIG. 1. In some embodiments, the marker of macrophage cell lineage is CD163. In some embodiments, CD163 comprises SEQ ID NO: 8, SEQ ID NO: 9, SEQ ID NO:10, SEQ ID NO: 11, or SEQ ID NO: 13.

In some embodiments, the cancer comprises a solid tumor. In some embodiments, the cancer is breast cancer, brain cancer, gastrointestinal cancer, pancreatic cancer, kidney cancer, liver cancer, lung cancer, thymic carcinoma, ovarian cancer, prostate cancer, or endometrial cancer. In some embodiments, the gastrointestinal cancer is stomach cancer or colorectal cancer.

In some embodiments, the cancer comprises a liquid cancer. In some embodiments, the liquid cancer is leukemia, lymphoma, or myeloma. In some embodiments, the liquid cancer is acute myeloid leukemia (AML). In some embodiments, the liquid cancer is B-cell malignancy. In some embodiments, the liquid cancer is myeloid neoplasm, myelodysplastic syndrome (MDS), myeloproliferative neoplasm (MPN), MDS/MPN overlap syndrome, acute myeloid leukemia or chronic myeloid leukemia.

In some embodiments, the first antigen binding region comprises a light chain variable (VL) region and a heavy chain variable (VH) region, wherein the VL region comprises a light chain CDR1 (CDRL1) of SEQ ID NO: 36, a light chain CDR2 (CDRL2) of SEQ ID NO: 37, a light chain CDR3 (CDRL3) of SEQ ID NO: 38 and the VH region comprises a heavy chain CDR1 of SEQ ID NO: 33, a heavy chain CDR2 of SEQ ID NO: 34, and a heavy chain CDR3 of SEQ ID NO: 35. In some embodiments, the second antigen binding region comprises a light chain variable (VL) region and a heavy chain variable (VH) region, wherein the VL region comprises a light chain CDR1 (CDRL1) of SEQ ID NO: 42, a light chain CDR2 (CDRL2) of SEQ ID NO: 43, a light chain CDR3 (CDRL3) of SEQ ID NO: 44 and the VH region comprises a heavy chain CDR1 of SEQ ID NO: 39, a heavy chain CDR2 of SEQ ID NO: 40, and a heavy chain CDR3 of SEQ ID NO: 41. In some embodiments, the second antigen binding region comprises a light chain variable (VL) region and a heavy chain variable (VH) region, wherein the VL region comprises a light chain CDR1 (CDRL1) of SEQ ID NO: 48, a light chain CDR2 (CDRL2) of SEQ ID NO: 49, a light chain CDR3 (CDRL3) of SEQ ID NO: 50 and the VH region comprises a heavy chain CDR1 of SEQ ID NO: 45, a heavy chain CDR2 of SEQ ID NO: 46, and a heavy chain CDR3 of SEQ ID NO: 47.

In some embodiments, the first antigen binding region comprises a light chain variable (VL) region of SEQ ID NO: 28 and a heavy chain variable (VH) region of SEQ ID NO:27. In some embodiments, the second antigen binding region comprises a light chain variable (VL) region of SEQ ID NO: 30 and a heavy chain variable (VH) region of SEQ ID NO: 29. In some embodiments, the second antigen binding region comprises a light chain variable (VL) region of SEQ ID NO: 32 and a heavy chain variable (VH) region of SEQ ID NO: 31.

In some embodiments, the first antigen and the second antigen are markers of macrophage cell lineage. In some embodiments, the first antigen is CD117, CD34, or CD123 and the second antigen is CD163. In some embodiments, TFC is a metastatic TFC.

In some embodiments, the bispecific molecule is a bispecific antibody or antigen binding fragment thereof. In some embodiments, the bispecific antibody is conjugated to a drug. In some embodiments, the drug is a toxin. In some embodiments, the drug is a chemotherapy agent. In some embodiments, the bispecific molecule comprises bi-nanobodies, BiTE, tandAbs, DARTs, DART-Fc, DARPin, scFv, scFv-HAS-scFV, and DNL-Fab3. In some embodiments, the bispecific molecule is a bispecific chimeric antigen receptor (CAR). In some embodiments, the bispecific molecule is a Co-LOCKR comprising a first polypeptide and a second polypeptide, wherein the first polypeptide and the second polypeptide each bind a different antigen on TFC of a cancer.

In some embodiments, the first polypeptide comprises SEQ ID NO: 14, SEQ ID NO: 15, SEQ ID NO: 18, or SEQ ID NO: 19. In some embodiments, the second polypeptide comprises SEQ ID NO: 16, SEQ ID NO: 17, SEQ ID NO: 20, or SEQ ID NO: 21.

In some embodiments, the bispecific CAR is a synNotch CAR. In some embodiments, the bispecific molecule is a Co-LOCKR comprising a first polypeptide, a second polypeptide, and a third polypeptide, wherein the first polypeptide and the second polypeptide each bind a different antigen on TFC of a cancer, wherein the third polypeptide binds to a CAR, and wherein the third polypeptide is operably linked to the first polypeptide or the second polypeptide.

In some embodiments, the bispecific molecule comprises a split-CAR-T system comprising a chimeric antigen receptor (CAR) module and a chimeric costimulatory receptor (CCR) module, wherein the CAR module comprises a polypeptide comprising a first antigen binding region and CD3z signaling domain, wherein the CCR module comprises a polypeptide comprising a second antigen binding region and two or more co-stimulatory domains, and wherein the CAR module and the CCR module each bind a different antigen on TFC of a cancer. In some embodiments, the split-CAR-T system comprises one or more of the polypeptide sequences in Table 4.

In certain aspects the subject matter described herein provides a pharmaceutical composition comprising a bispecific molecule according to any embodiment described herein.

In certain aspects the subject matter described herein provides a polynucleotide encoding a bispecific molecule according to any embodiment described herein.

In certain aspects the subject matter described herein provides a vector comprising a polynucleotide according to any embodiment described herein.

In certain aspects the subject matter described herein provides a virus comprising a polynucleotide according to any embodiment described herein.

In certain aspects the subject matter described herein provides a genetically engineered cell comprising a bispecific molecule according to any embodiment described herein.

In certain aspects the subject matter described herein provides a genetically engineered cell comprising a polynucleotide according to any embodiment described herein.

In certain aspects the subject matter described herein provides a method of treating or preventing cancer in a subject in need thereof, the method comprising administering to the subject a bispecific molecule comprising at least two antigen binding regions, wherein each antigen binding region binds a different antigen on The First Cell (TFC) of a cancer.

In some embodiments, the first antigen is a marker of epithelial cell lineage. In some embodiments, the marker of epithelial cell lineage is any one of the markers in FIG. 2. In some embodiments, the marker of epithelial cell lineage is epithelial cellular adhesion molecule (EpCAM). In some embodiments, EpCAM comprises SEQ ID NO: 7 or SEQ ID NO: 12.

In some embodiments, the second antigen is a marker of macrophage cell lineage. In some embodiments, the marker of macrophage cell lineage is any one of the markers in FIG. 1. In some embodiments, the marker of macrophage cell lineage is CD163. In some embodiments, CD163 comprises SEQ ID NO: 8, SEQ ID NO: 9, SEQ ID NO: 10, SEQ ID NO: 11, or SEQ ID NO: 13.

In some embodiments, the cancer comprises a solid tumor. In some embodiments, the cancer is breast cancer, brain tumor, gastrointestinal, pancreatic cancer, kidney cancer, liver cancer, lung cancer, thymic carcinoma, ovarian cancer, prostate cancer, or endometrial cancer. In some embodiments, the gastrointestinal cancer is stomach cancer or colorectal cancer.

In some embodiments, the cancer is a liquid cancer. In some embodiments, the liquid cancer is leukemia, lymphoma, or myeloma. In some embodiments, the liquid cancer is acute myeloid leukemia (AML). In some embodiments, the liquid cancer is B-cell malignancy. In some embodiments, the liquid cancer is myeloid neoplasm, myelodysplastic syndrome (MDS), myeloproliferative neoplasm (MPN), MDS/MPN overlap syndrome, acute myeloid leukemia or chronic myeloid leukemia.

In some embodiments, the first antigen binding region comprises a light chain variable (VL) region and a heavy chain variable (VH) region, wherein the VL region comprises a light chain CDR1 (CDRL1) of SEQ ID NO: 36, a light chain CDR2 (CDRL2) of SEQ ID NO: 37, a light chain CDR3 (CDRL3) of SEQ ID NO: 38 and the VH region comprises a heavy chain CDR1 of SEQ ID NO: 33, a heavy chain CDR2 of SEQ ID NO: 34, and a heavy chain CDR3 of SEQ ID NO: 35. In some embodiments, the second antigen binding region comprises a light chain variable (VL) region and a heavy chain variable (VH) region, wherein the VL region comprises a light chain CDR1 (CDRL1) of SEQ ID NO: 42, a light chain CDR2 (CDRL2) of SEQ ID NO: 43, a light chain CDR3 (CDRL3) of SEQ ID NO: 44 and the VH region comprises a heavy chain CDR1 of SEQ ID NO: 39, a heavy chain CDR2 of SEQ ID NO: 40, and a heavy chain CDR3 of SEQ ID NO: 41. In some embodiments, the second antigen binding region comprises a light chain variable (VL) region and a heavy chain variable (VH) region, wherein the VL region comprises a light chain CDR1 (CDRL1) of SEQ ID NO: 48, a light chain CDR2 (CDRL2) of SEQ ID NO: 49, a light chain CDR3 (CDRL3) of SEQ ID NO: 50 and the VH region comprises a heavy chain CDR1 of SEQ ID NO: 45, a heavy chain CDR2 of SEQ ID NO: 46, and a heavy chain CDR3 of SEQ ID NO: 47.

In some embodiments, the first antigen binding region comprises a light chain variable (VL) region of SEQ ID NO: 28 and a heavy chain variable (VH) region of SEQ ID NO:27. In some embodiments, the second antigen binding region comprises a light chain variable (VL) region of SEQ ID NO: 30 and a heavy chain variable (VH) region of SEQ ID NO: 29. In some embodiments, the second antigen binding region comprises a light chain variable (VL) region of SEQ ID NO: 32 and a heavy chain variable (VH) region of SEQ ID NO: 31.

In some embodiments, the first antigen and the second antigen are markers of macrophage cell lineage. In some embodiments, the first antigen is CD117, CD34, or CD123 and the second antigen is CD163.

In some embodiments, TFC is a metastatic TFC. In some embodiments, the bispecific molecule is a bispecific antibody or antigen binding fragment thereof.

In some embodiments, the bispecific antibody is conjugated to a drug. In some embodiments, the drug is a toxin. In some embodiments, the drug is a chemotherapy agent.

In some embodiments, the bispecific molecule comprises bi-nanobodies, BiTE, tandAbs, DARTs, DART-Fc, DARPin, scFv, scFv-HAS-scFV, and DNL-Fab3. In some embodiments, the bispecific molecule is a bispecific chimeric antigen receptor (CAR). In some embodiments, the bispecific molecule is a Co-LOCKR comprising a first polypeptide and a second polypeptide, wherein the first polypeptide and the second polypeptide each bind a different antigen on TFC of a cancer. In some embodiments, the first polypeptide comprises SEQ ID NO: 14, SEQ ID NO: 15, SEQ ID NO: 18, or SEQ ID NO: 19. In some embodiments, the second polypeptide comprises SEQ ID NO: 16, SEQ ID NO: 17, SEQ ID NO: 20, or SEQ ID NO: 21.

In some embodiments, the bispecific CAR is a synNotch CAR. In some embodiments, the bispecific molecule is a Co-LOCKR comprising a first polypeptide, a second polypeptide, and a third polypeptide, wherein the first polypeptide and the second polypeptide each bind a different antigen on TFC of a cancer, wherein the third polypeptide binds to the bispecific CAR, and wherein the third polypeptide is operably linked to the first polypeptide or the second polypeptide.

In some embodiments, the bispecific molecule comprises a split-CAR-T system comprising a chimeric antigen receptor (CAR) module and a chimeric costimulatory receptor (CCR) module, wherein the CAR module comprises a polypeptide comprising a first antigen binding region and CD3z signaling domain, wherein the CCR module comprises a polypeptide comprising a second antigen binding region and two or more co-stimulatory domains, and wherein the CAR module and the CCR module each bind a different antigen on TFC of a cancer. In some embodiments, the split-CAR-T system comprises one or more of the polypeptide sequences in Table 4.

In certain aspects the subject matter described herein provides an engineered cell expressing at least one marker of epithelial cell lineage and at least one marker of macrophage cell lineage.

In some embodiments, the at least one marker of epithelial cell lineage is any one of the markers in FIG. 2. In some embodiments, the at least one marker of epithelial cell lineage is epithelial cellular adhesion molecule (EpCAM). In some embodiments, EpCAM comprises SEQ ID NO: 7 or SEQ ID NO: 12.

In some embodiments, the at least one marker of macrophage cell lineage is any one of the markers in FIG. 1. In some embodiments, the at least one marker of macrophage cell lineage is CD163. In some embodiments, CD163 comprises SEQ ID NO: 8, SEQ ID NO: 9, SEQ ID NO: 10, SEQ ID NO: 11, or SEQ ID NO: 13.

In certain aspects the subject matter described herein provides a method of diagnosing cancer, wherein the method comprises detecting a cell expressing at least one marker of epithelial cell lineage and at least one marker of macrophage cell lineage.

In some embodiments, the at least one marker of epithelial cell lineage is any one of the markers in FIG. 2. In some embodiments, the at least one marker of epithelial cell lineage is epithelial cellular adhesion molecule (EpCAM). In some embodiments, EpCAM comprises SEQ ID NO: 7.

In some embodiments, the at least one marker of macrophage cell lineage is any one of the markers in FIG. 1. In some embodiments, the at least one marker of macrophage cell lineage is CD163. In some embodiments, CD163 comprises SEQ ID NO: 8, SEQ ID NO: 9, SEQ ID NO: 10, or SEQ ID NO: 11.

In some embodiments, the detecting comprises an assay wherein a bispecific molecule binds to the at least one marker of epithelial cell lineage and the at least one marker of macrophage cell lineage.

In some embodiments, the cancer comprises a solid tumor. In some embodiments, the cancer is breast cancer, brain cancer, gastrointestinal cancer, pancreatic cancer, kidney cancer, liver cancer, lung cancer, thymic carcinoma, ovarian cancer, prostate cancer, or endometrial cancer. In some embodiments, the gastrointestinal cancer is stomach cancer or colorectal cancer. In some embodiments, the cancer is a liquid cancer. In some embodiments, the liquid cancer is leukemia, lymphoma, or myeloma. In some embodiments, the liquid cancer is acute myeloid leukemia (AML). In some embodiments, the liquid cancer is B-cell malignancy. In some embodiments, the liquid cancer is myeloid neoplasm, myelodysplastic syndrome (MDS), myeloproliferative neoplasm (MPN), MDS/MPN overlap syndrome, acute myeloid leukemia or chronic myeloid leukemia.

### BRIEF DESCRIPTION OF FIGURES

One or more of the figures is presented in color. To conform to the requirements for PCT patent applications, many of the figures presented herein are black and white representations of images originally created in color.

FIG. 1 shows antigens that exhibit predominant or exclusive macrophage expression. In the last two columns, a “+” signifies that expression is present, a “−” signifies that expression is absent, a blank signifies that the expression unknown.

FIG. 2 shows antigens that exhibit predominant or exclusive epithelial expression. In the last two columns, a “+” signifies that expression is present, a “−” signifies that expression is absent, a blank signifies that the expression unknown.

FIGS. 3A-B show one embodiment of a Co-LOCKR. FIG. 3A shows s conformational change of a Co-LOCKR. FIG. 3B shows a Co-LOCKR recruiting a CAR-T cell.

FIG. 4 shows separation of cells by size where large cells are selected for.

FIGS. 5A-D show a schematic representation of cells with no expression of lineage specific antigen (LSA) (A), expression of each LSA alone (B and C) or both antigens together (D). The cells with no expression or expression of individual LSA expression are used as control to show the specificity of various modalities tested on the target.

FIGS. 6A-C show a flow cytometry analysis of expression of either CD163 (A) or EpCAM (B) or both CD163 and EpCAM (C). The left panels show isotype controls and the right panels show staining with antibodies recognizing EpCAM (Clone 9C4, PerCP/Cyanine5.5 mouse monoclonal IgG2; Cat #324213 Biolegend) or CD163 (Clone RM3/1, PE conjugated mouse monoclonal IgG2; Cat #326506 Biolegend).

FIGS. 7A-C show a schematic representation of binding of Cage and Key respectively to cells expressing either antigen A (panel A) or antigen B (panel B). The Cage is in closed conformation and sequesters the latch when Key is not co-localized with Cage (panel A) but the latch is released from Cage when the Key and Cage binds and co-localize with antigen A and B on the same cells (panel C).

FIGS. 8A-B show a schematic representation of various components of the Co-LOCKR components Cage and Key proteins (panel A) or variants of Cage and Key proteins (panel B). The antigen binding domains to A or B proteins are made in both Cage and Key combinations to identify optimal combination.

FIGS. 9A-B show expression and purification of Co-LOCKR proteins. A) Coomassie G250 stained gel. B) Immunoblot using anti-HIs6 antibody. 1) PageRuler™ Plus Prestained Protein Ladder. 2) His6_TEV_EpCAM-ScFV_Cage (˜64 kDa). 3) His6_TEV_Key_EpCAM-ScFV (˜38 kDa). 4) His6_TEV_Key_N3_EpCAM-ScFV (˜37 kDa). 5) PageRuler™ Plus Prestained Protein Ladder. 6) His6_TEV_CD163-ScFV_Cage (˜62 kDa). 7) His6_TEV_Key_CD163-ScFV (˜35.8 kDa). 8) His6_TEV_CD163-ScFV_Cage_I287A (˜62 kDa). 9. His6_TEV_Key_N3_CD163-ScFV (˜35.5 kDa).

FIG. 10 shows a schematic representation of an engagement of a Split-CAR-T cell expressing CAR module and CCR module. When a split-CAR-T engages a target cell expressing both antigens A and B, then it gets both activated and co-stimulated resulting in the eradication of cells expressing A and B. Engagement of split-CAR-T with cells expressing either A or B results in sub-optimal activation.

FIG. 11 shows a schematic representation of various components of CAR and CCR modules of Split-CAR-T system. The antigen binding domains to A or B proteins are made in both CAR and CCR combinations to identify optimal combination.

### DETAILED DESCRIPTION OF THE INVENTION

In certain aspects the subject matter described herein provides a bispecific molecule comprising at least two antigen binding regions, wherein each antigen binding region binds a different antigen on The First Cell (TFC) of a cancer.

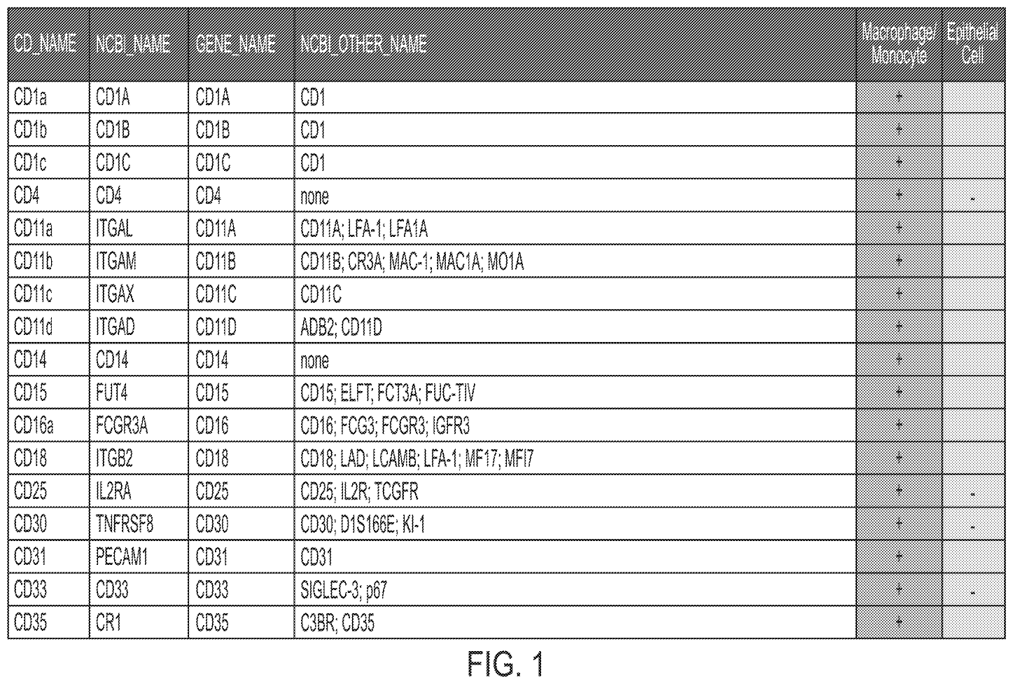

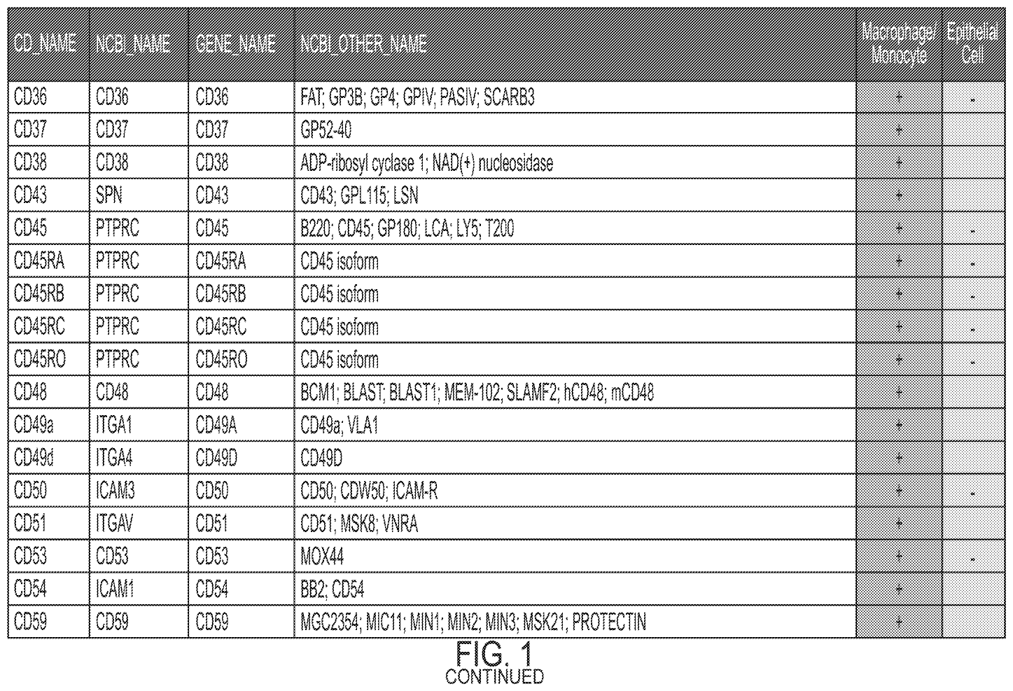

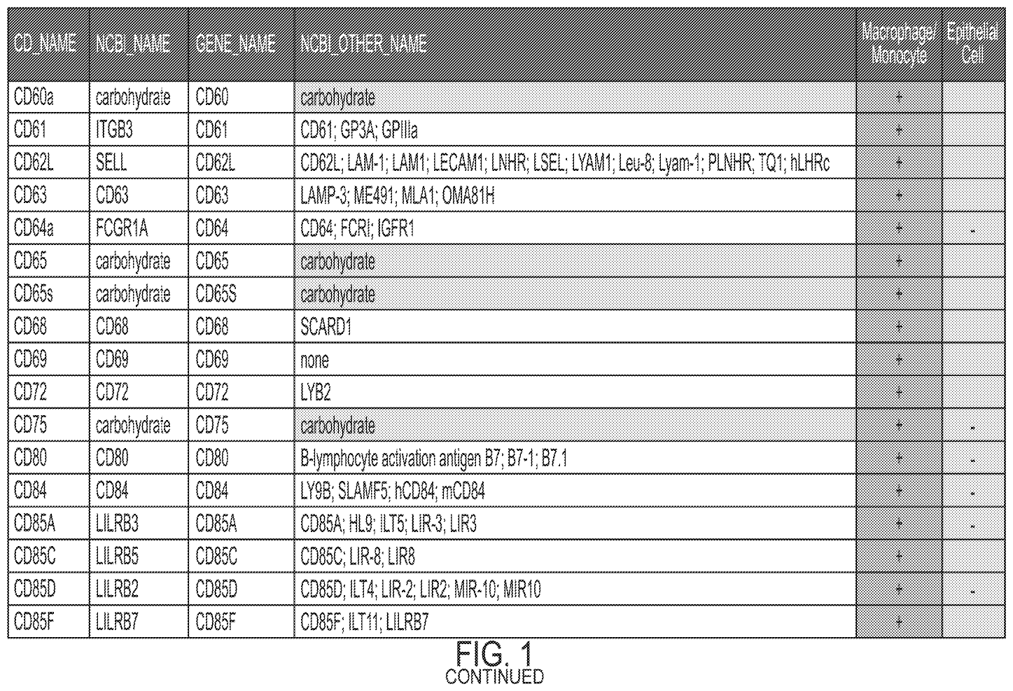

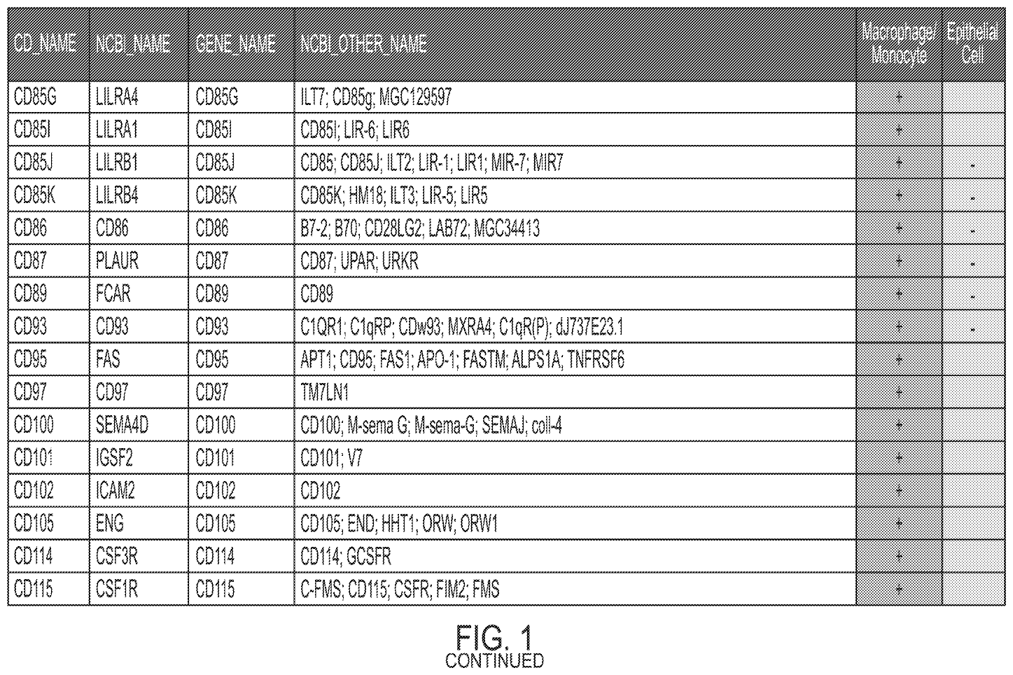

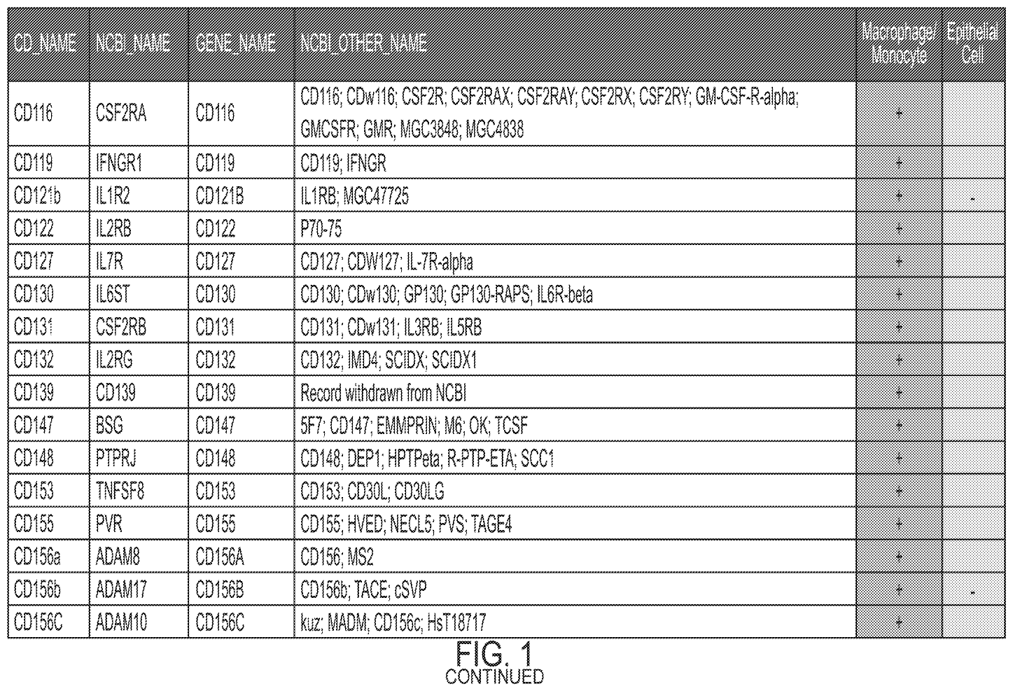

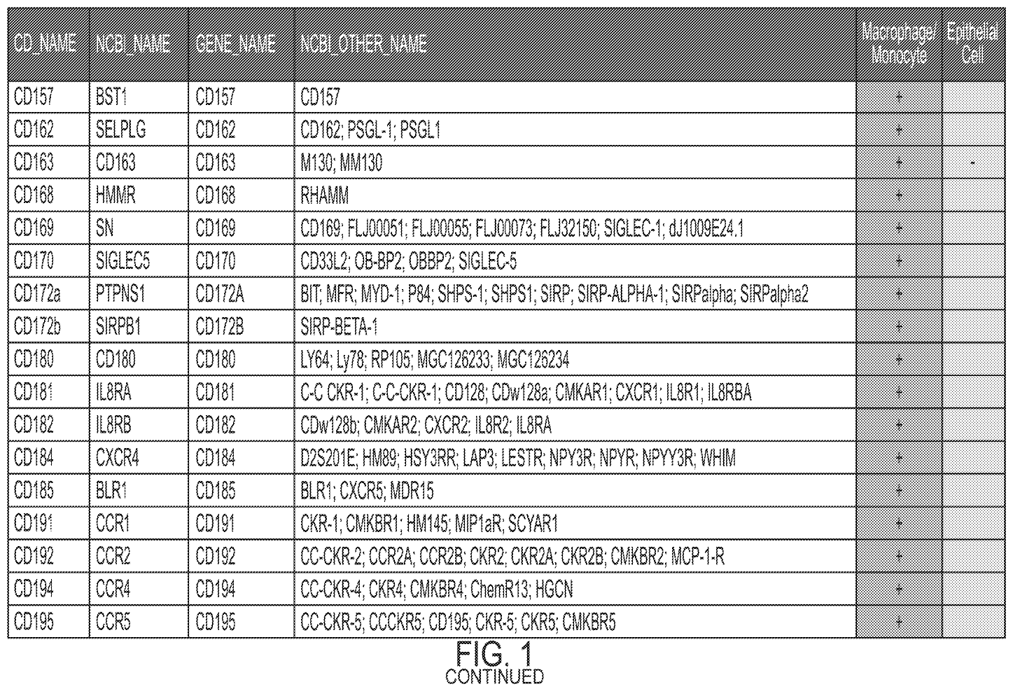

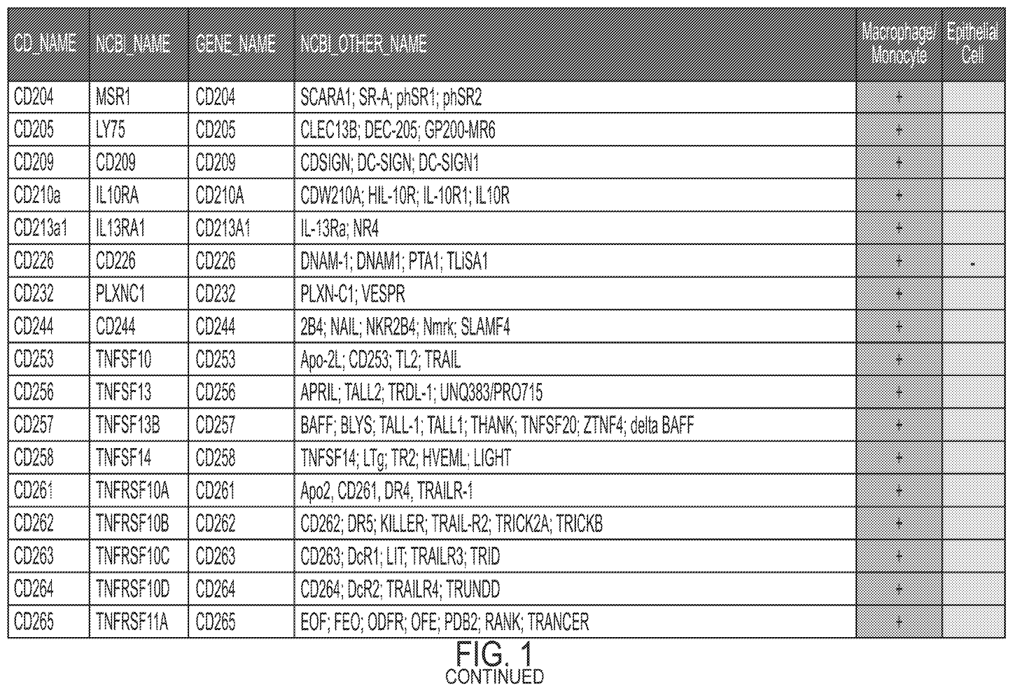

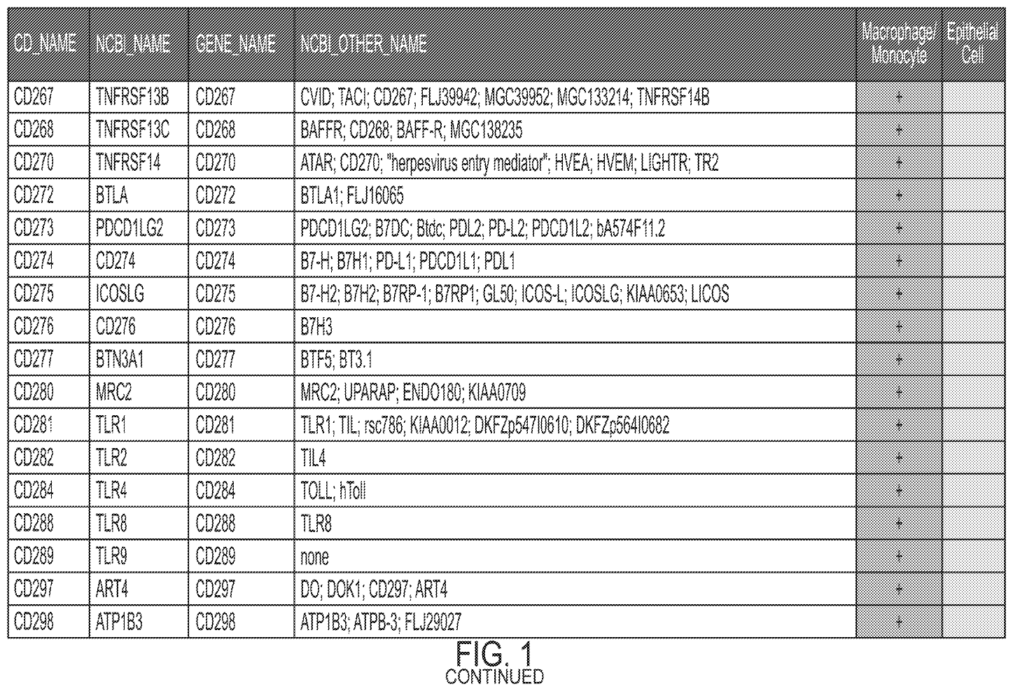

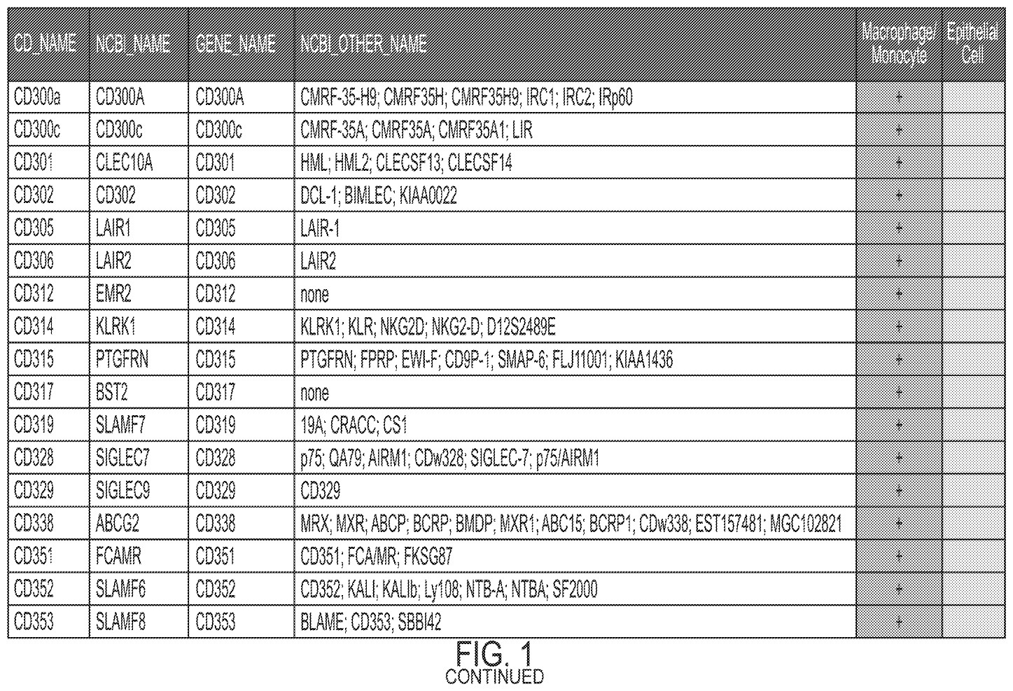

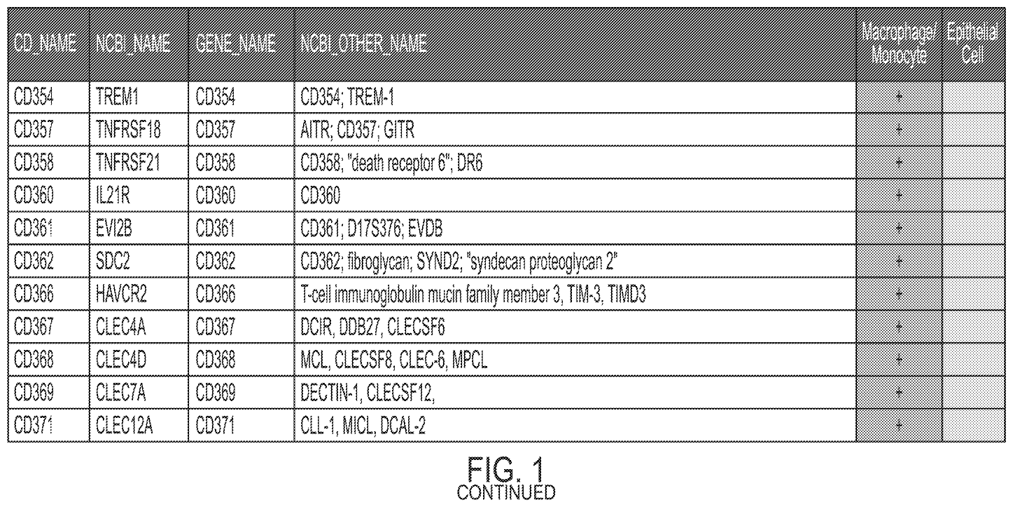

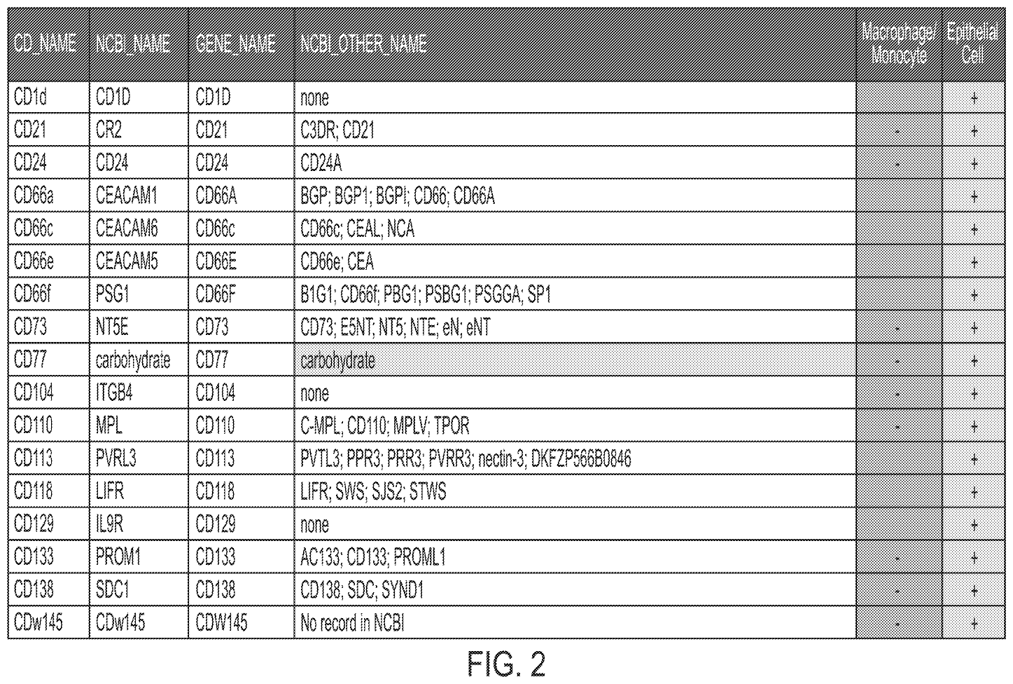

In some embodiments, the first antigen is a marker of epithelial cell lineage. In some embodiments, the marker of epithelial cell lineage is any one of the markers in FIG. 2. In some embodiments, the marker of epithelial cell lineage is epithelial cellular adhesion molecule (EpCAM). In some embodiments, EpCAM comprises SEQ ID NO: 7 or SEQ ID NO: 12. In some embodiments, the second antigen is a marker of macrophage cell lineage. In some embodiments, the marker of macrophage cell lineage is any one of the markers in FIG. 1. In some embodiments, the marker of macrophage cell lineage is CD163. In some embodiments, CD163 comprises SEQ ID NO: 8, SEQ ID NO: 9, SEQ ID NO:10, SEQ ID NO: 11, or SEQ ID NO: 13.

In some embodiments, the cancer comprises a solid tumor. In some embodiments, the cancer is breast cancer, brain cancer, gastrointestinal cancer, pancreatic cancer, kidney cancer, liver cancer, lung cancer, thymic carcinoma, ovarian cancer, prostate cancer, or endometrial cancer. In some embodiments, the gastrointestinal cancer is stomach cancer or colorectal cancer.

In some embodiments, the cancer comprises a liquid cancer. In some embodiments, the liquid cancer is leukemia, lymphoma, or myeloma. In some embodiments, the liquid cancer is acute myeloid leukemia (AML). In some embodiments, the liquid cancer is B-cell malignancy. In some embodiments, the liquid cancer is myeloid neoplasm, myelodysplastic syndrome (MDS), myeloproliferative neoplasm (MPN), MDS/MPN overlap syndrome, acute myeloid leukemia or chronic myeloid leukemia.

In some embodiments, the first antigen binding region comprises a light chain variable (VL) region and a heavy chain variable (VH) region, wherein the VL region comprises a light chain CDR1 (CDRL1) of SEQ ID NO: 36, a light chain CDR2 (CDRL2) of SEQ ID NO: 37, a light chain CDR3 (CDRL3) of SEQ ID NO: 38 and the VH region comprises a heavy chain CDR1 of SEQ ID NO: 33, a heavy chain CDR2 of SEQ ID NO: 34, and a heavy chain CDR3 of SEQ ID NO: 35. In some embodiments, the second antigen binding region comprises a light chain variable (VL) region and a heavy chain variable (VH) region, wherein the VL region comprises a light chain CDR1 (CDRL1) of SEQ ID NO: 42, a light chain CDR2 (CDRL2) of SEQ ID NO: 43, a light chain CDR3 (CDRL3) of SEQ ID NO: 44 and the VH region comprises a heavy chain CDR1 of SEQ ID NO: 39, a heavy chain CDR2 of SEQ ID NO: 40, and a heavy chain CDR3 of SEQ ID NO: 41. In some embodiments, the second antigen binding region comprises a light chain variable (VL) region and a heavy chain variable (VH) region, wherein the VL region comprises a light chain CDR1 (CDRL1) of SEQ ID NO: 48, a light chain CDR2 (CDRL2) of SEQ ID NO: 49, a light chain CDR3 (CDRL3) of SEQ ID NO: 50 and the VH region comprises a heavy chain CDR1 of SEQ ID NO: 45, a heavy chain CDR2 of SEQ ID NO: 46, and a heavy chain CDR3 of SEQ ID NO: 47.

In some embodiments, the first antigen binding region comprises a light chain variable (VL) region of SEQ ID NO: 28 and a heavy chain variable (VH) region of SEQ ID NO:27. In some embodiments, the second antigen binding region comprises a light chain variable (VL) region of SEQ ID NO: 30 and a heavy chain variable (VH) region of SEQ ID NO: 29. In some embodiments, the second antigen binding region comprises a light chain variable (VL) region of SEQ ID NO: 32 and a heavy chain variable (VH) region of SEQ ID NO: 31.

In some embodiments, the first antigen and the second antigen are markers of macrophage cell lineage. In some embodiments, the first antigen is CD117, CD34, or CD123 and the second antigen is CD163. In some embodiments, TFC is a metastatic TFC.

In some embodiments, the bispecific molecule is a bispecific antibody or antigen binding fragment thereof. In some embodiments, the bispecific antibody is conjugated to drug. In some embodiments, the drug is a toxin. In some embodiments, the drug is a chemotherapy agent. In some embodiments, the bispecific molecule comprises bi-nanobodies, BiTE, tandAbs, DARTs, DART-Fc, DARPin, scFv, scFv-HAS-scFV, and DNL-Fab3. In some embodiments, the bispecific molecule is a bispecific chimeric antigen receptor (CAR). In some embodiments, the bispecific molecule is a Co-LOCKR comprising a first polypeptide and a second polypeptide, wherein the first polypeptide and the second polypeptide each bind a different antigen on TFC of a cancer.

In some embodiments, the first polypeptide comprises SEQ ID NO: 14, SEQ ID NO: 15, SEQ ID NO: 18, or SEQ ID NO: 19. In some embodiments, the second polypeptide comprises SEQ ID NO: 16, SEQ ID NO: 17, SEQ ID NO: 20, or SEQ ID NO: 21.

In some embodiments, the bispecific CAR is a synNotch CAR. In some embodiments, the bispecific molecule is a Co-LOCKR comprising a first polypeptide, a second polypeptide, and a third polypeptide, wherein the first polypeptide and the second polypeptide each bind a different antigen on TFC of a cancer, wherein the third polypeptide binds to a CAR, and wherein the third polypeptide is operably linked to the first polypeptide or the second polypeptide.

In some embodiments, the bispecific molecule comprises a split-CAR-T system comprising a chimeric antigen receptor (CAR) module and a chimeric costimulatory receptor (CCR) module, wherein the CAR module comprises a polypeptide comprising a first antigen binding region and CD3z signaling domain, wherein the CCR module comprises a polypeptide comprising a second antigen binding region and two or more co-stimulatory domains, and wherein the CAR module and the CCR module each bind a different antigen on TFC of a cancer. In some embodiments, the split-CAR-T system comprises one or more of the polypeptide sequences in Table 4.

In certain aspects the subject matter described herein provides a pharmaceutical composition comprising a bispecific molecule according to any embodiment described herein.

In certain aspects the subject matter described herein provides a polynucleotide encoding a bispecific molecule according to any embodiment described herein.

In certain aspects the subject matter described herein provides a vector comprising a polynucleotide according to any embodiment described herein.

In certain aspects the subject matter described herein provides a virus comprising a polynucleotide according to any embodiment described herein.

In certain aspects the subject matter described herein provides a genetically engineered cell comprising a bispecific molecule according to any embodiment described herein.

In certain aspects the subject matter described herein provides a genetically engineered cell comprising a polynucleotide according to any embodiment described herein.

In certain aspects the subject matter described herein provides a method of treating or preventing cancer in a subject in need thereof, the method comprising administering to the subject a bispecific molecule comprising at least two antigen binding regions, wherein each antigen binding region binds a different antigen on The First Cell (TFC) of a cancer.

In some embodiments, the first antigen is a marker of epithelial cell lineage. In some embodiments, the marker of epithelial cell lineage is any one of the markers in FIG. 2. In some embodiments, the marker of epithelial cell lineage is epithelial cellular adhesion molecule (EpCAM). In some embodiments, EpCAM comprises SEQ ID NO: 7 or SEQ ID NO: 12. In some embodiments, the second antigen is a marker of macrophage cell lineage. In some embodiments, the marker of macrophage cell lineage is any one of the markers in FIG. 1. In some embodiments, the marker of macrophage cell lineage is CD163. In some embodiments, CD163 comprises SEQ ID NO: 8, SEQ ID NO: 9, SEQ ID NO: 10, SEQ ID NO: 11, or SEQ ID NO: 13.

In some embodiments, the cancer comprises a solid tumor. In some embodiments, the cancer is breast cancer, brain tumor, gastrointestinal, pancreatic cancer, kidney cancer, liver cancer, lung cancer, thymic carcinoma, ovarian cancer, prostate cancer, or endometrial cancer. In some embodiments, the gastrointestinal cancer is stomach cancer or colorectal cancer.

In some embodiments, the cancer is a liquid cancer. In some embodiments, the liquid cancer is leukemia, lymphoma, or myeloma. In some embodiments, the liquid cancer is acute myeloid leukemia (AML). In some embodiments, the liquid cancer is B-cell malignancy. In some embodiments, the liquid cancer is myeloid neoplasm, myelodysplastic syndrome (MDS), myeloproliferative neoplasm (MPN), MDS/MPN overlap syndrome, acute myeloid leukemia or chronic myeloid leukemia.

In some embodiments, the first antigen binding region comprises a light chain variable (VL) region and a heavy chain variable (VH) region, wherein the VL region comprises a light chain CDR1 (CDRL1) of SEQ ID NO: 36, a light chain CDR2 (CDRL2) of SEQ ID NO: 37, a light chain CDR3 (CDRL3) of SEQ ID NO: 38 and the VH region comprises a heavy chain CDR1 of SEQ ID NO: 33, a heavy chain CDR2 of SEQ ID NO: 34, and a heavy chain CDR3 of SEQ ID NO: 35. In some embodiments, the second antigen binding region comprises a light chain variable (VL) region and a heavy chain variable (VH) region, wherein the VL region comprises a light chain CDR1 (CDRL1) of SEQ ID NO: 42, a light chain CDR2 (CDRL2) of SEQ ID NO: 43, a light chain CDR3 (CDRL3) of SEQ ID NO: 44 and the VH region comprises a heavy chain CDR1 of SEQ ID NO: 39, a heavy chain CDR2 of SEQ ID NO: 40, and a heavy chain CDR3 of SEQ ID NO: 41. In some embodiments, the second antigen binding region comprises a light chain variable (VL) region and a heavy chain variable (VH) region, wherein the VL region comprises a light chain CDR1 (CDRL1) of SEQ ID NO: 48, a light chain CDR2 (CDRL2) of SEQ ID NO: 49, a light chain CDR3 (CDRL3) of SEQ ID NO: 50 and the VH region comprises a heavy chain CDR1 of SEQ ID NO: 45, a heavy chain CDR2 of SEQ ID NO: 46, and a heavy chain CDR3 of SEQ ID NO: 47.

In some embodiments, the first antigen binding region comprises a light chain variable (VL) region of SEQ ID NO: 28 and a heavy chain variable (VH) region of SEQ ID NO:27. In some embodiments, the second antigen binding region comprises a light chain variable (VL) region of SEQ ID NO: 30 and a heavy chain variable (VH) region of SEQ ID NO: 29. In some embodiments, the second antigen binding region comprises a light chain variable (VL) region of SEQ ID NO: 32 and a heavy chain variable (VH) region of SEQ ID NO: 31.

In some embodiments, the first antigen and the second antigen are markers of macrophage cell lineage. In some embodiments, the first antigen is CD117, CD34, or CD123 and the second antigen is CD163.

In some embodiments, TFC is a metastatic TFC. In some embodiments, the bispecific molecule is a bispecific antibody or antigen binding fragment thereof.

In some embodiments, the bispecific antibody is conjugated to a drug. In some embodiments, the drug is a toxin. In some embodiments, the drug is a chemotherapy agent.

In some embodiments, the bispecific molecule comprises bi-nanobodies, BiTE, tandAbs, DARTs, DART-Fc, DARPin, scFv, scFv-HAS-scFV, and DNL-Fab3. In some embodiments, the bispecific molecule is a bispecific chimeric antigen receptor (CAR). In some embodiments, the bispecific molecule is a Co-LOCKR comprising a first polypeptide and a second polypeptide, wherein the first polypeptide and the second polypeptide each bind a different antigen on TFC of a cancer. In some embodiments, the first polypeptide comprises SEQ ID NO: 14, SEQ ID NO: 15, SEQ ID NO: 18, or SEQ ID NO: 19. In some embodiments, the second polypeptide comprises SEQ ID NO: 16, SEQ ID NO: 17, SEQ ID NO: 20, or SEQ ID NO: 21.

In some embodiments, the bispecific CAR is a synNotch CAR. In some embodiments, the bispecific molecule is a Co-LOCKR comprising a first polypeptide, a second polypeptide, and a third polypeptide, wherein the first polypeptide and the second polypeptide each bind a different antigen on TFC of a cancer, wherein the third polypeptide binds to the bispecific CAR, and wherein the third polypeptide is operably linked to the first polypeptide or the second polypeptide.

In some embodiments, the bispecific molecule comprises a split-CAR-T system comprising a chimeric antigen receptor (CAR) module and a chimeric costimulatory receptor (CCR) module, wherein the CAR module comprises a polypeptide comprising a first antigen binding region and CD3z signaling domain, wherein the CCR module comprises a polypeptide comprising a second antigen binding region and two or more co-stimulatory domains, and wherein the CAR module and the CCR module each bind a different antigen on TFC of a cancer. In some embodiments, the split-CAR-T system comprises one or more of the polypeptide sequences in Table 4.

In certain aspects the subject matter described herein provides an engineered cell expressing at least one marker of epithelial cell lineage and at least one marker of macrophage cell lineage.

In some embodiments, the at least one marker of epithelial cell lineage is any one of the markers in FIG. 2. In some embodiments, the at least one marker of epithelial cell lineage is epithelial cellular adhesion molecule (EpCAM). In some embodiments, EpCAM comprises SEQ ID NO: 7 or SEQ ID NO: 12.

In some embodiments, the at least one marker of macrophage cell lineage is any one of the markers in FIG. 1. In some embodiments, the at least one marker of macrophage cell lineage is CD163. In some embodiments, CD163 comprises SEQ ID NO: 8, SEQ ID NO: 9, SEQ ID NO: 10, SEQ ID NO: 11, or SEQ ID NO: 13.

In certain aspects the subject matter described herein provides a method of diagnosing cancer, wherein the method comprises detecting a cell expressing at least one marker of epithelial cell lineage and at least one marker of macrophage cell lineage.

In some embodiments, the at least one marker of epithelial cell lineage is any one of the markers in FIG. 2. In some embodiments, the at least one marker of epithelial cell lineage is epithelial cellular adhesion molecule (EpCAM). In some embodiments, EpCAM comprises SEQ ID NO: 7.

In some embodiments, the at least one marker of macrophage cell lineage is any one of the markers in FIG. 1. In some embodiments, the at least one marker of macrophage cell lineage is CD163. In some embodiments, CD163 comprises SEQ ID NO: 8, SEQ ID NO: 9, SEQ ID NO: 10, or SEQ ID NO: 11.

In some embodiments, the detecting comprises an assay wherein a bispecific molecule binds to the at least one marker of epithelial cell lineage and the at least one marker of macrophage cell lineage.

In some embodiments, the cancer comprises a solid tumor. In some embodiments, the cancer is breast cancer, brain cancer, gastrointestinal cancer, pancreatic cancer, kidney cancer, liver cancer, lung cancer, thymic carcinoma, ovarian cancer, prostate cancer, or endometrial cancer. In some embodiments, the gastrointestinal cancer is stomach cancer or colorectal cancer. In some embodiments, the cancer is a liquid cancer. In some embodiments, the liquid cancer is leukemia, lymphoma, or myeloma. In some embodiments, the liquid cancer is acute myeloid leukemia (AML). In some embodiments, the liquid cancer is B-cell malignancy. In some embodiments, the liquid cancer is myeloid neoplasm, myelodysplastic syndrome (MDS), myeloproliferative neoplasm (MPN), MDS/MPN overlap syndrome, acute myeloid leukemia or chronic myeloid leukemia.

Specific targeting of cancer cells has been a major challenge in the cancer field.¹ Most common approaches to cancer treatment such as chemotherapy and radiotherapy are indiscriminate and target both cancer cells and normal (non-cancer or healthy) cells, causing patients to experience a multitude of painful side effects. Targeted approaches to killing cancer cells, such as immunotherapeutic approaches including antibodies and chimeric antigen therapy (CAR-T) have the ability to eliminate cells expressing one or more pre-determined antigens with precision. However, the major challenge is to identify antigens, specific to cancer cells, that can be targeted.^{1,2} This challenge is due to lack of a single unique antigen that is present only on cancer cells and not on normal cells. Strategies to differentiate cancer cells include targeting two or more antigens.¹

In some embodiments, the subject matter disclosed herein relates to the discovery that most cancers do not arise spontaneously. Stress to an organ or tissue can cause the stressed cells to develop heroic survival strategies. In some embodiments, one of these cell survival strategies includes fusion with a blood derived macrophage.³⁻⁶ This hybrid tissue cell and macrophage is called The First Cell (TFC) and it gives rise to cancerous growths. Thus, cancer does not necessarily begin in one cell but it can begin in two cells.

In some embodiments, this TFC undergoes genomic re-organization and re-engineering with multiple consequences including:

In some embodiments, the TFC has the ability to evade the immune system—because the TFC expresses macrophage markers, it can evade the immune system.^{4,5,7-13}

In some embodiments, the TFC is a hybrid, retaining properties of both tissues of origin. Being one-part macrophage, the TFC can travel all over the body with impunity and can be significantly associated with metastasis.^{4,5,7-13}

In some embodiments, the TFC can be visually identified. In some embodiments, the TFC appears as a giant polyploid cell. In some embodiments, the TFC can be observed as a giant polyploid cell in 100% of solid tumors. In some embodiments, the TFC can be observed as a giant polyploid cell in Myelodysplastic syndrome (MDS) cases. In some embodiments, the TFC can be observed as a giant polyploid cell in acute myeloid leukemia (AML) cases.⁷

In solid tumors, the TFC can express at least one marker of the epithelial tissue from which it is derived and at least one marker for macrophages^{5,14}. In some embodiments, the epithelial tissue marker is epithelial cellular adhesion molecule (EpCAM). EpCAM is found in epithelial cells lining the surfaces and cavities of the body. EpCAM can span the membrane of the epithelial cells and it is important for cell adhesion. In some embodiments, the macrophage marker is CD163, a scavenger receptor for haptoglobin-hemoglobin complexes.

In some embodiments, the TFC is distinct from other tumor cells. In some embodiments, the TFC is distinct from circulating tumor cells (CTCs).⁷

In some embodiments, the TFC is also called CAML (Cancer Associated Macrophage Like cell) or PACC (Poly-Aneuploid Cancer cell) or PGCC (Polyploidal Giant Cancer Cell) among other names.⁷ In some embodiments, the TFC may be referred to as the fused cell or the giant cell.

In some embodiments, the TFC is unique in that it expresses EpCAM and CD163 markers.⁷ EpCAM expression is restricted to cells of epithelial lineage and CD163 expression is restricted to macrophage lineage. In some embodiments, there are no normal cells that express both of these antigens.

In some embodiments, the subject matter described herein relates to targeting two antigens to eliminate the TFC as part of cancer therapy in patients. In some embodiments, the antigens are a marker of epithelial cell lineage and a marker of macrophage cell lineage. In some embodiments, the antigens are EpCAM and CD163. In some embodiments, the two antigens are targeted with any of the bispecific molecules recognizing the two antigens described herein, including, but not limited to, a bispecific antibody recognizing the two antigens.

In some embodiments, to avoid targeting cells that express either of these antigens alone, the Boolean logic operator AND system can be used. In some embodiments, this system targets only the TFC due to the presence of both antigens (EpCAM and CD163). In some embodiments, this strategy spares cells that express either CD163 (macrophage lineage) or EpCAM (epithelial lineage) alone.

Boolean operators can form the basis of mathematical sets and database logic allowing the connection of search terms together to either narrow or broaden the search results. There are three basic Boolean operators—AND, OR, and NOT. Using the AND operator will narrow the search results because all search terms must be present in the resulting records. Logic-based models with only two states are known as Boolean models. In some embodiments, the principles of Boolean logic gates, that are primarily used in design and function of integrated circuits, are implemented herein to sense one or more inputs and integrate these inputs to produce the desired biological output. In some embodiments, the logic AND gate produces an output in the presence of all its designated inputs. In some embodiments of the subject matter disclosed here, the logic AND gate has been implemented in designing molecular circuits. In some embodiments, a cytotoxic response (output) can be enabled only when both the antigens (input) EpCAM and CD163 are present on the same cell.

In some embodiments, one or more of the approaches described below can be utilized when targeting the two antigens on the TFC. In some embodiments, the approaches are based on large biomolecules (e.g., proteins). In some embodiments, the approaches are based on adoptive cell therapy (e.g., CAR-T).

In some embodiments, the subject matter disclosed herein relates to a method of selectively targeting a cancer cell, the method comprising targeting at least two antigen binding regions, wherein each antigen binding region binds a different antigen on The First Cell (TFC) of a cancer. In some embodiments, the first antigen is a marker of epithelial cell lineage. In some embodiments, the marker of epithelial cell lineage is any one of the markers in FIG. 2. In some embodiments, the marker of epithelial cell lineage is epithelial cellular adhesion molecule (EpCAM). In some embodiments, the second antigen is a marker of macrophage cell lineage. In some embodiments, the marker of macrophage cell lineage is any one of the markers in FIG. 1. In some embodiments, the marker of macrophage cell lineage is CD163. In some embodiments TFC expresses one or more of the markers in FIG. 2 and one or more of the markers in FIG. 1. In some embodiments, for example in myeloid leukemias or cancers of myeloid origin, the first antigen and the second antigen are both markers of macrophage cell lineage. In some embodiments, both markers of macrophage cell lineage are selected from any one of the markers in FIG. 1. In some embodiments the first antigen is CD117, CD34, CD123, or any combination thereof and the second antigen is CD163. In some embodiments, the cancer comprises a solid tumor. In some embodiments, the cancer is breast cancer, brain tumor, gastrointestinal cancer including stomach cancer and colorectal cancer, pancreatic cancer, kidney cancer, liver cancer, lung cancer, thymic carcinoma, ovarian cancer, prostate cancer, or endometrial cancer. In some embodiments, the cancer is a liquid cancer. In some embodiments, the liquid cancer is leukemia, lymphoma, or myeloma. In some embodiments, the liquid cancer is a B-cell malignancy, including but not limited to multiple myeloma, B-cell lymphoma, diffuse large B-cell lymphoma (DLBCL), non-Hodgkin lymphomas (NHL), or chronic lymphocytic leukemia (CLL). In some embodiments, the liquid cancer is acute myeloid leukemia (AML). In some embodiments, the liquid cancer is a myeloid neoplasm. In some embodiments, the liquid cancer is myelodysplastic syndrome (MDS), myeloproliferative neoplasm (MPN), MDS/MPN overlap syndrome, acute myeloid leukemia or chronic myeloid leukemia. In some embodiments, the TFC is a metastatic TFC. In some embodiments, the bispecific molecule is a bispecific antibody or antigen binding fragment thereof. In some embodiments, the bispecific antibody or antigen binding fragment thereof is conjugated to a toxin. In some embodiments, the bispecific molecule comprises bi-nanobodies, DARPins, BiTE, tandAbs, DARTs, DART-Fc, scFv, scFv-HAS-scFV, and DNL-Fab3. In some embodiments, the bispecific molecule is a bispecific chimeric antigen receptor (CAR). In some embodiments, the bispecific molecule is a co-latching orthogonal cage-key pRotein (Co-LOCKR) comprising a first polypeptide and a second polypeptide, wherein the first polypeptide and the second polypeptide each bind a different antigen on TFC of a cancer.

In some embodiments, the bispecific molecule is a Co-LOCKR comprising a first polypeptide, a second polypeptide, and a third polypeptide, wherein the first polypeptide and the second polypeptide each bind a different antigen on TFC of a cancer, wherein the third polypeptide binds a CAR, and wherein the third polypeptide is operably linked to the first polypeptide or the second polypeptide. In some embodiments, there is a conformation change of the first and the second polypeptides after binding to their respective antigen. In some embodiments, the conformation change causes the third polypeptide to be exposed to surrounding proteins. In some embodiments, the CAR is expressed on the surface of a T cell. In some embodiments, the bispecific CAR is a synNotch CAR. In some embodiments, the bispecific molecule comprises a designed ankyrin repeat protein (DARPin). In some embodiments the first and second polypeptides of the Co-LOCKR bind their respective antigens on TFC of a cancer (e.g., EpCAM and CD163) using a DARPin domain.

### Bispecific Molecules of the Invention

In certain aspects, the invention provides a bispecific molecule comprising at least two antigen binding regions, wherein each antigen binding region binds a different antigen on The First Cell (TFC) of a cancer.

In some embodiments, the bispecific molecule is a bispecific antibody, functional equivalent thereof, antigen binding fragment thereof, a derivative thereof, or an antibody-like bispecific molecule. Such molecules are known in the art, and include, but are not limited to, molecules with full length heavy and light chains, full length heavy chains, full length light chains, Fab fragments, single chain Fv (scFv) fragments, divalent single chain antibodies or diabodies, each of which are specific to the target antigen, single domain antibodies, or one or more peptides specific to the target antigens. Various bispecific molecule formats are known in the art, for example, as described in FIG. 2 of Konterman R. E. et al., Bispecific Antibodies, Drug Discov. Today 20 (July (7)) (2015) 838-847 and FIG. 1 of Suurs F. V., et al., A review of bispecific antibodies and antibody constructs in oncology and clinical challenges, Pharmacol. Ther. 2019 September; 201:103-119, the contents of each of which is hereby incorporated by reference in their entirety. Bispecific molecules of the invention include, but are not limited to, immunoglobulin (Ig)-like bispecific antibodies as well as smaller bispecific molecules, most of which do not have an Fc region including, but not limited to, bi-nanobodies, DARPins, BiTE, tandAbs, DARTs, DART-Fc, scFv, scFv-HAS-scFV, and DNL-Fab3. There also numerous alternatives, such as affibodies, peptides, and Co-LOCKR. In fact, almost any molecule that binds a given antigen on TFC of a cancer with high affinity can be used as an antigen binding region, as will be appreciated by those of skill in the art. Methods to determine whether a bispecific molecule binds to a given antigen with high affinity are known to those of skill in the art, including but not limited to direct and indirect solid-phase assays such as ELISA and Biacore.

The structural nature of IgG antibodies is such that there are two antigen binding sites, both of which are specific for the same epitope. They are therefore monospecific. Bispecific antibodies are antibodies that have binding specificities for at least two different epitopes. Bispecific antibodies have broad applications for tumor immunotherapy because their clinical therapeutic effects can be superior to those of monoclonal antibodies. In some embodiments, the subject matter described herein relates to a bispecific antibody, functional equivalent thereof, antigen binding fragment thereof, a derivative thereof, or an antibody-like bispecific molecule, which binds to two different antigens on a cancer cell. In some embodiments, the bispecific antibody, functional equivalent thereof, antigen binding fragment thereof, a derivative thereof, or an antibody-like bispecific molecule binds to two different antigens on the surface of a TFC. In some embodiments, one antigen is an epithelial cell lineage marker and the other antigen is a macrophage cell lineage marker. In some embodiments, the epithelial cell lineage marker is EpCAM. In some embodiments, the macrophage cell lineage marker is CD163. In some embodiments, the bispecific antibody, functional equivalent thereof, antigen binding fragment thereof, a derivative thereof, or an antibody-like bispecific molecule binds to two different antigens one selected from FIG. 1 and the other selected from FIG. 2. In some embodiments, both antigens are macrophage cell lineage markers. In some embodiments the first antigen is CD117, CD34, CD123, or any combination thereof and the second antigen is CD163. In some embodiments, the bispecific antibody, functional equivalent thereof, antigen binding fragment thereof, a derivative thereof, or an antibody-like bispecific molecule binds to two different antigens selected from FIG. 1.

In some embodiments, the invention provides a bispecific molecule comprising two antigen binding regions, wherein the first antigen binding region binds EpCAM and the second antigen binding regions bind to CD163. Antigen binding regions specific for EpCAM are described herein, including but not limited to in the section titled “Antigen Binding Regions or Domains Specific for EpCAM.” Antigen binding regions specific for CD163 are described herein, including but not limited to in the section titled “Antigen Binding Regions or Domains Specific for CD163.” A bispecific molecule of the invention includes any of the antigen binding regions specific for EpCAM in combination with any of the antigen binding regions specific for CD163.

There are various platforms for generating different types of bispecific antibodies. Some technologies generating bispecific antibodies are based on the heterologous recombination of heavy chains and light chains. Different strategies to generate bispecific antibodies derived from the antigen binding site of two different antibodies are known in the art, for example, as described in Konterman R. E. et al., Bispecific Antibodies, Drug Discov. Today 20 (July (7)) (2015) 838-847, the contents of which is hereby incorporated by reference in its entirety.

In certain aspects, also provided are pharmaceutical compositions comprising the above-described bispecific molecules, polynucleotides encoding the bispecific molecules, vectors comprising a polynucleotide encoding the bispecific molecules, viruses comprising a polynucleotide encoding the bispecific molecules, genetically engineered cells, transformed or transduced host cells, comprising the bispecific molecules and/or polynucleotides encoding the bispecific molecules.

### CAR-T Therapy

Chimeric antigen receptor technology (CAR-T) therapy is a type of cancer treatment in which a patient's own T cells are programmed to attack cancer cells in the body. In this therapy T cells can be harvested from the patient's blood. Blood from a vein in the patient's arm is allowed to pass through an apheresis machine, which removes the white blood cells, including the T cells, and sends the rest of the blood back to the patient. Then, in laboratory conditions, a gene for a specific receptor that binds to a certain protein on the patient's cancer cells can be introduced into the harvested T cells. The receptor is called a chimeric antigen receptor (CAR). Large numbers of these engineered CAR-T cells can be grown in laboratory conditions and can be infused into the patient's bloodstream. The CAR-T-cell therapy has a success rate of 30% to 40% for lasting remission, with no additional treatment. In some embodiments, the methods described herein can be administered in conjunction with CAR T therapy.

In certain aspects, the bispecific molecule of the invention is a bispecific CAR comprising at least two antigen binding regions, wherein each antigen binding region binds a different antigen on The First Cell (TFC) of a cancer. In some embodiments, the bispecific CAR binds to two different antigens on the surface of a TFC. In some embodiments, one antigen is an epithelial cell lineage marker and the other antigen is a macrophage cell lineage marker. In some embodiments, the epithelial cell lineage marker is EpCAM. In some embodiments, the macrophage cell lineage marker is CD163. In some embodiments, the bispecific CAR binds to two different antigens one selected from FIG. 1 and the other selected from FIG. 2. In some embodiments, both antigens are macrophage cell lineage markers. In some embodiments the first antigen is CD117, CD34, CD123, or any combination thereof and the second antigen is CD163. In some embodiments, both antigens are macrophage cell lineage markers. In some embodiments, the bispecific CAR binds to two different antigens selected from FIG. 1.

In some embodiments, the invention provides a bispecific CAR comprising two antigen binding regions, wherein the first antigen binding region binds to EpCAM and the second antigen binding region binds to CD163. Antigen binding regions specific for EpCAM are described herein, including but not limited to in the section titled “Antigen Binding Regions or Domains Specific for EpCAM.” Antigen binding regions specific for CD163 are described herein, including but not limited to in the section titled “Antigen Binding Regions or Domains Specific for CD163.” A bispecific CAR of the invention includes any of the antigen binding regions specific for EpCAM in combination with any of the antigen binding regions specific for CD163.

In certain aspects, also provided are pharmaceutical compositions comprising the above-described bispecific CAR, polynucleotides encoding the bispecific CAR, vectors comprising a polynucleotide encoding the bispecific CAR, viruses comprising a polynucleotide encoding the bispecific CAR, and genetically engineered cells comprising the bispecific CAR and/or polynucleotides encoding the bispecific CAR.

In some embodiments, approaches to avoid targeting cells that express only one antigen can be used. Such approaches are known in the art and include, but are not limited to, Co-LOCKR, SynNotch CAR cells, and combinatorial antigen recognition systems.

### Co-LOCKR Cell Targeting

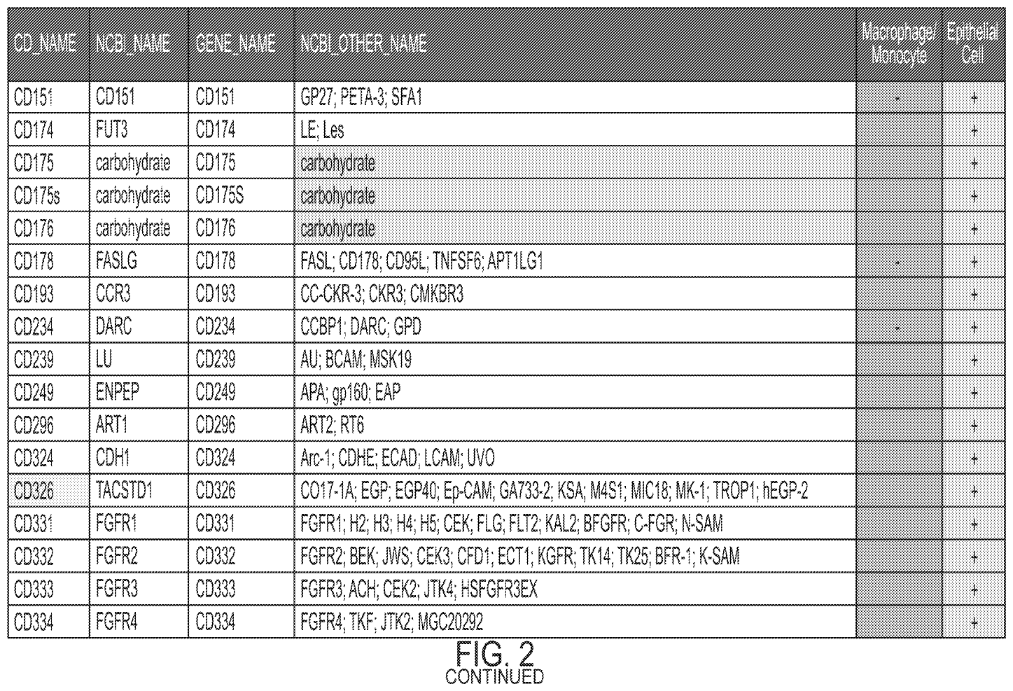

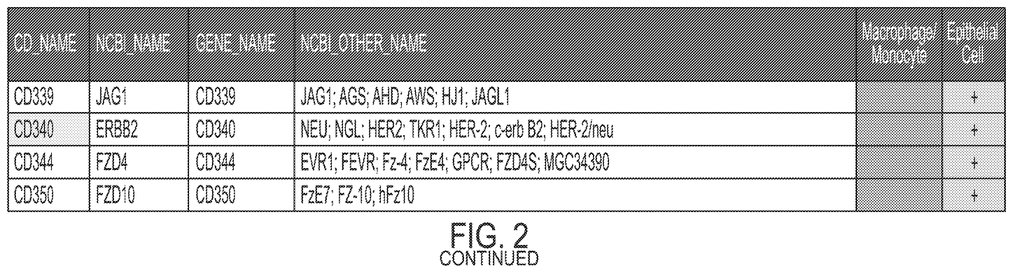

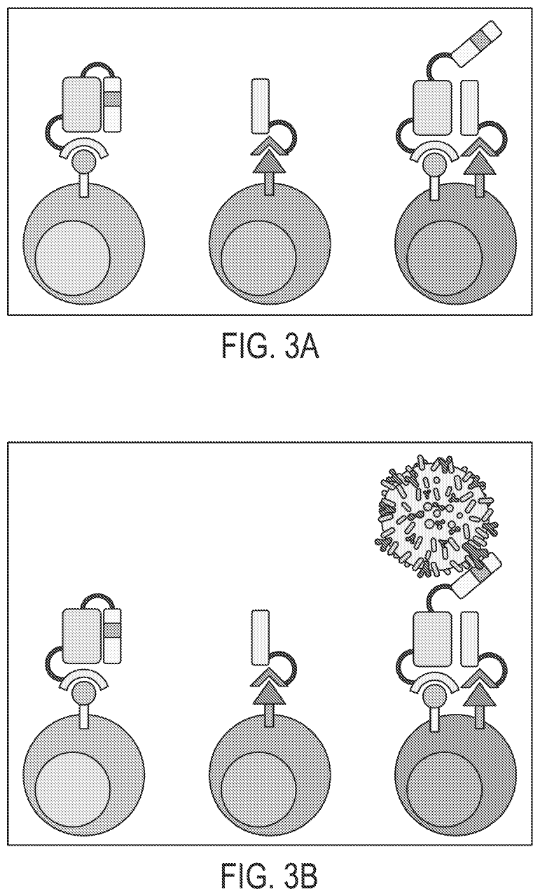

Co-LOCKR are colocalization-dependent protein switches that perform AND, OR, and NOT logic operations.¹⁵ The system includes designed nanoscale devices made of synthetic proteins that target a therapeutic agent or antibody only to cells with specific, predetermined combinations of cell surface markers. In some embodiments, these protein switches perform AND logic on the cell surface. In some embodiments, Co-LOCKR proteins perform 2- and 3-input logic operations in mixed cell populations. In some embodiments, the Latching Orthogonal Cage-Key pRotein (LOCKR) switch consists of a structural “Cage” protein that uses a “Latch” domain to sequester a functional peptide in an inactive conformation until binding of a separate “Key” protein induces a conformational change that permits binding to an “Effector” protein. In some embodiments, Cage, Key, and Effector bind in a three-way equilibrium, and the sensitivity of the switch can be tuned by adjusting the relative Cage-Latch and Cage-Key affinities. Additional embodiments of Co-LOCKR systems are described in Lajoie, M. J., et al., Designed protein logic to target cells with precise combinations of surface antigens, Science. 2020 Sep. 25; 369(6511): 1637-1643, which is incorporated herein in its entirety. In some embodiments, the synthetic proteins are molecular switches that, when separated, have no effect. But when they are combined on the surface of a targeted cell, they change conformation, activating a molecular beacon. These beacons on a cell surface can guide a predetermined biological activity, for example cell eliminating, to a specific, targeted cell. In some embodiments, these molecular beacons recruit a CAR-T cell, which specifically binds the molecular beacon as shown in FIGS. 3A-B. In some embodiments, the molecular beacons comprise the Bim-Bcl2 system. In some embodiments, Bim is encoded into the Latch of the LOCKR system as a sequestered peptide. In some embodiments, Bcl2 is used as the Effector of the LOCKR system.

In some embodiments, the Co-LOCKR system comprises a CAR-T cells that binds to Bcl2 which is used as the effector molecule of the system. In one embodiment, the CAR-T system described herein comprises a Bcl2 CAR sequence comprising the amino acid sequence:

(SEQ ID NO: 1)
METDTLLLWVLLLWVPGSTGDYKDEYPYDVPDYAGSAHAGRTGYDNREI
VMKYIHYKLSQRGYEWDAGDDAEENRTEAPEGTESEVVHRALRDAGDDF
ERRYRRDFAEMSSQLHLTPDTARQRFETVVEELFRDGVNWGRIVAFFEF
GGVMCVESVNREMSPLVDNIAEWMTEYLNRHLHTWIQDNGGWDAFVELY
GPSMRGGGGSGGGGSESKYGPPCPPCPAPPVAGPSVFLFPPKPKDTLMI
SRTPEVTCVVVDVSQEDPEVQFNWYVDGVEVHNAKTKPREEQFQSTYRV
VSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGKMF
WVLVVVGGVLACYSLLVTVAFIIFWVRSKRSRGGHSDYMNMTPRRPGPT
RKHYQPYAPPRDFAAYRSRVKFSRSADAPAYQQGQNQLYNELNLGRREE
YDVLDKRRGRDPEMGGKPRRKNPQEGLYNELQKDKMAEAYSEIGMKGER
RRGKGHDGLYQGLSTATKDTYDALHMQALPPRLEGGGEGRGSLLTCGDV
EENPGPRMLLLVTSLLLCELPHPAFLLIPRKVCNGIGIGEFKDSLSINA
TNIKHFKNCTSISGDLHILPVAFRGDSFTHTPPLDPQELDILKTVKEIT
GFLLIQAWPENRTDLHAFENLEIIRGRTKQHGQFSLAVVSLNITSLGLR
SLKEISDGDVIISGNKNLCYANTINWKKLFGTSGQKTKIISNRGENSCK
ATGQVCHALCSPEGCWGPEPRDCVSCRNVSRGRECVDKCNLLEGEPREF
VENSECIQCHPECLPQAMNITCTGRGPDNCIQCAHYIDGPHCVKTCPAG
VMGENNTLVWKYADAGHVCHLCHPNCTYGCTGPGLEGCPTNGPKIPSIA
TGMVGALLLLLVVALGIGLFM.

In some embodiments, the Bcl2 Effector of the LOCKR system is a Bcl CAR having at least 60%, 65%, 70%, 75%, 80%, 85%, 90%, 95%, or 99% identity with SEQ ID NO: 1. In some embodiments, the Bcl2 Effector of the LOCKR system is a Bcl CAR comprising the sequence of SEQ ID NO: 1. In some embodiments, the Bcl2 Effector of the LOCKR system is a Bcl CAR consisting of the sequence of SEQ ID NO: 1. In some embodiments, the Bcl CAR is part of a CAR-T system.

In some embodiments, the bispecific molecule of the invention is a Co-LOCKR comprising a first polypeptide and a second polypeptide, wherein the first polypeptide and the second polypeptide bind a different antigen on The First Cell (TFC) of a cancer. In some embodiments, the bispecific molecule is a Co-LOCKR comprising a first polypeptide, a second polypeptide, and a third polypeptide, wherein the first polypeptide and the second polypeptide each bind a different antigen on TFC of a cancer, wherein the third polypeptide binds a CAR, and wherein the third polypeptide is operably linked to the first polypeptide or the second polypeptide. In some embodiments, one antigen is an epithelial cell lineage marker and the other antigen is a macrophage cell lineage marker. In some embodiments the subject matter described herein relates to generating a bispecific Co-LOCKR system that recognize EpCAM and CD163. In some embodiments, the epithelial cell lineage marker bound by the first polypeptide is EpCAM. In some embodiments, the macrophage cell lineage marker bound by the second polypeptide is CD163. In some embodiments, the first polypeptide binds to any antigen selected from FIG. 1 and the second polypeptide binds to any antigen selected from FIG. 2. In some embodiments, both antigens are macrophage cell lineage markers. In some embodiments the first antigen is CD117, CD34, CD123, or any combination thereof and the second antigen is CD163. In some embodiments, the first polypeptide binds to any antigen selected from FIG. 1 and the second polypeptide binds to any other antigen selected from FIG. 1. In some embodiments, upon binding of the first and second polypeptides to a TFC of a cancer, a molecular beacon is activated, for example to guide a killer T-cell to kill the TFC of a cancer.

In some embodiments, the invention provides a bispecific Co-LOCKR comprising a first polypeptide and a second polypeptide, wherein the first polypeptide comprises an antigen binding region that binds EpCAM and the second polypeptide comprises an antigen binding region that binds to CD163. Antigen binding regions specific for EpCAM are described herein, including but not limited to in the section titled “Antigen Binding Regions or Domains Specific for EpCAM.” Antigen binding regions specific for CD163 are described herein, including but not limited to in the section titled “Antigen Binding Regions or Domains Specific for CD163.” A bispecific Co-LOCKR of the invention includes use of any of the antigen binding regions specific for EpCAM in combination with any of the antigen binding regions specific for CD163.

In some embodiments, the invention provides a bispecific Co-LOCKR comprising a first polypeptide and a second polypeptide, wherein the first polypeptide comprises key or cage domain and an antigen binding region that binds EpCAM and the second polypeptide comprises a key or cage domain and an antigen binding region that binds to CD163, wherein if the first polypeptide comprises a key domain the second polypeptide comprises a cage domain or wherein if the first polypeptide comprises a cage domain the second polypeptide comprises a key domain. Sequences encoding bispecific Co-LOCKRs can further comprises a signal peptide, a purification tag, and/or a protease site. A person of skill in the understands that a signal peptide and/or a purification tag are cleaved or removed from any polypeptide before administration of a bispecific Co-LOCKR to a subject in need thereof. Exemplary key and cage domains as well as signal peptides, purification tags, and protease sites are described in Example 2.

In certain aspects, also provided are pharmaceutical compositions comprising the Co-LOCKR described herein, polynucleotides encoding the Co-LOCKR described herein, viruses comprising a polynucleotide encoding the Co-LOCKR described herein, and genetically engineered cells comprising the Co-LOCKR described herein and/or polynucleotides encoding the Co-LOCKR described herein.

### Split-CAR-T

A split chimeric antigen receptor T cell (split-CAR-T) system includes two modules—one with a CAR and other with chimeric costimulatory receptor (CCR). Binding of both CAR and CCR is required for full T-cell activation. The CAR and the CCR can be expressed in the same T-cell to achieve balanced signaling and maximal T-cell cytotoxic activity on a target cell expressing two different targeted antigens such as The First Cell. In some embodiments, the CAR module comprises a polypeptide comprising an antigen binding domain specifically recognising a first antigen and CD3z signaling domain. In some embodiments, the CAR module includes: 1) a signal peptide for membrane targeting (derived from GM-CSF or CD8 alpha), 2) ScFv sequences (derived from either anti-EpCAM or anti-CD163 antibodies), 3) a hinge region (derived from CD8), 4) a transmembrane domain (derived from CD8), and 5) a CD3z signaling domain.

In some embodiments, the CCR module comprises a polypeptide comprising an antigen binding domain specifically recognising a second antigen and two or more co-stimulatory domains. In some embodiments, the CCR module includes: 1) a signal peptide for membrane targeting (derived from GM-CSF), 2) ScFv sequences (derived from either anti-EpCAM or anti-CD163 antibodies), 3) a hinge region (derived from CD28), 4) a transmembrane domain (derived from CD28), and 5) a CD28 co-stimulatory domain, and 6) a 4-1BB co-stimulatory domain.

In some embodiments, the invention provides a bispecific split-CAR-T comprising a first polypeptide and a second polypeptide, wherein the first polypeptide comprises an antigen binding region that binds EpCAM and the second polypeptide comprises an antigen binding region that binds to CD163. Antigen binding regions specific for EpCAM are described herein, including but not limited to in the section titled “Antigen Binding Regions or Domains Specific for EpCAM.” Antigen binding regions specific for CD163 are described herein, including but not limited to in the section titled “Antigen Binding Regions or Domains Specific for CD163.” A bispecific split-CAR-T of the invention includes use of any of the antigen binding regions specific for EpCAM in combination with any of the antigen binding regions specific for CD163.

In some embodiments, the invention provides a bispecific split-CAR-T comprising a first polypeptide and a second polypeptide, wherein the first polypeptide comprises an antigen binding region that binds EpCAM and a spacer domain, a transmembrane domain, and a CD3z signaling domain and the second polypeptide comprises an antigen binding region that binds to CD163, a spacer domain, a transmembrane domain, a first co-stimulatory domain, and a second co-stimulatory domain. In some embodiments, the invention provides a bispecific split-CAR-T comprising a first polypeptide and a second polypeptide, wherein the first polypeptide comprises an antigen binding region that binds CD163 and a spacer domain, a transmembrane domain, and a CD3z signaling domain and the second polypeptide comprises an antigen binding region that binds to EpCAM, a spacer domain, a transmembrane domain, a first co-stimulatory domain, and a second co-stimulatory domain. The polypeptide sequences can further comprise a signal peptide. In some embodiments the spacer domain is a CD8 hinge domain. In some embodiments the spacer domain is a CD28 hinge domain. In some embodiments the transmembrane domain is a CD8 transmembrane domain. In some embodiments the transmembrane domain is a CD28 transmembrane domain. In some embodiments the first co-stimulatory domain is a CD28 co-stimulatory domain. In some embodiments the second co-stimulatory domain is a 4-1BB co-stimulatory domain. A person of skill in the understands that a signal peptide is cleaved or removed from any polypeptide during post-translational processing. Exemplary spacer domains, transmembrane domains, and co-stimulatory domains as well as signal peptides are described in Example 3.

In some embodiments, the invention provides a bispecific split-CAR-T comprising a first polypeptide and a second polypeptide, wherein the first polypeptide comprises an antigen binding region that binds to EpCAM and a CD8 hinge domain, a CD8 transmembrane domain, and a CD3z signalling domain and the second polypeptide comprises an antigen binding regions bind to CA163, a CD28 hinge domain, a CD28 transmembrane domain, a CD28 co-stimulatory domain, and a 4-1BB co-stimulatory domain. In some embodiments, the invention provides a bispecific split-CAR-T comprising a first polypeptide and a second polypeptide, wherein the first polypeptide comprises an antigen binding region that binds to CD163 and a CD8 hinge domain, a CD8 transmembrane domain, and a CD3z signaling domain and the second polypeptide comprises an antigen binding region that binds to EpCAM, a CD28 hinge domain, a CD28 transmembrane domain, a CD28 co-stimulatory domain, and a 4-1BB co-stimulatory domain.

In certain aspects, also provided are pharmaceutical compositions comprising the split-CAR-T described herein, polynucleotides encoding the split-CAR-T described herein, viruses comprising a polynucleotide encoding the split-CAR-T described herein, and genetically engineered cells comprising the split-CAR-T described herein and/or polynucleotides encoding the split-CAR-T described herein.

### SynNotch CAR Cells^{16,17}

Synthetic Notch (synNotch) pathways can drive pre-determined functional responses in mammalian cells. Individual synNotch pathways do not share common signaling intermediates, which makes the pathways functionally orthogonal. Thus, multiple synNotch receptors can be used in the same cell to achieve integration of multiple external cues, including Boolean response programs. SynNotch-CAR T cells are prime-and-kill molecular circuits. The synNotch receptors prime and activate the CAR T cells only when all of the relevant antigens are present on the target cell. This allows the CAR T cells only target cancerous cells and to spare the normal cells. Additional, CAR-based approaches include SUPRA CAR,^{19,20} RevCAR,^{21,22} and AvidCAR.²³ In some embodiments, the bispecific CAR is a SynNotch CAR. In some embodiments, the TFC is targeted by the bispecific molecule using a Co-LOCKR system of protein switches. In some embodiments, the TFC is targeted by the bispecific molecule using a synCAR system, SUPRA CAR system, RevCAR system, or AvidCAR system. In some embodiments, the TFC is targeted by a bispecific antibody using combinatorial antigen recognition system. In some embodiments, the TFC is targeted by a bispecific antibody conjugated to a toxin.

### Combinatorial Antigen Recognition System¹⁸

A combinatorial antigen recognition system can promote selective tumor eradication by engineered T cells. It allows the engineered T cells to be specific for a tumor in the absence of a truly tumor-specific target antigen.

### DARPins

Designed ankyrin repeat proteins (DARPins) are genetically engineered antibody mimetic proteins. DARPins bind their target proteins with high specificity and high-affinity. They can be derived from ankyrin repeat proteins, a class of binding proteins responsible for diverse cellular functions. In some embodiments, the DARPins consist of two or more repeat polypeptide motifs and have a hydrophobic core protected by the N- and C-terminal caps.

In some embodiments, the bispecific molecule of the invention is a DARPin molecule comprising at least a first polypeptide and a second polypeptide, wherein the first polypeptide and the second polypeptide bind a different antigen on The First Cell (TFC) of a cancer. In some embodiments, one antigen is an epithelial cell lineage marker and the other antigen is a macrophage cell lineage marker. In some embodiments the subject matter described herein relates to generating a bispecific DARPin molecule that recognizes EpCAM and CD163. In some embodiments, the epithelial cell lineage marker bound by the first polypeptide is EpCAM. In some embodiments, the macrophage cell lineage marker bound by the second polypeptide is CD163. In some embodiments, the first polypeptide binds to any antigen selected from FIG. 1 and the second polypeptide binds to any antigen selected from FIG. 2. In some embodiments, both antigens are macrophage cell lineage markers. In some embodiments the first antigen is CD117, CD34, CD123, or any combination thereof and the second antigen is CD163. In some embodiments, the first polypeptide binds to any antigen selected from FIG. 1 and the second polypeptide binds to any other antigen selected from FIG. 1. In some embodiments, the bispecific DARPin molecule guides a killer T-cell to kill the TFC of a cancer. In some embodiments, the CAR antigen recognition domain comprises a bispecific DARPin molecule. In some embodiments, the antigen binding domains of the Co-LOCKR system comprise a DARPin. In some embodiments, one or more DARPins which specifically bind EpCAM and CD163 on a target cell are identified by screening one or more libraries of DAPRins. In some embodiments, the one of more libraries of DAPRins are commercially available.

### Single-Chain Variable Fragment

A single-chain variable fragment (scFv) is a fusion protein of the variable regions of the heavy (VH) and light chains (VL) of one or more immunoglobulins. The two chains can be connected with a short linker peptide of 10 to about 25 amino acids. The linker can be rich in glycine for flexibility, as well as serine or threonine for solubility. The linker can either connect the N-terminus of the VH with the C-terminus of the VL, or vice versa. This protein retains the specificity of the original one or more immunoglobulins. scFV can be expressed as the antigen binding domains of any of the bispecific molecules described herein. scFv can be expressed as antigen-binding domains of CARs. In some embodiments, multi-valent scFv can be engineered by linking two or more scFv fragments together. In some embodiments, multi-valent scFv can be engineered into bispecific tandem scFvs, known as bi-specific T-cell engagers (BiTE antibody constructs).

In some embodiments, the bispecific molecule of the invention comprises at least two scFvs, wherein the antigen binding sites of each scFv each bind a different antigen on The First Cell (TFC) of a cancer. In some embodiments, one antigen is an epithelial cell lineage marker and the other antigen is a macrophage cell lineage marker. In some embodiments the subject matter described herein relates to generating a bispecific molecule comprising at least two scFvs that recognizes EpCAM and CD163. In some embodiments, the epithelial cell lineage marker bound by the first scFV is EpCAM. In some embodiments, the macrophage cell lineage marker bound by the second scFV is CD163. In some embodiments, the first scFV binds to any antigen selected from FIG. 1 and the second scFV binds to any antigen selected from FIG. 2. In some embodiments, both antigens are macrophage cell lineage markers. In some embodiments the first antigen is CD117, CD34, CD123, or any combination thereof and the second antigen is CD163. In some embodiments, the first scFV binds to any antigen selected from FIG. 1 and the second scFV binds to any other antigen selected from FIG. 1. In some embodiments, the bispecific scFv fragment guides a killer T-cell to kill the TFC of a cancer. In some embodiments, the CAR antigen recognition domain comprises a bispecific scFv fragment. Embodiments and sequences of antibody molecules that specifically bind EpCAM are disclosed in U.S. Pat. No. 7,632,925 B2, the content of which is hereby incorporated by reference in its entirety. Embodiments and sequences of antibody molecules that specifically bind CD163 are disclosed in United States Patent Application Publication No.: US 2017/0119790 A1, U.S. Pat. Nos. 9,724,426, and 11,034,770 the content of each which are hereby incorporated by reference in their entireties.

### Drug-Conjugated Bispecific Antibodies

In some embodiments, the subject matter described herein relates to generating a drug-conjugated bispecific antibodies. In some embodiments, the drug is a toxin. In some embodiments, the toxin is a chemotherapy agent. In some embodiments, the chemotherapy agent is emtansine. In some embodiments, the drug is methotrexate, thioguanine, 5-fluorouracil, cytosine arabinoside (ara-C), cisplatin, actinomycin D, anthracyclines, or Vinca alkaloids. In some embodiments, the drug is a microtubule-disrupting agent. In some embodiments, the microtubule-disrupting agent is auristatin. In some embodiments, the microtubule-disrupting agent comprises maytansinoids. In some embodiments, the drug is a DNA-damaging agent. In some embodiments, the DNA-damaging agent is calicheamicin. In some embodiments, the DNA-damaging agent is duocarmycin. In some embodiments, the DNA-damaging agent is doxorubicin. In some embodiments, the toxin kills the target cancer cell. In some embodiments, the drug is conjugated to the bispecific antibody via a biotin-streptavidin interaction. In some embodiments, the drug is covalently conjugated to the bispecific antibody. In some embodiments, the drug is conjugated to the antibody via a linker. Additional embodiments of antibody-drug conjugates are disclosed in Khongorzul, P., Antibody—Drug Conjugates: A Comprehensive Review, Mol Cancer Res., 2020, 18(1):3-19, which is incorporated herein in its entirety.

In some embodiments, the goal of drug-conjugated antibody therapy is to deliver a highly toxic drug to its target cell using a specific carrier. In some embodiments, the administration of such therapy is done intravenously into the bloodstream to avoid gastric acid and proteolytic enzyme degradation of the antibody. In some embodiments, upon recognition of its target cell, the drug conjugated antibody and its antigen are internalized into the cell via receptor-mediated endocytosis. In some embodiments, internalization results in release of the free cytotoxic drug into the cytoplasm, where the drug interferes with the cellular mechanisms, induces apoptosis, and/or ultimately cell death.

In certain aspects, the bispecific molecule of the invention is a drug-conjugated bispecific antibody comprising at least two antigen binding regions, wherein each antigen binding region binds a different antigen on The First Cell (TFC) of a cancer. In some embodiments, the bispecific antibody binds to two different antigens on the surface of a TFC. In some embodiments, one antigen is an epithelial cell lineage marker and the other antigen is a macrophage cell lineage marker. In some embodiments, the epithelial cell lineage marker is EpCAM. In some embodiments, the macrophage cell lineage marker is CD163. In some embodiments, the bispecific antibody binds to two different antigens one selected from FIG. 1 and the other selected from FIG. 2. In some embodiments, both antigens are macrophage cell lineage markers. In some embodiments the first antigen is CD117, CD34, CD123, or any combination thereof and the second antigen is CD163. In some embodiments, the bispecific antibody binds to two different antigens one selected from FIG. 1.

In some embodiments, the invention provides a drug-conjugated bispecific antibody comprising two antigen binding regions, wherein the first antigen binding region binds to EpCAM and the second antigen binding region binds to CD163. Antigen binding regions specific for EpCAM are described herein, including but not limited to in the section titled “Antigen Binding Regions or Domains Specific for EpCAM.” Antigen binding regions specific for CD163 are described herein, including but not limited to in the section titled “Antigen Binding Regions or Domains Specific for CD163.” A drug-conjugated bispecific antibody of the invention includes any of the antigen binding regions specific for EpCAM in combination with any of the antigen binding regions specific for CD163.

In certain aspects, also provided are pharmaceutical compositions comprising the above-described drug-conjugated bispecific antibodies, polynucleotides encoding the drug-conjugated bispecific antibodies, vectors comprising a polynucleotide encoding the drug-conjugated bispecific antibodies, viruses comprising a polynucleotide encoding the drug-conjugated bispecific antibodies, and genetically engineered cells comprising the bispecific antibodies and/or polynucleotides encoding the drug-conjugated bispecific antibodies.

### Engineered Hybrid Cells

In some embodiments, the subject matter described herein relates to engineering of a cell or a population of cells expressing at least one marker of epithelial cell lineage and at least one marker of macrophage cell lineage. In some embodiments, the at least one marker of epithelial cell lineage is any one of the markers in FIG. 2. In some embodiments, the at least one marker of epithelial cell lineage is epithelial cellular adhesion molecule (EpCAM). In some embodiments, the at least one marker of macrophage cell lineage is any one of the markers in FIG. 1. In some embodiments, the at least one marker of macrophage cell lineage is CD163. In some embodiments, the cells express either EpCAM or CD163 alone or both EpCAM and CD163 markers. In some embodiments, the subject matter described herein relates to engineering of a cell or a population of cells expressing at least two markers macrophage cell lineage. In some embodiments, the at least two markers are any of CD117, CD34, CD123, or any combination thereof and CD163. In some embodiments, the subject matter described herein relates to demonstrating the specific targeting of engineered cells expressing both antigens, sparing cells that express either antigen alone. In some embodiments, the subject matter described herein relates to performing in vitro and in vivo assay studies to demonstrate the specificity and toxicity of the bi-specific molecule. In some embodiments, the target cells used in the assay are engineered to express at least one marker of epithelial cell lineage and at least one marker of macrophage cell lineage. In some embodiments, the target cells used in the assay are engineered to express at least two markers of macrophage cell lineage. In some embodiments, the target cells used in the assay comprise a population of engineered cells expressing two different antigens one selected from FIG. 1 and the other selected from FIG. 2. In some embodiments, the target cells used in the assay comprise a population of engineered cells expressing two different antigens selected from FIG. 1. In some embodiments, the target cells used in the assay comprise a population of engineered cells expressing both EpCAM and CD163 markers. In some embodiments, the subject matter described herein relates to the identification of several other antigens that show restricted lineage specific expression and can be potentially targeted when a combination of antigens from FIG. 1 and FIG. 2 are used. In some embodiments, the cancer treatment approach described herein will target and eliminate TFCs universally in all cancers.

### Antigen Binding Regions or Domains

An antibody is a heteromultimeric glycoprotein comprising at least two heavy chains and two light chains. Apart from IgM, intact antibodies are usually heterotetrameric glycoproteins composed of two identical light (L) chains and two identical heavy (H) chains. Typically, each light chain is linked to the heavy chain by disulfide bonding. Each heavy and light chain also has intrachain disulfide bridges. Each heavy chain has at one end a variable domain (VH) followed by a number of constant regions. Each light chain has a variable domain (VL) and a constant region at the other end. The light chains of antibodies from most vertebrate species can be assigned to one of two types, called kappa and lambda, based on the amino acid sequence of the constant region. The variable domain of an antibody confers binding specificity on the antibody, and certain regions exhibit a unique variability called the complementarity determining region (CDR). The more conserved part of the variable region is called the framework region (FR). The intact heavy and light chain variable domains of an antibody each contain four FRs joined by three CDRs. The CDRs in each chain are held close together by the FR region together with the CDRs from the other chain and contribute to the formation of the antigen binding site of the antibody. When non-human antibodies are prepared with respect to a particular antigen, the variable regions can be humanized by grafting CDRs derived from the non-human antibody on the FRs present in the human antibody to be modified. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, or six) which are altered with respect to the original antibody, which are also referred to as one or more CDRs derived from one or more CDRs from the original antibody.

With respect to antigen binding regions or domains that bind to a desired target derived from antibodies that bind to the desired target, such as but not limited to EpCAM and CD163, exemplary amino acid sequences of the variable light (VL) chain and variable heavy (VH) chain of these antibodies are shown below. Those of skill in the art will therefore be able to construct bispecific molecules having CDRs of said VH and VL chains, as well as antibodies and derivatives thereof, including humanized derivatives thereof, capable of binding to the epitopes recognized by these antibodies.

Affinity refers to the equilibrium constant for the reversible binding of two agents and is expressed as KD. In some embodiments, the bispecific molecules and/or antigen binding regions or domains thereof exhibit binding affinity as measured by KD (equilibrium dissociation constant) for CD163 or EpCAM in the nanomolar range (10⁻⁷ to 10⁻⁹M) or less. In certain embodiments, antibodies as described herein specifically bind to human CD163 polypeptide with a KD of less than or equal to 10 nM. In certain embodiments, antibodies as described herein specifically bind to human EpCAM polypeptide with a KD of less than or equal to 10 nM.

### Antigen Binding Regions or Domains Specific for EpCAM

Provided below is the amino acid sequence corresponding to the EpCAM (Uniprot ID: P16422):

(SEQ ID NO: 2)
MAPPQVLAFGLLLAAATATFAAAQEECVCENYKLAVNCFVNNNRQCQCT
SVGAQNTVICSKLAAKCLVMKAEMNGSKLGRRAKPEGALQNNDGLYDPD
CDESGLFKAKQCNGTSMCWCVNTAGVRRTDKDTEITCSERVRTYWIIIE
LKHKAREKPYDSKSLRTALQKEITTRYQLDPKFITSILYENNVITIDLV
QNSSQKTQNDVDIADVAYYFEKDVKGESLFHSKKMDLTVNGEQLDLDPG
QTLIYYVDEKAPEFSMQGLKAGVIAVIVVVVIAVVAGIVVLVISRKKRM
AKYEKAEIKEMGEMHRELNA.

In some embodiments, one of the antigen binding regions of the bispecific molecule binds to EpCAM. In some embodiments, one of the antigen binding regions of the bispecific molecule binds to an epitope of SEQ ID NO:2. In some embodiments the invention relates to IgG-based structures, for example a polynucleotide and polypeptide sequence encoding a heavy chain and a light chain sequence of an EpCAM-specific antibody. Embodiments and sequences of antibody molecules that specifically bind EpCAM are known in the art, including those disclosed in U.S. Pat. No. 7,632,925 B2, the content of which is hereby incorporated by reference in its entirety.

In some embodiments the antigen binding region specific to EpCAM comprises SEQ ID NO: 3, which is an EpCAM-specific DARPin:

(SEQ ID NO: 3)
DLGKKLLEAARAGQDDEVRILVANGADVNAYFGTTPLHLAAAHGRLEIV
EVLLKNGADVNAQDVWGITPLHLAAYNGHLEIVEVLLKYGADVNAHDTR
GWTPLHLAAINGHLEIVEVLLKNVADVNAQDRSGKTPFDLAIDNGNEDI
AEVLQKAAKLN.

In some embodiments, the antigen binding region specific to EpCAM comprises an amino acid sequence having at least 60%, 65%, 70%, 75%, 80%, 85%, 90%, 91%, 92%, 93%, 94%, 95%, 96%, 97%, 98%, or 99% identity with SEQ ID NO: 3. Additional details on DARPin to EpCAM can be found in Stefan, N. et al., DARPins recognizing the tumor-associated antigen EpCAM selected by phage and ribosome display and engineered for multivalency. J Mol Biol, 2011, 413(4):826-843, which is incorporated herein in its entirety.

In some embodiments the antigen binding region specific to EpCAM comprises SEQ ID NO: 4, which is a co-LOCKR Cage targeted to EpCAM by DARPin:

(SEQ ID NO: 4)
MGSHHHHHHGSGSENLYFQGSGGSDLGKKLLEAARAGQDDEVRILVANG
ADVNAYFGTTPLHLAAAHGRLEIVEVLLKNGADVNAQDVWGITPLHLAA
YNGHLEIVEVLLKYGADVNAHDTRGWTPLHLAAINGHLEIVEVLLKNVA
DVNAQDRSGKTPFDLAIDNGNEDIAEVLQKAAKLNSGSGSGKPGQASGS
ELARKLLEASTKLQRLNIRLAEALLEAIARLQELNLELVYLAVELTDPK
RIRDEIKEVKDKSKEIIRRAEKEIDDAAKESEKILEEAREAISGSGSEL
AKLLLKAIAETQDLNLRAAKAFLEAAAKLQELNIRAVELLVKLTDPATI
REALEHAKRRSKEIIDEAERAIRAAKRESERIIEEARRLIEKGSGSGSE
LARELLRAHAQLQRLNLELLRELLRALAQLQELNLDLLRLASELTDEIW
IAQELRRIGDEFNAYYADAERLIREAAAASEKISREAERLIR

In some embodiments the antigen binding region specific to EpCAM comprises an amino acid sequence having at least 60%, 65%, 70%, 75%, 80%, 85%, 90%, 91%, 92%, 93%, 94%, 95%, 96%, 97%, 98%, or 99% identity with SEQ ID NO: 4.

In some embodiments the antigen binding region specific to EpCAM comprises SEQ ID NO: 5, which is a co-LOCKR Key targeted to EpCAM by DARPin: MGSHHHHHHGSGSENLYFQGSGGSDEARKAIARVKRESKRIVEDAERLIREAAAASE KISREAERLIRGGGSGSGSGSGKPGQASGSDLGKKLLEAARAGQDDEVRILVANGAD VNAYFGTTPLHLAAAHGRLEIVEVLLKNGADVNAQDVWGITPLHLAAYNGHLEIVE VLLKYGADVNAHDTRGWTPLHLAAINGHLEIVEVLLKNVADVNAQDRSGKTPFDL AIDNGNEDIAEVLQKAAKLN (SEQ ID NO: 5)

In some embodiments the antigen binding region specific to EpCAM comprises an amino acid sequence having at least 60%, 65%, 70%, 75%, 80%, 85%, 90%, 91%, 92%, 93%, 94%, 95%, 96%, 97%, 98%, or 99% identity with SEQ ID NO: 5.

In some embodiments the antigen binding region specific to EpCAM comprises the VH chain of SEQ ID NO: 27:

(SEQ ID NO: 27)
EVQLLESGGGVVQPGRSLRLSCAASGFTFSSYGMHWVRQAPGKGLEWVA
VISYDGSNKYYADSVKGRFTISRDNSKNTLYLQMNSLRAEDTAVYYCAK
DMGWGSGWRPYYYYGMDVWGQGTTVTVSSAPTKAPDVFPL.

The CDRs are shown in bold and underlined and comprise amino acids 31 to 35 (CDRH1), 50 to 66 (CDRH2), and 98 to 117 (CDRH3). A nucleic acid encoding SEQ ID NO: 27 is SEQ ID NO: 143 of U.S. Pat. No. 7,632,925, incorporated herein by reference. CDRH1 comprises nt 91 to nt 105, CDRH2 nt 148 to nt 198, CDRH3 nt 292 to nt 351.

In some embodiments, the antigen binding region specific to EpCAM comprises an amino acid sequence having at least 60%, 65%, 70%, 75%, 80%, 85%, 90%, 91%, 92%, 93%, 94%, 95%, 96%, 97%, 98%, or 99% identity with SEQ ID NO: 27.

In some embodiments, the antigen binding region specific to EpCAM comprises a VH region comprising one or more CDR sequences selected from:

(SEQ ID NO: 33)
SYGMH
(SEQ ID NO: 34)
VISYDGSNKYYADSVKG
(SEQ ID NO: 35)
KDMGWGSGWRPYYYYGMDVW.

In some embodiments the antigen binding region specific to EpCAM comprises the VL chain of SEQ ID NO: 28:

(SEQ ID NO: 28)
ELQMTQSPSSLSASVGDRVTITCRASQSISSYLNWYQQKPGQPPKLLIY
WASTRESGVPDRFSGSESGTNYTLTISSLQPEDFATYFCQQSDSLPITF
GQGTRLDIQ.

The CDRs are shown in bold and underlined and comprise amino acids 24-34 (CDR1), 50 to 56 (CDR2), and 89 to 98 (CDR3). A nucleic acid encoding SEQ ID NO: 28 is SEQ ID NO: 147 of U.S. Pat. No. 7,632,925, incorporated herein by reference. CDRL1 comprises nt 70 to nt 102, CDRL2 nt 148 to nt 168, CDRL3 nt 265 to nt 294.

In some embodiments, the antigen binding region specific to EpCAM comprises an amino acid sequence having at least 60%, 65%, 70%, 75%, 80%, 85%, 90%, 91%, 92%, 93%, 94%, 95%, 96%, 97%, 98%, or 99% identity with SEQ ID NO: 28.

In some embodiments, the antigen binding region specific to EpCAM comprises a VL region comprising one or more CDR sequences selected from:

(SEQ ID NO: 36)
RASQSISSYLN
(SEQ ID NO: 37)
WASTRES
(SEQ ID NO: 38)
QQSDSLPITF.

In some embodiments, the polypeptides described herein are polypeptides comprising one or more of the VL chain and/or VH regions of antibodies that bind to the desired target, such as but not limited to EpCAM. In some embodiments, the polypeptide comprises one or more of the VL chain and/or VH chain CDRs of antibodies that bind to the desired target, such as but not limited to EpCAM. In some embodiments, the polypeptide comprises three CDRs of the VL chain and/or VH chain of the antibody. In some embodiments, the polypeptide comprises an amino acid sequence of the antibody that has any of the following: at least 5 contiguous amino acids of a sequence of an antibody that binds EpCAM, at least 8 contiguous amino acids of an antibody that binds EpCAM, at least about 10 contiguous amino acids of an antibody that binds EpCAM, at least about 15 contiguous amino acids of an antibody that binds EpCAM, at least about 20 contiguous amino acids of an antibody that binds EpCAM, at least about 25 contiguous amino acids of an antibody that binds EpCAM, at least about 30 contiguous amino acids of an antibody that binds EpCAM. In another embodiment, the 5 (or more) contiguous amino acids are from a CDR of the antibody.

In some embodiments, described herein is a bispecific molecule comprising an antigen binding region comprising a heavy chain variable region (VH) having at least 80% identity to amino acid sequence SEQ ID NO: 27.

In some embodiments, described herein is a bispecific molecule comprising an antigen binding region comprising a heavy chain variable region (VH) having at least 90% identity to amino acid sequence SEQ ID NO: 27.

In some embodiments, described herein is a bispecific molecule comprising an antigen binding region comprising a heavy chain variable region (VH) having at least 95% identity to amino acid sequence SEQ ID NO: 27.

In some embodiments, described herein is a bispecific molecule comprising an antigen binding region comprising a heavy chain variable region (VH) having at least 99% identity to amino acid sequence SEQ ID NO: 27.

In some embodiments, described herein is a bispecific molecule comprising an antigen binding region comprising a heavy chain variable region (VH) having 100% identity to amino acid sequence SEQ ID NO: 27. In some embodiments, the bispecific molecule further comprises a light chain variable region (VL) having at least 80% identity to amino acid sequence SEQ ID NO: 28. In some embodiments, the bispecific molecule further comprises a light chain variable region (VL) having at least 90% identity to amino acid sequence SEQ ID NO: 28. In some embodiments, the bispecific molecule further comprises a light chain variable region (VL) having at least 95% identity to amino acid sequence SEQ ID NO: 28. In some embodiments, the bispecific molecule further comprises a light chain variable region (VL) having at least 99% identity to amino acid sequence SEQ ID NO: 28. In some embodiments, the bispecific molecule further comprises a light chain variable region (VL) having 100% identity to amino acid sequence SEQ ID NO: 28.

In some embodiments, described herein is a bispecific molecule comprising an antigen binding region comprising a light chain variable region (VL) having at least 80% identity to amino acid sequence SEQ ID NO: 28.

In some embodiments, described herein is a bispecific molecule comprising an antigen binding region comprising a light chain variable region (VL) having at least 90% identity to amino acid sequence SEQ ID NO: 28.

In some embodiments, described herein is a bispecific molecule comprising an antigen binding region comprising a light chain variable region (VL) having at least 95% identity to amino acid sequence SEQ ID NO: 28.

In some embodiments, described herein is a bispecific molecule comprising an antigen binding region comprising a light chain variable region (VL) having at least 99% identity to amino acid sequence SEQ ID NO: 28.

In some embodiments, described herein is a bispecific molecule comprising an antigen binding region comprising a light chain variable region (VL) having 100% identity to amino acid sequence SEQ ID NO: 28. In some embodiments, the bispecific molecule further comprises a heavy chain variable region (VH) having at least 80% identity to amino acid sequence SEQ ID NO: 27. In some embodiments, the bispecific molecule further comprises a heavy chain variable region (VH) having at least 90% identity to amino acid sequence SEQ ID NO: 27. In some embodiments, the bispecific molecule further comprises a heavy chain variable region (VH) having at least 95% identity to amino acid sequence SEQ ID NO: 27. In some embodiments, the bispecific molecule further comprises a heavy chain variable region (VH) having at least 99% identity to amino acid sequence SEQ ID NO: 27. In some embodiments, the bispecific molecule further comprises a heavy chain variable region (VH) having 100% identity to amino acid sequence SEQ ID NO: 27.

In some embodiments, described herein is a bispecific molecule comprising a heavy chain variable region (VH) having at least 80% identity to amino acid sequence SEQ ID NO: 27 and a light chain variable region (VL) having at least 80% identity to amino acid sequence SEQ ID NO: 28. In some embodiments, the bispecific molecule comprises the light chain variable region (VL) having at least 85% identity to amino acid sequence SEQ ID NO: 28. In some embodiments, the bispecific molecule comprises the light chain variable region (VL) having at least 90% identity to amino acid sequence SEQ ID NO: 28. In some embodiments, the bispecific molecule comprises the light chain variable region (VL) having at least 95% identity to amino acid sequence SEQ ID NO: 28. In some embodiments, the bispecific molecule comprises the light chain variable region (VL) having at least 99% identity to amino acid sequence SEQ ID NO: 28. In some embodiments, the bispecific molecule comprises the light chain variable region (VL) having 100% identity to amino acid sequence SEQ ID NO: 28. In some embodiments, the bispecific molecule comprises the heavy chain variable region (VH) having at least 85% identity to amino acid sequence SEQ ID NO: 27. In some embodiments, the bispecific molecule comprises the heavy chain variable region (VH) having at least 90% identity to amino acid sequence SEQ ID NO: 27. In some embodiments, the bispecific molecule comprises the heavy chain variable region (VH) having at least 95% identity to amino acid sequence SEQ ID NO: 27. In some embodiments, the bispecific molecule comprises the heavy chain variable region (VH) having at least 99% identity to amino acid sequence SEQ ID NO: 27. In some embodiments, the bispecific molecule comprises the heavy chain variable region (VH) having at least 100% identity to amino acid sequence SEQ ID NO: 27.

In some embodiments, described herein is a bispecific molecule comprising a heavy chain sequence comprising a complementarity determining region (CDR) H1 having at least 80% identity to amino acid sequence SEQ ID NO: 33, a CDR H2 having at least 80% identity to amino acid sequence SEQ ID NO: 34, and a CDR H3 having at least 80% identity to amino acid sequence SEQ ID NO: 35, and a light chain sequence comprising a CDR L1 having at least 80% identity to amino acid sequence SEQ ID NO: 36, a CDR L2 having at least 80% identity to amino acid sequence SEQ ID NO: 37, and a CDR L3 having at least 80% identity to amino acid sequence SEQ ID NO: 38. In some embodiments, the bispecific molecule comprises the light chain sequence comprising the CDR L1 having at least 85% identity to amino acid sequence SEQ ID NO: 36, the CDR L2 having at least 85% identity to amino acid sequence SEQ ID NO: 37, and the CDR L3 having at least 85% identity to amino acid sequence SEQ ID NO: 38. In some embodiments, the bispecific molecule comprises the light chain sequence comprising the CDR L1 having at least 90% identity to amino acid sequence SEQ ID NO: 36, the CDR L2 having at least 90% identity to amino acid sequence SEQ ID NO: 37, and the CDR L3 having at least 90% identity to amino acid sequence SEQ ID NO: 38. In some embodiments, the bispecific molecule comprises light chain sequence comprising the CDR L1 having at least 95% identity to amino acid sequence SEQ ID NO: 36, the CDR L2 having at least 95% identity to amino acid sequence SEQ ID NO: 37, and the CDR L3 having at least 95% identity to amino acid sequence SEQ ID NO: 38. In some embodiments, the bispecific molecule comprises the light chain sequence comprising the CDR L1 having at least 99% identity to amino acid sequence SEQ ID NO: 36, the CDR L2 having at least 99% identity to amino acid sequence SEQ ID NO: 37, and the CDR L3 having at least 99% identity to amino acid sequence SEQ ID NO: 38. In some embodiments, the bispecific molecule comprises the light chain sequence comprising the CDR L1 having 100% identity to amino acid sequence SEQ ID NO: 36, the CDR L2 having 100% identity to amino acid sequence SEQ ID NO: 37, and the CDR L3 having at least 100% identity to amino acid sequence SEQ ID NO: 38. In some embodiments, the bispecific molecule comprises the heavy chain sequence comprising the CDR H1 having at least 85% identity to amino acid sequence SEQ ID NO: 33, the CDR H2 having at least 85% identity to amino acid sequence SEQ ID NO: 34, and the CDR H3 having at least 85% identity to amino acid sequence SEQ ID NO: 35. In some embodiments, the bispecific molecule comprises the heavy chain sequence comprising a the CDR H1 having at least 90% identity to amino acid sequence SEQ ID NO: 33, the CDR H2 having at least 90% identity to amino acid sequence SEQ ID NO: 34, and the CDR H3 having at least 90% identity to amino acid sequence SEQ ID NO: 35. In some embodiments, the bispecific molecule comprises the heavy chain sequence comprising a the CDR H1 having at least 95% identity to amino acid sequence SEQ ID NO: 33, the CDR H2 having at least 95% identity to amino acid sequence SEQ ID NO: 34, and the CDR H3 having at least 95% identity to amino acid sequence SEQ ID NO: 35. In some embodiments, the bispecific molecule comprises the heavy chain sequence comprising a the CDR H1 having at least 99% identity to amino acid sequence SEQ ID NO: 33, the CDR H2 having at least 99% identity to amino acid sequence SEQ ID NO: 34, and the CDR H3 having at least 99% identity to amino acid sequence SEQ ID NO: 35. In some embodiments, the bispecific molecule comprises the heavy chain sequence comprising a the CDR H1 having at least 100% identity to amino acid sequence SEQ ID NO: 33, the CDR H2 having at least 100% identity to amino acid sequence SEQ ID NO: 34, and the CDR H3 having at least 100% identity to amino acid sequence SEQ ID NO: 35.

In some embodiments, described herein is a bispecific molecule that binds to EpCAM comprising at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 36, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 37, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 38. In some embodiments, bispecific molecules that bind to EpCAM comprise at least one of a light chain CDR1 having an amino acid sequence 100% identical to an amino acid sequence set forth as SEQ ID NO: 36, a light chain CDR2 having an amino acid sequence 100% identical to an amino acid sequence set forth as SEQ ID NO: 37, and a light chain CDR3 having an amino acid sequence 100% identical to an amino acid sequence set forth as SEQ ID NO: 38.

In some embodiments, described herein is a bispecific molecule that binds to EpCAM comprising at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 33, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 34, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 35. In some embodiments, bispecific molecules that bind to EpCAM comprise at least one of a heavy chain CDR1 having an amino acid sequence 100% identical to an amino acid sequence set forth as SEQ ID NO: 33, a heavy chain CDR2 having an amino acid sequence 100% identical to an amino acid sequence set forth as SEQ ID NO: 34, and a heavy chain CDR3 having an amino acid sequence 100% identical to an amino acid sequence set forth as SEQ ID NO: 35.

In some embodiments, described herein is a bispecific molecule that binds to EpCAM comprising at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 36, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 37, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 38, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 33, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 34, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 35. In some embodiments, bispecific molecules that bind to EpCAM comprise at least one of a light chain CDR1 having an amino acid sequence 100% identical to an amino acid sequence set forth as SEQ ID NO: 36, a light chain CDR2 having an amino acid sequence 100% identical to an amino acid sequence set forth as SEQ ID NO: 37, a light chain CDR3 having an amino acid sequence 100% identical to an amino acid sequence set forth as SEQ ID NO: 38, a heavy chain CDR1 having an amino acid sequence 100% identical to an amino acid sequence set forth as SEQ ID NO: 33, a heavy chain CDR2 having an amino acid sequence 100% identical to an amino acid sequence set forth as SEQ ID NO: 34, and a heavy chain CDR3 having an amino acid sequence 100% identical to an amino acid sequence set forth as SEQ ID NO: 35.

In some embodiments, described herein is a bispecific molecule comprising a VL and VH chain that confers binding specificity to EpCAM wherein amino acids in the framework can be varied. In some embodiments, amino acid variation involves introduction of conservative amino acid substitutions. In some embodiments, bispecific molecules that bind to EpCAM comprise a light chain variable (VL) region and a heavy chain variable (VH) region, wherein the VL region comprises a light chain CDR1 (CDRL1) having an amino acid sequence 100% identical to an amino acid sequence set forth as SEQ ID NO: 36, a light chain CDR2 (CDRL2) having an amino acid sequence 100% identical to an amino acid sequence set forth as SEQ ID NO: 37, a light chain CDR3 (CDRL3) having an amino acid sequence 100% identical to an amino acid sequence set forth as SEQ ID NO: 38 wherein the amino acid sequence of the VL region outside of CDRL1, CDRL2, and CDRL3 has an overall sequence identity of at least 75%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 28, and wherein the VH region comprises a heavy chain CDR1 (CDRH1) having an amino acid sequence 100% identical to an amino acid sequence set forth as SEQ ID NO: 33, a heavy chain CDR2 (CDRH2) having an amino acid sequence 100% identical to an amino acid sequence set forth as SEQ ID NO: 34, a heavy chain CDR3 (CDRH3) having an amino acid sequence 100% identical to an amino acid sequence set forth as SEQ ID NO: 35 wherein the amino acid sequence of the VH region outside of CDRH1, CDRH2, and CDRH3 has an overall sequence identity of at least 75%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 27. In some embodiments, the amino acid sequence of the VL region outside of CDRL1, CDRL2, and CDRL3 has an overall sequence identity of at least 90%, identity to SEQ ID NO: 28 and the amino acid sequence of the VH region outside of CDRH1, CDRH2, and CDRH3 has an overall sequence identity of at least 90% identity to SEQ ID NO: 27. In some embodiments, the amino acid sequence of the VL region outside of CDRL1, CDRL2, and CDRL3 has an overall sequence identity of at least 95%, identity to SEQ ID NO: 28 and the amino acid sequence of the VH region outside of CDRH1, CDRH2, and CDRH3 has an overall sequence identity of at least 95% identity to SEQ ID NO: 27. In some embodiments, the amino acid sequence of the VL region outside of CDRL1, CDRL2, and CDRL3 has an overall sequence identity of at least 99%, identity to SEQ ID NO: 28 and the amino acid sequence of the VH region outside of CDRH1, CDRH2, and CDRH3 has an overall sequence identity of at least 99% identity to SEQ ID NO: 27.

In some embodiments, bispecific molecules that bind to EpCAM comprise a light chain variable (VL) region and a heavy chain variable (VH) region, wherein the VL region comprises a light chain CDR1 (CDRL1) having an amino acid sequence 100% identical to an amino acid sequence set forth as SEQ ID NO: 36, a light chain CDR2 (CDRL2) having an amino acid sequence 100% identical to an amino acid sequence set forth as SEQ ID NO: 37, a light chain CDR3 (CDRL3) having an amino acid sequence 100% identical to an amino acid sequence set forth as SEQ ID NO: 38 wherein the amino acid sequence of the VL region outside of CDRL1, CDRL2, and CDRL3 has an overall sequence identity of at least 75%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 28 and wherein the V gene usage of the VL region is a kappa light chain, and wherein the VH region comprises a heavy chain CDR1 (CDRH1) having an amino acid sequence 100% identical to an amino acid sequence set forth as SEQ ID NO: 33, a heavy chain CDR2 (CDRH2) having an amino acid sequence 100% identical to an amino acid sequence set forth as SEQ ID NO: 34, a heavy chain CDR3 (CDRH3) having an amino acid sequence 100% identical to an amino acid sequence set forth as SEQ ID NO: 35 wherein the amino acid sequence of the VH region outside of CDRH1, CDRH2, and CDRH3 has an overall sequence identity of at least 75%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 27 and wherein the V gene usage of the VH region is IGHV gene. In some embodiments, the amino acid sequence of the VL region outside of CDRL1, CDRL2, and CDRL3 has an overall sequence identity of at least 90%, identity to SEQ ID NO: 28 and the amino acid sequence of the VH region outside of CDRH1, CDRH2, and CDRH3 has an overall sequence identity of at least 90% identity to SEQ ID NO: 27. In some embodiments, the amino acid sequence of the VL region outside of CDRL1, CDRL2, and CDRL3 has an overall sequence identity of at least 95%, identity to SEQ ID NO: 28 and the amino acid sequence of the VH region outside of CDRH1, CDRH2, and CDRH3 has an overall sequence identity of at least 95% identity to SEQ ID NO: 27. In some embodiments, the amino acid sequence of the VL region outside of CDRL1, CDRL2, and CDRL3 has an overall sequence identity of at least 99%, identity to SEQ ID NO: 28 and the amino acid sequence of the VH region outside of CDRH1, CDRH2, and CDRH3 has an overall sequence identity of at least 99% identity to SEQ ID NO: 27. In some embodiments, the V gene usage of the VL region is a kappa 5.1 light chain.

### Antigen Binding Regions or Domains Specific for CD163

Provided below is the amino acid sequence corresponding to the CD163 (Uniport ID: Q86VB7) sequence:

(SEQ ID NO: 6)
MSKLRMVLLEDSGSADFRRHFVNLSPFTITVVLLLSACFVTSSLGGTDK
ELRLVDGENKCSGRVEVKVQEEWGTVCNNGWSMEAVSVICNQLGCPTAI
KAPGWANSSAGSGRIWMDHVSCRGNESALWDCKHDGWGKHSNCTHQQDA
GVTCSDGSNLEMRLTRGGNMCSGRIEIKFQGRWGTVCDDNFNIDHASVI
CRQLECGSAVSFSGSSNFGEGSGPIWFDDLICNGNESALWNCKHQGWGK
HNCDHAEDAGVICSKGADLSLRLVDGVTECSGRLEVRFQGEWGTICDDG
WDSYDAAVACKQLGCPTAVTAIGRVNASKGFGHIWLDSVSCQGHEPAIW
QCKHHEWGKHYCNHNEDAGVTCSDGSDLELRLRGGGSRCAGTVEVEIQR
LLGKVCDRGWGLKEADVVCRQLGCGSALKTSYQVYSKIQATNTWLFLSS
CNGNETSLWDCKNWQWGGLTCDHYEEAKITCSAHREPRLVGGDIPCSGR
VEVKHGDTWGSICDSDFSLEAASVLCRELQCGTVVSILGGAHFGEGNGQ
IWAEEFQCEGHESHLSLCPVAPRPEGTCSHSRDVGVVCSRYTEIRLVNG
KTPCEGRVELKTLGAWGSLCNSHWDIEDAHVLCQQLKCGVALSTPGGAR
FGKGNGQIWRHMFHCTGTEQHMGDCPVTALGASLCPSEQVASVICSGNQ
SQTLSSCNSSSLGPTRPTIPEESAVACIESGQLRLVNGGGRCAGRVEIY
HEGSWGTICDDSWDLSDAHVVCRQLGCGEAINATGSAHFGEGTGPIWLD
EMKCNGKESRIWQCHSHGWGQQNCRHKEDAGVICSEFMSLRLTSEASRE
ACAGRLEVFYNGAWGTVGKSSMSETTVGVVCRQLGCADKGKINPASLDK
AMSIPMWVDNVQCPKGPDTLWQCPSSPWEKRLASPSEETWITCDNKIRL
QEGPTSCSGRVEIWHGGSWGTVCDDSWDLDDAQVVCQQLGCGPALKAFK
EAEFGQGTGPIWLNEVKCKGNESSLWDCPARRWGHSECGHKEDAAVNCT
DISVQKTPQKATTGRSSRQSSFIAVGILGVVLLAIFVALFFLTKKRRQR
QRLAVSSRGENLVHQIQYREMNSCLNADDLDLMNSSENSHESADFSAAE
LISVSKFLPISGMEKEAILSHTEKENGNL

In some embodiments, one of the antigen binding regions of the bispecific molecule binds to CD163. In some embodiments, one of the antigen binding regions of the bispecific molecule binds to an epitope of SEQ ID NO:6. In some embodiments the invention relates to IgG-based structures, for example a polynucleotide and polypeptide sequence encoding a heavy chain and a light chain sequence of an CD163-specific antibody. Embodiments and sequences of antibody molecules that specifically bind CD163 are known in the art, including those disclosed in United States Patent Application Publication No.: US 2017/0119790 A1, the content of which is hereby incorporated by reference in its entirety.

In some embodiments, the polypeptides described herein are polypeptides comprising one or more of the VL chain and/or VH regions of antibodies that bind to the desired target, such as but not limited to CD163. In some embodiments, the polypeptide comprises one or more of the VL chain and/or VH chain CDRs of antibodies that bind to the desired target, such as but not limited to CD163. In some embodiments, the polypeptide comprises three CDRs of the VL chain and/or VH chain of the antibody. In some embodiments, the polypeptide comprises an amino acid sequence of the antibody that has any of the following: at least 5 contiguous amino acids of a sequence of an antibody that binds CD163, at least 8 contiguous amino acids of an antibody that binds CD163, at least about 10 contiguous amino acids of an antibody that binds CD163, at least about 15 contiguous amino acids of an antibody that binds CD163, at least about 20 contiguous amino acids of an antibody that binds CD163, at least about 25 contiguous amino acids of an antibody that binds CD163, at least about 30 contiguous amino acids of an antibody that binds CD163. In another embodiment, the 5 (or more) contiguous amino acids are from a CDR of the antibody.

In some embodiments the antigen binding region specific to CD163 comprises the VH chain of SEQ ID NO: 29:

(SEQ ID NO: 29)
QVQLQESGPGLVKPSETLSLTCTVSGYSITSDYAWNWIRQFPGNKLEWM
GYITYSGSTYYNPSLKSRVTISVDTSKNQFSLKLSSVTAADTATYYCVS
GTYYFDYWGQGTTLTVSS.

The CDRs are shown in bold and underlined and comprise amino acids 26 to 33 (CDR1), 54 to 56 (CDR2), and 96 to 107 (CDR3).

In some embodiments, the antigen binding region specific to CD163 comprises an amino acid sequence having at least 60%, 65%, 70%, 75%, 80%, 85%, 90%, 91%, 92%, 93%, 94%, 95%, 96%, 97%, 98%, or 99% identity with SEQ ID NO: 29.

In some embodiments, the antigen binding region specific to CD163 comprises a VH region comprising one or more CDR sequences selected from:

(SEQ ID NO: 39)
GYSITSDY
(SEQ ID NO: 40)
YSG
(SEQ ID NO: 41)
CVSGTYYFDYWG.

In some embodiments the antigen binding region specific to CD163 comprises the VL chain of SEQ ID NO: 30:

(SEQ ID NO: 30)
DIVMTQSPSSLSASVGDRVTITCRASQSVSSDVAWFQQKPGKSPKPLIY
YASNRYSGVPSRFSGSGSGTDFTLTISSLQAEDFAVYFCGQDYTSPRTF
GGGTKLEIKR.

The CDRs are shown in bold and underlined and comprise amino acids 25 to 33 (CDR1), 50 to 52 (CDR2), and 90 to 97 (CDR3).

In some embodiments, the antigen binding region specific to CD163 comprises an amino acid sequence having at least 60%, 65%, 70%, 75%, 80%, 85%, 90%, 91%, 92%, 93%, 94%, 95%, 96%, 97%, 98%, or 99% identity with SEQ ID NO: 30.

In some embodiments, the antigen binding region specific to CD163 comprises a VL region comprising one or more CDR sequences selected from:

(SEQ ID NO: 42)
ASQSVSSDV
(SEQ ID NO: 43)
YAS
(SEQ ID NO: 44)
QDYTSPRT.

In some embodiments, described herein is a bispecific molecule comprising an antigen binding region comprising a heavy chain variable region (VH) having at least 80% identity to amino acid sequence SEQ ID NO: 29.

In some embodiments, described herein is a bispecific molecule comprising an antigen binding region comprising a heavy chain variable region (VH) having at least 90% identity to amino acid sequence SEQ ID NO: 29.

In some embodiments, described herein is a bispecific molecule comprising an antigen binding region comprising a heavy chain variable region (VH) having at least 95% identity to amino acid sequence SEQ ID NO: 29.

In some embodiments, described herein is a bispecific molecule comprising an antigen binding region comprising a heavy chain variable region (VH) having at least 99% identity to amino acid sequence SEQ ID NO: 29.

In some embodiments, described herein is a bispecific molecule comprising an antigen binding region comprising a heavy chain variable region (VH) having 100% identity to amino acid sequence SEQ ID NO: 29. In some embodiments, the bispecific molecule further comprises a light chain variable region (VL) having at least 80% identity to amino acid sequence SEQ ID NO: 30. In some embodiments, the bispecific molecule further comprises a light chain variable region (VL) having at least 90% identity to amino acid sequence SEQ ID NO: 30. In some embodiments, the bispecific molecule further comprises a light chain variable region (VL) having at least 95% identity to amino acid sequence SEQ ID NO: 30. In some embodiments, the bispecific molecule further comprises a light chain variable region (VL) having at least 99% identity to amino acid sequence SEQ ID NO: 30. In some embodiments, the bispecific molecule further comprises a light chain variable region (VL) having 100% identity to amino acid sequence SEQ ID NO: 30.

In some embodiments, described herein is a bispecific molecule comprising an antigen binding region comprising a light chain variable region (VL) having at least 80% identity to amino acid sequence SEQ ID NO: 30.

In some embodiments, described herein is a bispecific molecule comprising an antigen binding region comprising a light chain variable region (VL) having at least 90% identity to amino acid sequence SEQ ID NO: 30.

In some embodiments, described herein is a bispecific molecule comprising an antigen binding region comprising a light chain variable region (VL) having at least 95% identity to amino acid sequence SEQ ID NO: 30.

In some embodiments, described herein is a bispecific molecule comprising an antigen binding region comprising a light chain variable region (VL) having at least 99% identity to amino acid sequence SEQ ID NO: 30.

In some embodiments, described herein is a bispecific molecule comprising an antigen binding region comprising a light chain variable region (VL) having 100% identity to amino acid sequence SEQ ID NO: 30. In some embodiments, the bispecific molecule further comprises a heavy chain variable region (VH) having at least 80% identity to amino acid sequence SEQ ID NO: 29. In some embodiments, the bispecific molecule further comprises a heavy chain variable region (VH) having at least 90% identity to amino acid sequence SEQ ID NO: 29. In some embodiments, the bispecific molecule further comprises a heavy chain variable region (VH) having at least 95% identity to amino acid sequence SEQ ID NO: 29. In some embodiments, the bispecific molecule further comprises a heavy chain variable region (VH) having at least 99% identity to amino acid sequence SEQ ID NO: 29. In some embodiments, the bispecific molecule further comprises a heavy chain variable region (VH) having 100% identity to amino acid sequence SEQ ID NO: 29.

In some embodiments, described herein is a bispecific molecule comprising a heavy chain variable region (VH) having at least 80% identity to amino acid sequence SEQ ID NO: 29 and a light chain variable region (VL) having at least 80% identity to amino acid sequence SEQ ID NO: 30. In some embodiments, the bispecific molecule comprises the light chain variable region (VL) having at least 85% identity to amino acid sequence SEQ ID NO: 30. In some embodiments, the bispecific molecule comprises the light chain variable region (VL) having at least 90% identity to amino acid sequence SEQ ID NO: 30. In some embodiments, the bispecific molecule comprises the light chain variable region (VL) having at least 95% identity to amino acid sequence SEQ ID NO: 30. In some embodiments, the bispecific molecule comprises the light chain variable region (VL) having at least 99% identity to amino acid sequence SEQ ID NO: 30. In some embodiments, the bispecific molecule comprises the light chain variable region (VL) having 100% identity to amino acid sequence SEQ ID NO: 30. In some embodiments, the bispecific molecule comprises the heavy chain variable region (VH) having at least 85% identity to amino acid sequence SEQ ID NO: 29. In some embodiments, the bispecific molecule comprises the heavy chain variable region (VH) having at least 90% identity to amino acid sequence SEQ ID NO: 29. In some embodiments, the bispecific molecule comprises the heavy chain variable region (VH) having at least 95% identity to amino acid sequence SEQ ID NO: 29. In some embodiments, the bispecific molecule comprises the heavy chain variable region (VH) having at least 99% identity to amino acid sequence SEQ ID NO: 29. In some embodiments, the bispecific molecule comprises the heavy chain variable region (VH) having at least 100% identity to amino acid sequence SEQ ID NO: 29.

In some embodiments, described herein is a bispecific molecule comprising a heavy chain sequence comprising a complementarity determining region (CDR) H1 having at least 80% identity to amino acid sequence SEQ ID NO: 39, a CDR H2 having at least 80% identity to amino acid sequence SEQ ID NO: 40, and a CDR H3 having at least 80% identity to amino acid sequence SEQ ID NO: 41, and a light chain sequence comprising a CDR L1 having at least 80% identity to amino acid sequence SEQ ID NO: 42, a CDR L2 having at least 80% identity to amino acid sequence SEQ ID NO: 43, and a CDR L3 having at least 80% identity to amino acid sequence SEQ ID NO: 44. In some embodiments, the bispecific molecule comprises the light chain sequence comprising the CDR L1 having at least 85% identity to amino acid sequence SEQ ID NO: 42, the CDR L2 having at least 85% identity to amino acid sequence SEQ ID NO: 43, and the CDR L3 having at least 85% identity to amino acid sequence SEQ ID NO: 44. In some embodiments, the bispecific molecule comprises the light chain sequence comprising the CDR L1 having at least 90% identity to amino acid sequence SEQ ID NO: 42, the CDR L2 having at least 90% identity to amino acid sequence SEQ ID NO: 43, and the CDR L3 having at least 90% identity to amino acid sequence SEQ ID NO: 44. In some embodiments, the bispecific molecule comprises light chain sequence comprising the CDR L1 having at least 95% identity to amino acid sequence SEQ ID NO: 42, the CDR L2 having at least 95% identity to amino acid sequence SEQ ID NO: 43, and the CDR L3 having at least 95% identity to amino acid sequence SEQ ID NO: 44. In some embodiments, the bispecific molecule comprises the light chain sequence comprising the CDR L1 having at least 99% identity to amino acid sequence SEQ ID NO: 42, the CDR L2 having at least 99% identity to amino acid sequence SEQ ID NO: 43, and the CDR L3 having at least 99% identity to amino acid sequence SEQ ID NO: 44. In some embodiments, the bispecific molecule comprises the light chain sequence comprising the CDR L1 having 100% identity to amino acid sequence SEQ ID NO: 42, the CDR L2 having 100% identity to amino acid sequence SEQ ID NO: 43, and the CDR L3 having at least 100% identity to amino acid sequence SEQ ID NO: 44. In some embodiments, the bispecific molecule comprises the heavy chain sequence comprising the CDR H1 having at least 85% identity to amino acid sequence SEQ ID NO: 39, the CDR H2 having at least 85% identity to amino acid sequence SEQ ID NO: 40, and the CDR H3 having at least 85% identity to amino acid sequence SEQ ID NO: 41. In some embodiments, the bispecific molecule comprises the heavy chain sequence comprising a the CDR H1 having at least 90% identity to amino acid sequence SEQ ID NO: 39, the CDR H2 having at least 90% identity to amino acid sequence SEQ ID NO: 40, and the CDR H3 having at least 90% identity to amino acid sequence SEQ ID NO: 41. In some embodiments, the bispecific molecule comprises the heavy chain sequence comprising a the CDR H1 having at least 95% identity to amino acid sequence SEQ ID NO: 39, the CDR H2 having at least 95% identity to amino acid sequence SEQ ID NO: 40, and the CDR H3 having at least 95% identity to amino acid sequence SEQ ID NO: 41. In some embodiments, the bispecific molecule comprises the heavy chain sequence comprising a the CDR H1 having at least 99% identity to amino acid sequence SEQ ID NO: 39, the CDR H2 having at least 99% identity to amino acid sequence SEQ ID NO: 40, and the CDR H3 having at least 99% identity to amino acid sequence SEQ ID NO: 41. In some embodiments, the bispecific molecule comprises the heavy chain sequence comprising a the CDR H1 having at least 100% identity to amino acid sequence SEQ ID NO: 39, the CDR H2 having at least 100% identity to amino acid sequence SEQ ID NO: 40, and the CDR H3 having at least 100% identity to amino acid sequence SEQ ID NO: 41.

In some embodiments, described herein is a bispecific molecule that binds to CD163 comprising at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 42, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 43, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 44. In some embodiments, bispecific molecules that bind to CD163 comprise at least one of a light chain CDR1 having an amino acid sequence 100% identical to an amino acid sequence set forth as SEQ ID NO: 42, a light chain CDR2 having an amino acid sequence 100% identical to an amino acid sequence set forth as SEQ ID NO: 43, and a light chain CDR3 having an amino acid sequence 100% identical to an amino acid sequence set forth as SEQ ID NO: 44.

In some embodiments, described herein is a bispecific molecule that binds to CD163 comprising at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 39, a heavy chain CDR2 having an amino acid sequence at least about 75%, 80%, 81%, 82%, 83%8, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 40, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 41. In some embodiments, bispecific molecules that bind to CD163 comprise at least one of a heavy chain CDR1 having an amino acid sequence 100% identical to an amino acid sequence set forth as SEQ ID NO: 39, a heavy chain CDR2 having an amino acid sequence 100% identical to an amino acid sequence set forth as SEQ ID NO: 40, and a heavy chain CDR3 having an amino acid sequence 100% identical to an amino acid sequence set forth as SEQ ID NO: 41.

In some embodiments, described herein is a bispecific molecule that binds to CD163 comprising at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 42, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 43, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 44, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 39, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 40, and a heavy chain CDR3 having an amino acid sequence at least about 75%, 80%, 81%, 82%, 83%, 84%, 85%, 86%, 87%, 88%, 89%0, 90%, 91%, 92%, 93%, 94%, 95%, 96%, 97%, 98%, or 99% identical to an amino acid sequence set forth as SEQ ID NO: 41. In some embodiments, bispecific molecules that bind to CD163 comprise at least one of a light chain CDR1 having an amino acid sequence 100% identical to an amino acid sequence set forth as SEQ ID NO: 42, a light chain CDR2 having an amino acid sequence 100% identical to an amino acid sequence set forth as SEQ ID NO: 43, a light chain CDR3 having an amino acid sequence 100% identical to an amino acid sequence set forth as SEQ ID NO: 44, a heavy chain CDR1 having an amino acid sequence 100% identical to an amino acid sequence set forth as SEQ ID NO: 39, a heavy chain CDR2 having an amino acid sequence 100% identical to an amino acid sequence set forth as SEQ ID NO: 40, and a heavy chain CDR3 having an amino acid sequence 100% identical to an amino acid sequence set forth as SEQ ID NO: 41.

In some embodiments, described herein is a bispecific molecule comprising a VL and VH chain that confers binding specificity to CD163 wherein amino acids in the framework can be varied. In some embodiments, amino acid variation involves introduction of conservative amino acid substitutions. In some embodiments, bispecific molecules that bind to CD163 comprise a light chain variable (VL) region and a heavy chain variable (VH) region, wherein the VL region comprises a light chain CDR1 (CDRL1) having an amino acid sequence 100% identical to an amino acid sequence set forth as SEQ ID NO: 42, a light chain CDR2 (CDRL2) having an amino acid sequence 100% identical to an amino acid sequence set forth as SEQ ID NO: 43, a light chain CDR3 (CDRL3) having an amino acid sequence 100% identical to an amino acid sequence set forth as SEQ ID NO: 44 wherein the amino acid sequence of the VL region outside of CDRL1, CDRL2, and CDRL3 has an overall sequence identity of at least 75%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 30, and wherein the VH region comprises a heavy chain CDR1 (CDRH1) having an amino acid sequence 100% identical to an amino acid sequence set forth as SEQ ID NO: 39, a heavy chain CDR2 (CDRH2) having an amino acid sequence 100% identical to an amino acid sequence set forth as SEQ ID NO: 40, a heavy chain CDR3 (CDRH3) having an amino acid sequence 100% identical to an amino acid sequence set forth as SEQ ID NO: 41 wherein the amino acid sequence of the VH region outside of CDRH1, CDRH2, and CDRH3 has an overall sequence identity of at least 75%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 29. In some embodiments, the amino acid sequence of the VL region outside of CDRL1, CDRL2, and CDRL3 has an overall sequence identity of at least 90%, identity to SEQ ID NO: 30 and the amino acid sequence of the VH region outside of CDRH1, CDRH2, and CDRH3 has an overall sequence identity of at least 90% identity to SEQ ID NO: 29. In some embodiments, the amino acid sequence of the VL region outside of CDRL1, CDRL2, and CDRL3 has an overall sequence identity of at least 95%, identity to SEQ ID NO: 30 and the amino acid sequence of the VH region outside of CDRH1, CDRH2, and CDRH3 has an overall sequence identity of at least 95% identity to SEQ ID NO: 29. In some embodiments, the amino acid sequence of the VL region outside of CDRL1, CDRL2, and CDRL3 has an overall sequence identity of at least 99%, identity to SEQ ID NO: 30 and the amino acid sequence of the VH region outside of CDRH1, CDRH2, and CDRH3 has an overall sequence identity of at least 99% identity to SEQ ID NO: 29.

In some embodiments, bispecific molecules that bind to CD163 comprise a light chain variable (VL) region and a heavy chain variable (VH) region, wherein the VL region comprises a light chain CDR1 (CDRL1) having an amino acid sequence 100% identical to an amino acid sequence set forth as SEQ ID NO: 42, a light chain CDR2 (CDRL2) having an amino acid sequence 100% identical to an amino acid sequence set forth as SEQ ID NO: 43, a light chain CDR3 (CDRL3) having an amino acid sequence 100% identical to an amino acid sequence set forth as SEQ ID NO: 44 wherein the amino acid sequence of the VL region outside of CDRL1, CDRL2, and CDRL3 has an overall sequence identity of at least 75%, 80%, 81%, 82%, 83%8, 84%, 85%, 86%, 87%, 88%, 89%, 90%, 91%, 92%, 93%, 94%, 95%, 96%, 97%, 98%, or 99% identity to SEQ ID NO: 30 and wherein the V gene usage of the VL region is a kappa light chain, and wherein the VH region comprises a heavy chain CDR1 (CDRH1) having an amino acid sequence 100% identical to an amino acid sequence set forth as SEQ ID NO: 39, a heavy chain CDR2 (CDRH2) having an amino acid sequence 100% identical to an amino acid sequence set forth as SEQ ID NO: 40, a heavy chain CDR3 (CDRH3) having an amino acid sequence 100% identical to an amino acid sequence set forth as SEQ ID NO: 41 wherein the amino acid sequence of the VH region outside of CDRH1, CDRH2, and CDRH3 has an overall sequence identity of at least 75%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 29 and wherein the V gene usage of the VH region is IGHV gene. In some embodiments, the amino acid sequence of the VL region outside of CDRL1, CDRL2, and CDRL3 has an overall sequence identity of at least 90%, identity to SEQ ID NO: 30 and the amino acid sequence of the VH region outside of CDRH1, CDRH2, and CDRH3 has an overall sequence identity of at least 90% identity to SEQ ID NO: 29. In some embodiments, the amino acid sequence of the VL region outside of CDRL1, CDRL2, and CDRL3 has an overall sequence identity of at least 95%, identity to SEQ ID NO: 30 and the amino acid sequence of the VH region outside of CDRH1, CDRH2, and CDRH3 has an overall sequence identity of at least 95% identity to SEQ ID NO: 29. In some embodiments, the amino acid sequence of the VL region outside of CDRL1, CDRL2, and CDRL3 has an overall sequence identity of at least 99%, identity to SEQ ID NO: 30 and the amino acid sequence of the VH region outside of CDRH1, CDRH2, and CDRH3 has an overall sequence identity of at least 99% identity to SEQ ID NO: 29. In some embodiments, the V gene usage of the VL region is a kappa 8 light chain. In some embodiments, the V gene usage of the VL region is IGKV1D-39*01. In some embodiments, the V gene usage of the VH region is a IGHV4 gene. In some embodiments, the V gene usage of the VH region is IGHV4-b*01.

In some embodiments the antigen binding region specific to CD163 comprises the VH chain of SEQ ID NO: 31:

(SEQ ID NO: 31)
EVQLVESGGGVVQPGRSLRLSCAASGFTFSSYAMHWVRQAPGKGLEWVA
VISYDGSNKYYADSVKGRFTISRDNSKNTLYLQMNSLRAEDTAVYYCAR
ENVRPYYDFWSGYYSEYYYYGMDVWGQGTTVTVSSA.

The CDRs are shown in bold and underlined and comprise amino acids 31 to 35 (CDR1), 50 to 65 (CDR2), and 99 to 122 (CDR3).

In some embodiments, the antigen binding region specific to CD163 comprises an amino acid sequence having at least 60%, 65%, 70%, 75%, 80%, 85%, 90%, 91%, 92%, 93%, 94%, 95%, 96%, 97%, 98%, or 99% identity with SEQ ID NO: 31.

In some embodiments, the antigen binding region specific to CD163 comprises a VH region comprising one or more CDR sequences selected from:

(SEQ ID NO: 45)
SYAMH
(SEQ ID NO: 46)
VISYDGSNKYYADSVK
(SEQ ID NO: 47)
ENVRPYYDFWSGYYSEYYYYGMDV.

In some embodiments the antigen binding region specific to CD163 comprises the VL chain of SEQ ID NO: 32:

(SEQ ID NO: 32)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPRGT
FGQGTKVEI.

The CDRs are shown in bold and underlined and comprise amino acids 24 to 34 (CDR1), 50 to 55 (CDR2), and 89 to 98 (CDR3).

In some embodiments, the antigen binding region specific to CD163 comprises an amino acid sequence having at least 60%, 65%, 70%, 75%, 80%, 85%, 90%, 91%, 92%, 93%, 94%, 95%, 96%, 97%, 98%, or 99% identity with SEQ ID NO: 32.

In some embodiments, the antigen binding region specific to CD163 comprises a VL region comprising one or more CDR sequences selected from:

(SEQ ID NO: 48)
RASQSISSYLN
(SEQ ID NO: 49)
AASSLQS
(SEQ ID NO: 50)
QQSYSTPRGT.

In some embodiments, described herein is a bispecific molecule comprising an antigen binding region comprising a heavy chain variable region (VH) having at least 80% identity to amino acid sequence SEQ ID NO: 31.

In some embodiments, described herein is a bispecific molecule comprising an antigen binding region comprising a heavy chain variable region (VH) having at least 90% identity to amino acid sequence SEQ ID NO: 31.

In some embodiments, described herein is a bispecific molecule comprising an antigen binding region comprising a heavy chain variable region (VH) having at least 95% identity to amino acid sequence SEQ ID NO: 31.

In some embodiments described herein, is a bispecific molecule comprising an antigen binding region comprising a heavy chain variable region (VH) having at least 99% identity to amino acid sequence SEQ ID NO: 31.

In some embodiments described herein, is a bispecific molecule comprising an antigen binding region comprising a heavy chain variable region (VH) having 100% identity to amino acid sequence SEQ ID NO: 31. In some embodiments, the bispecific molecule further comprises a light chain variable region (VL) having at least 80% identity to amino acid sequence SEQ ID NO: 32. In some embodiments, the bispecific molecule further comprises a light chain variable region (VL) having at least 90% identity to amino acid sequence SEQ ID NO: 32. In some embodiments, the bispecific molecule further comprises a light chain variable region (VL) having at least 95% identity to amino acid sequence SEQ ID NO: 32. In some embodiments, the bispecific molecule further comprises a light chain variable region (VL) having at least 99% identity to amino acid sequence SEQ ID NO: 32. In some embodiments, the bispecific molecule further comprises a light chain variable region (VL) having 100% identity to amino acid sequence SEQ ID NO: 32.

In some embodiments described herein, is a bispecific molecule comprising an antigen binding region comprising a light chain variable region (VL) having at least 80% identity to amino acid sequence SEQ ID NO: 32.

In some embodiments described herein, is a bispecific molecule comprising an antigen binding region comprising a light chain variable region (VL) having at least 90% identity to amino acid sequence SEQ ID NO: 32.

In some embodiments described herein, is a bispecific molecule comprising an antigen binding region comprising a light chain variable region (VL) having at least 95% identity to amino acid sequence SEQ ID NO: 32.

In some embodiments described herein, is a bispecific molecule comprising an antigen binding region comprising a light chain variable region (VL) having at least 99% identity to amino acid sequence SEQ ID NO: 32.

In some embodiments described herein, is a bispecific molecule comprising an antigen binding region comprising a light chain variable region (VL) having 100% identity to amino acid sequence SEQ ID NO: 32. In some embodiments, the bispecific molecule further comprises a heavy chain variable region (VH) having at least 80% identity to amino acid sequence SEQ ID NO: 31. In some embodiments, the bispecific molecule further comprises a heavy chain variable region (VH) having at least 90% identity to amino acid sequence SEQ ID NO: 31. In some embodiments, the bispecific molecule further comprises a heavy chain variable region (VH) having at least 95% identity to amino acid sequence SEQ ID NO: 31. In some embodiments, the bispecific molecule further comprises a heavy chain variable region (VH) having at least 99% identity to amino acid sequence SEQ ID NO: 31. In some embodiments, the bispecific molecule further comprises a heavy chain variable region (VH) having 100% identity to amino acid sequence SEQ ID NO: 31.

In some embodiments described herein, is a bispecific molecule comprising a heavy chain variable region (VH) having at least 80% identity to amino acid sequence SEQ ID NO: 31 and a light chain variable region (VL) having at least 80% identity to amino acid sequence SEQ ID NO: 32. In some embodiments, the bispecific molecule comprises the light chain variable region (VL) having at least 85% identity to amino acid sequence SEQ ID NO: 32. In some embodiments, the bispecific molecule comprises the light chain variable region (VL) having at least 90% identity to amino acid sequence SEQ ID NO: 32. In some embodiments, the bispecific molecule comprises the light chain variable region (VL) having at least 95% identity to amino acid sequence SEQ ID NO: 32. In some embodiments, the bispecific molecule comprises the light chain variable region (VL) having at least 99% identity to amino acid sequence SEQ ID NO: 32. In some embodiments, the bispecific molecule comprises the light chain variable region (VL) having 100% identity to amino acid sequence SEQ ID NO: 32. In some embodiments, the bispecific molecule comprises the heavy chain variable region (VH) having at least 85% identity to amino acid sequence SEQ ID NO: 31. In some embodiments, the bispecific molecule comprises the heavy chain variable region (VH) having at least 90% identity to amino acid sequence SEQ ID NO: 31. In some embodiments, the bispecific molecule comprises the heavy chain variable region (VH) having at least 95% identity to amino acid sequence SEQ ID NO: 31. In some embodiments, the bispecific molecule comprises the heavy chain variable region (VH) having at least 99% identity to amino acid sequence SEQ ID NO: 31. In some embodiments, the bispecific molecule comprises the heavy chain variable region (VH) having at least 100% identity to amino acid sequence SEQ ID NO: 31.

In some embodiments described herein, is a bispecific molecule comprising a heavy chain sequence comprising a complementarity determining region (CDR) H1 having at least 80% identity to amino acid sequence SEQ ID NO: 45, a CDR H2 having at least 80% identity to amino acid sequence SEQ ID NO: 46, and a CDR H3 having at least 80% identity to amino acid sequence SEQ ID NO: 47, and a light chain sequence comprising a CDR L1 having at least 80% identity to amino acid sequence SEQ ID NO: 48, a CDR L2 having at least 80% identity to amino acid sequence SEQ ID NO: 49, and a CDR L3 having at least 80% identity to amino acid sequence SEQ ID NO: 50. In some embodiments, the bispecific molecule comprises the light chain sequence comprising the CDR L1 having at least 85% identity to amino acid sequence SEQ ID NO: 48, the CDR L2 having at least 85% identity to amino acid sequence SEQ ID NO: 49, and the CDR L3 having at least 85% identity to amino acid sequence SEQ ID NO: 50. In some embodiments, the bispecific molecule comprises the light chain sequence comprising the CDR L1 having at least 90% identity to amino acid sequence SEQ ID NO: 48, the CDR L2 having at least 90% identity to amino acid sequence SEQ ID NO: 49, and the CDR L3 having at least 90% identity to amino acid sequence SEQ ID NO: 50. In some embodiments, the bispecific molecule comprises light chain sequence comprising the CDR L1 having at least 95% identity to amino acid sequence SEQ ID NO: 48, the CDR L2 having at least 95% identity to amino acid sequence SEQ ID NO: 49, and the CDR L3 having at least 95% identity to amino acid sequence SEQ ID NO: 50. In some embodiments, the bispecific molecule comprises the light chain sequence comprising the CDR L1 having at least 99% identity to amino acid sequence SEQ ID NO: 48, the CDR L2 having at least 99% identity to amino acid sequence SEQ ID NO: 49, and the CDR L3 having at least 99% identity to amino acid sequence SEQ ID NO: 50. In some embodiments, the bispecific molecule comprises the light chain sequence comprising the CDR L1 having 100% identity to amino acid sequence SEQ ID NO: 48, the CDR L2 having 100% identity to amino acid sequence SEQ ID NO: 49, and the CDR L3 having at least 100% identity to amino acid sequence SEQ ID NO: 50. In some embodiments, the bispecific molecule comprises the heavy chain sequence comprising the CDR H1 having at least 85% identity to amino acid sequence SEQ ID NO: 45, the CDR H2 having at least 85% identity to amino acid sequence SEQ ID NO: 46, and the CDR H3 having at least 85% identity to amino acid sequence SEQ ID NO: 47. In some embodiments, the bispecific molecule comprises the heavy chain sequence comprising a the CDR H1 having at least 90% identity to amino acid sequence SEQ ID NO: 45, the CDR H2 having at least 90% identity to amino acid sequence SEQ ID NO: 46, and the CDR H3 having at least 90% identity to amino acid sequence SEQ ID NO: 47. In some embodiments, the bispecific molecule comprises the heavy chain sequence comprising a the CDR H1 having at least 95% identity to amino acid sequence SEQ ID NO: 45, the CDR H2 having at least 95% identity to amino acid sequence SEQ ID NO: 46, and the CDR H3 having at least 95% identity to amino acid sequence SEQ ID NO: 47. In some embodiments, the bispecific molecule comprises the heavy chain sequence comprising a the CDR H1 having at least 99% identity to amino acid sequence SEQ ID NO: 45, the CDR H2 having at least 99% identity to amino acid sequence SEQ ID NO: 46, and the CDR H3 having at least 99% identity to amino acid sequence SEQ ID NO: 47. In some embodiments, the bispecific molecule comprises the heavy chain sequence comprising a the CDR H1 having at least 100% identity to amino acid sequence SEQ ID NO: 45, the CDR H2 having at least 100% identity to amino acid sequence SEQ ID NO: 46, and the CDR H3 having at least 100% identity to amino acid sequence SEQ ID NO: 47.

In some embodiments described herein, is a bispecific molecule that binds to CD163 comprising at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 48, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 49, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 50. In some embodiments, bispecific molecules that bind to CD163 comprise at least one of a light chain CDR1 having an amino acid sequence 100% identical to an amino acid sequence set forth as SEQ ID NO: 48, a light chain CDR2 having an amino acid sequence 100% identical to an amino acid sequence set forth as SEQ ID NO: 49, and a light chain CDR3 having an amino acid sequence 100% identical to an amino acid sequence set forth as SEQ ID NO: 50.

In some embodiments described herein, is a bispecific molecule that binds to CD163 comprising at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 45, a heavy chain CDR2 having an amino acid sequence at least about 75%, 80%, 81%, 82%, 83%, 84%, 85%, 86%, 87%, 88%, 89%0, 90%, 91%, 92%, 93%, 94%, 95%, 96%, 97%, 98%, or 99% identical to an amino acid sequence set forth as SEQ ID NO: 46, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 47. In some embodiments, bispecific molecules that bind to CD163 comprise at least one of a heavy chain CDR1 having an amino acid sequence 100% identical to an amino acid sequence set forth as SEQ ID NO: 45, a heavy chain CDR2 having an amino acid sequence 100% identical to an amino acid sequence set forth as SEQ ID NO: 46, and a heavy chain CDR3 having an amino acid sequence 100% identical to an amino acid sequence set forth as SEQ ID NO: 47.

In some embodiments described herein, is a bispecific molecule that binds to CD163 comprising at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 48, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 49, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 50, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 45, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 46, and a heavy chain CDR3 having an amino acid sequence at least about 75%, 80%, 81%, 82%, 83%, 84%, 85%, 86%, 87%, 88%, 89%0, 90%, 91%, 92%, 93%, 94%, 95%, 96%, 97%, 98%, or 99% identical to an amino acid sequence set forth as SEQ ID NO: 47. In some embodiments, bispecific molecules that bind to CD163 comprise at least one of a light chain CDR1 having an amino acid sequence 100% identical to an amino acid sequence set forth as SEQ ID NO: 48, a light chain CDR2 having an amino acid sequence 100% identical to an amino acid sequence set forth as SEQ ID NO: 49, a light chain CDR3 having an amino acid sequence 100% identical to an amino acid sequence set forth as SEQ ID NO: 50, a heavy chain CDR1 having an amino acid sequence 100% identical to an amino acid sequence set forth as SEQ ID NO: 45, a heavy chain CDR2 having an amino acid sequence 100% identical to an amino acid sequence set forth as SEQ ID NO: 46, and a heavy chain CDR3 having an amino acid sequence 100% identical to an amino acid sequence set forth as SEQ ID NO: 47.

In some embodiments described herein, is a bispecific molecule comprising a VL and VH chain that confers binding specificity to CD163 wherein amino acids in the framework can be varied. In some embodiments, amino acid variation involves introduction of conservative amino acid substitutions. In some embodiments, bispecific molecules that bind to CD163 comprise a light chain variable (VL) region and a heavy chain variable (VH) region, wherein the VL region comprises a light chain CDR1 (CDRL1) having an amino acid sequence 100% identical to an amino acid sequence set forth as SEQ ID NO: 48, a light chain CDR2 (CDRL2) having an amino acid sequence 100% identical to an amino acid sequence set forth as SEQ ID NO: 49, a light chain CDR3 (CDRL3) having an amino acid sequence 100% identical to an amino acid sequence set forth as SEQ ID NO: 50 wherein the amino acid sequence of the VL region outside of CDRL1, CDRL2, and CDRL3 has an overall sequence identity of at least 75%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 32, and wherein the VH region comprises a heavy chain CDR1 (CDRH1) having an amino acid sequence 100% identical to an amino acid sequence set forth as SEQ ID NO: 45, a heavy chain CDR2 (CDRH2) having an amino acid sequence 100% identical to an amino acid sequence set forth as SEQ ID NO: 46, a heavy chain CDR3 (CDRH3) having an amino acid sequence 100% identical to an amino acid sequence set forth as SEQ ID NO: 47 wherein the amino acid sequence of the VH region outside of CDRH1, CDRH2, and CDRH3 has an overall sequence identity of at least 75%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 31. In some embodiments, the amino acid sequence of the VL region outside of CDRL1, CDRL2, and CDRL3 has an overall sequence identity of at least 90%, identity to SEQ ID NO: 32 and the amino acid sequence of the VH region outside of CDRH1, CDRH2, and CDRH3 has an overall sequence identity of at least 90% identity to SEQ ID NO: 31. In some embodiments, the amino acid sequence of the VL region outside of CDRL1, CDRL2, and CDRL3 has an overall sequence identity of at least 95%, identity to SEQ ID NO: 32 and the amino acid sequence of the VH region outside of CDRH1, CDRH2, and CDRH3 has an overall sequence identity of at least 95% identity to SEQ ID NO: 31. In some embodiments, the amino acid sequence of the VL region outside of CDRL1, CDRL2, and CDRL3 has an overall sequence identity of at least 99%, identity to SEQ ID NO: 32 and the amino acid sequence of the VH region outside of CDRH1, CDRH2, and CDRH3 has an overall sequence identity of at least 99% identity to SEQ ID NO: 31.

In some embodiments, bispecific molecules that bind to CD163 comprise a light chain variable (VL) region and a heavy chain variable (VH) region, wherein the VL region comprises a light chain CDR1 (CDRL1) having an amino acid sequence 100% identical to an amino acid sequence set forth as SEQ ID NO: 48, a light chain CDR2 (CDRL2) having an amino acid sequence 100% identical to an amino acid sequence set forth as SEQ ID NO: 49, a light chain CDR3 (CDRL3) having an amino acid sequence 100% identical to an amino acid sequence set forth as SEQ ID NO: 50 wherein the amino acid sequence of the VL region outside of CDRL1, CDRL2, and CDRL3 has an overall sequence identity of at least 75%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 32 and wherein the V gene usage of the VL region is a kappa light chain, and wherein the VH region comprises a heavy chain CDR1 (CDRH1) having an amino acid sequence 100% identical to an amino acid sequence set forth as SEQ ID NO: 45, a heavy chain CDR2 (CDRH2) having an amino acid sequence 100% identical to an amino acid sequence set forth as SEQ ID NO: 46, a heavy chain CDR3 (CDRH3) having an amino acid sequence 100% identical to an amino acid sequence set forth as SEQ ID NO: 47 wherein the amino acid sequence of the VH region outside of CDRH1, CDRH2, and CDRH3 has an overall sequence identity of at least 75%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 31 and wherein the V gene usage of the VH region is IGHV gene. In some embodiments, the amino acid sequence of the VL region outside of CDRL1, CDRL2, and CDRL3 has an overall sequence identity of at least 90%, identity to SEQ ID NO: 32 and the amino acid sequence of the VH region outside of CDRH1, CDRH2, and CDRH3 has an overall sequence identity of at least 90% identity to SEQ ID NO: 31. In some embodiments, the amino acid sequence of the VL region outside of CDRL1, CDRL2, and CDRL3 has an overall sequence identity of at least 95%, identity to SEQ ID NO: 32 and the amino acid sequence of the VH region outside of CDRH1, CDRH2, and CDRH3 has an overall sequence identity of at least 95% identity to SEQ ID NO: 31. In some embodiments, the amino acid sequence of the VL region outside of CDRL1, CDRL2, and CDRL3 has an overall sequence identity of at least 99%, identity to SEQ ID NO: 32 and the amino acid sequence of the VH region outside of CDRH1, CDRH2, and CDRH3 has an overall sequence identity of at least 99% identity to SEQ ID NO: 31. In some embodiments, the V gene usage of the VL region is VK1.O12. In some embodiments, the V gene usage of the VH region is a IGHV3 gene. In some embodiments, the V gene usage of the VH region is IGHV3.30-3.

### Method of Treatment

In certain aspects the subject matter described herein relates to a method of treating or preventing cancer in a subject suffering with cancer. In some embodiments, the method comprises administering to the subject a bispecific molecule comprising at least two antigen binding regions, wherein each antigen binding region binds a different antigen on The First Cell (TFC) of a cancer. In some embodiments, the method comprises administering to the subject a pharmaceutical composition comprising any one of the bispecific molecules described herein.

In some embodiments, the first antigen is a marker of epithelial cell lineage wherein the first antigen is a marker of epithelial cell lineage. In some embodiments, the epithelial marker is any one of the markers in FIG. 2 the marker of epithelial cell lineage is any one of the markers in FIG. 2. In some embodiments, the marker of epithelial cell lineage is epithelial cellular adhesion molecule (EpCAM). In some embodiments, the second antigen is a marker of macrophage cell lineage. In some embodiments, the macrophage marker is any of the markers in FIG. 1. In some embodiments, the marker of macrophage cell lineage is CD163. In some embodiments, both antigens are macrophage cell lineage markers. In some embodiments the first antigen is CD117, CD34, CD123, or any combination thereof and the second antigen is CD163. In some embodiments, the bispecific antibody, functional equivalent thereof, antigen binding fragment thereof, a derivative thereof, or an antibody-like bispecific molecule binds to two different antigens selected from FIG. 1.

In some embodiments, the bispecific molecule is a bispecific antibody. In some embodiments, the bispecific antibody is conjugated to a drug. In some embodiments, the drug is a toxin. In some embodiments, the drug is a chemotherapy agent. In some embodiments, the bispecific molecule comprises bi-nanobodies, BiTE, tandAbs, DARTs, DART-Fc, DARPin, scFv, scFv-HAS-scFV, and DNL-Fab3. In some embodiments, the bispecific molecule is a bispecific chimeric antigen receptor (CAR). In some embodiments, the bispecific molecule is a split-CAR-T. In some embodiments, the bispecific molecule is a Co-LOCKR comprising a first polypeptide and a second polypeptide, wherein the first polypeptide and the second polypeptide each bind a different antigen on TFC of a cancer. In some embodiments, the bispecific CAR is a synNotch CAR. In some embodiments, the bispecific CAR binds to a Co-LOCKR comprising a first polypeptide, a second polypeptide, and a third polypeptide, wherein the first polypeptide and the second polypeptide each bind a different antigen on TFC of a cancer, wherein the third polypeptide binds to the bispecific CAR, and wherein the third polypeptide is operably linked to the first polypeptide or the second polypeptide.

In some embodiments, the invention a method comprising administering to the subject a pharmaceutical composition comprising any one of the bispecific molecules described herein, wherein the first antigen binding region binds to EpCAM and the second antigen binding region binds to CD163. Antigen binding regions specific for EpCAM are described herein, including but not limited to in the section titled “Antigen Binding Regions or Domains Specific for EpCAM.” Antigen binding regions specific for CD163 are described herein, including but not limited to in the section titled “Antigen Binding Regions or Domains Specific for CD163.” The method of treatment of the invention includes using any of the bispecific molecules described herein of the antigen binding regions specific for EpCAM in combination with any of the antigen binding regions specific for CD163.

In some embodiments, the cancer comprises a solid tumor. In some embodiments, the cancer is breast cancer, brain tumor, gastrointestinal including stomach and colorectal, pancreatic cancer, kidney cancer, liver cancer, lung cancer, thymic carcinoma, ovarian cancer, prostate cancer, or endometrial cancer. In some embodiments, the cancer is a liquid cancer. In some embodiments, the liquid cancer is leukemia, lymphoma, or myeloma. In some embodiments, the liquid cancer is a B-cell malignancy. In some embodiments, the B-cell malignancy is multiple myeloma. In some embodiments, the B-cell malignancy is B-cell lymphoma. In some embodiments, the B-cell malignancy is diffuse large B-cell lymphoma (DLBCL). In some embodiments, the B-cell malignancy is non-Hodgkin lymphomas (NHL). In some embodiments, the B-cell malignancy is chronic lymphocytic leukemia (CLL). In some embodiments, the liquid cancer is acute myeloid leukemia (AML). In some embodiments, the liquid cancer is a myeloid neoplasm. In some embodiments, the liquid cancer is myelodysplastic syndrome (MDS), myeloproliferative neoplasm (MPN), MDS/MPN overlap syndrome, acute myeloid leukemia or chronic myeloid leukemia. In some embodiments, the TFC is a metastatic TFC.

### Myeloid Leukemias

In some embodiments, the cancer is a myeloid leukemia. In some embodiments, the myeloid leukemia TFC expresses a first antigen and second antigen. In some embodiments, the first antigen and second antigen are both markers of macrophage cell lineage. In some embodiments, the first antigen and the second antigen are a pair of any one of the markers in FIG. 1. In some embodiments, the first antigen is CD117, CD34, CD123 or a combination thereof. In some embodiments, the second antigen is CD163. In some embodiments, the bispecific molecule disclosed here binds two antigens, both of which are markers of macrophage cell lineage.

### Myeloid Neoplasms

In some embodiments, the cancer is a myeloid neoplasm. In some embodiments, the myeloid neoplasm is a myelodysplastic syndrome (MDS). In some embodiments, the myeloid neoplasm is a myeloproliferative neoplasm (MPN). In some embodiments, the myeloid neoplasm is an overlap of MDS/MPN syndromes. In some embodiments, the myeloid neoplasm is an acute or chronic myeloid leukemia.

### Cancer Treatments

There are a number of cancer treatment options available today. Surgery is performed to remove the cancer or tumor mass. Chemotherapy includes administration of toxic drugs to patients to kill cancer cells. Radiation therapy utilizes high-powered energy beams, such as X-rays or protons, to kill cancer cells. Bone marrow can be transplanted from a healthy individual to a cancer patient, replacing the patient's own diseased bone marrow. The bone marrow produces blood cells from blood stem cells and the bone marrow transplant is performed in an effort to treat or cure liquid cancers. A bone marrow transplant also allows the use of higher doses of chemotherapy to treat cancer patients. Immunotherapy uses the body's own immune system to fight cancer. Immunotherapy can help the patient's immune system to recognize the cancerous cells and attack them. Hormone therapy can be used in the treatment of breast cancer or prostate cancer, which can be supported by the body's hormones. Blocking their effects on the body may stop the cancer from growing. Cryoablation kills cancer cells by lowering the temperature using a thin needle (cryoprobe) inserted through the patient's skin and directed into the cancerous tumor. Radiofrequency ablation uses electrical energy to heat cancer cells, thus killing them. High-frequency energy is directed through a needle, which causes the surrounding tissue to heat up. Each of these treatments can be administered in conjunction with the methods described herein.

### Method of Diagnosing

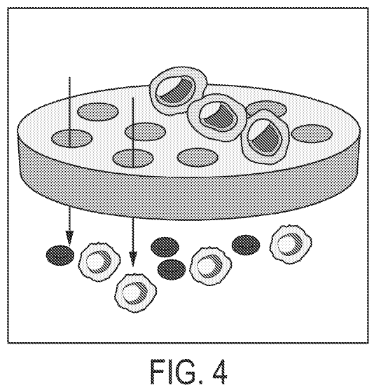

In certain aspects the subject matter described herein relates to a method of diagnosing cancer. In some embodiments, the method comprises detecting a cell expressing at least one marker of epithelial cell lineage and at least one marker of macrophage cell lineage. In some embodiments, the at least one marker of epithelial cell lineage is any one of the markers in FIG. 2. In some embodiments, the at least one marker of epithelial cell lineage is epithelial cellular adhesion molecule (EpCAM). In some embodiments, the at least one marker of macrophage cell lineage is any one of the markers in FIG. 1. In some embodiments, the at least one marker of macrophage cell lineage is CD163. In some embodiments, the method comprises detecting a cell expressing at least two macrophage cell lineage markers. In some embodiments the first marker is CD117, CD34, CD123, or any combination thereof and the second marker is CD163. In some embodiments, at least two markers of macrophage cell lineage is any two of the markers in FIG. 1. In some embodiments, the detecting comprises an assay wherein a bispecific molecule binds to the at least one marker of epithelial cell lineage and the at least one marker of macrophage cell lineage. In some embodiments, the detecting comprises an assay wherein a bispecific molecule binds to the at least two markers of macrophage cell lineage. In some embodiments, the detecting comprises a flow cytometry assay. In some embodiments, the detecting comprises an immunostaining assay. In some embodiments, antibodies that bind each antigen are each conjugated with a different fluorochrome and the fluorochromes are detected using either flow cytometry or immunostaining. In some embodiments, the immunostaining is an immunofluorescence staining. In some embodiments, the detecting comprises a step of separating cells by size, as shown in FIG. 4, prior to the assay. In some embodiments, large cells are selected for during size separation and assayed.

In some embodiments, the bispecific molecule is a bispecific antibody. In some embodiments, the bispecific antibody is conjugated to a drug. In some embodiments, the drug is a toxin. In some embodiments, the drug is a chemotherapy agent. In some embodiments, the bispecific molecule comprises bi-nanobodies, BiTE, tandAbs, DARTs, DART-Fc, DARPin, scFv, scFv-HAS-scFV, and DNL-Fab3. In some embodiments, the bispecific molecule is a bispecific chimeric antigen receptor (CAR). In some embodiments, the bispecific molecule is a Co-LOCKR comprising a first polypeptide and a second polypeptide, wherein the first polypeptide and the second polypeptide each bind a different antigen on TFC of a cancer. In some embodiments, the bispecific CAR is a synNotch CAR. In some embodiments, the bispecific CAR binds to a Co-LOCKR comprising a first polypeptide, a second polypeptide, and a third polypeptide, wherein the first polypeptide and the second polypeptide each bind a different antigen on TFC of a cancer, wherein the third polypeptide binds to the bispecific CAR, and wherein the third polypeptide is operably linked to the first polypeptide or the second polypeptide.

In some embodiments, the invention provides a method comprising detecting a cell expressing at least one marker of epithelial cell lineage and at least one marker of macrophage cell lineage using any one of the bispecific molecules described herein, wherein the first antigen binding region binds to EpCAM and the second antigen binding region binds to CD163. Antigen binding regions specific for EpCAM are described herein, including but not limited to in the section titled “Antigen Binding Regions or Domains Specific for EpCAM.” Antigen binding regions specific for CD163 are described herein, including but not limited to in the section titled “Antigen Binding Regions or Domains Specific for CD163.” The method of diagnosis of the invention includes using any of the bispecific molecules described herein of the antigen binding regions specific for EpCAM in combination with any of the antigen binding regions specific for CD163.

### Pharmaceutical Compositions

In certain aspects, also provided are pharmaceutical compositions comprising the above-described bispecific molecules. In some embodiments, the subject matter described herein relates to a pharmaceutical composition comprising an effective amount of the bispecific molecules described herein and a pharmaceutically-acceptable diluent, carrier or excipient.

As used herein, ‘pharmaceutical composition’ means a therapeutically effective formulation according to the invention. A ‘therapeutically effective amount’, or ‘effective amount’, or ‘therapeutically effective’, as used herein, refers to that amount which provides a therapeutic effect for a given condition and administration regimen. A therapeutically effective amount can be determined by a skilled person based on patient characteristics, such as age, weight, sex, condition, complications, other diseases, etc., as is well known in the art.

In some embodiments, the pharmaceutical compositions described herein can be administered as solid compositions. In some embodiments, the solid compositions comprise excipients including but not limited to lactose, starch, cellulose, milk sugar or high molecular weight polyethylene glycols. In some embodiments, the pharmaceutical compositions described herein can be administered as aqueous suspensions and/or elixirs. In some embodiments, the pharmaceutical compositions described herein may be combined with various sweetening or flavouring agents, coloring matter or dyes, with emulsifying and/or suspending agents and with diluents such as water, ethanol, propylene glycol and glycerin, and combinations thereof.

In some embodiments, the pharmaceutical compositions described herein can be administered parenterally, for example, intravenously, intra-arterially, intraperitoneally, intra-thecally, intraventricularly, intrasternally, intracranially, intra-muscularly or subcutaneously, or they may be administered by infusion techniques. In some embodiments, the pharmaceutical compositions described herein can be administered in the form of a sterile aqueous solution which may contain other substances, for example, enough salts or glucose to make the solution isotonic with blood. The preparation of suitable parenteral formulations under sterile conditions is readily accomplished by standard pharmaceutical techniques well-known to those skilled in the art.

In some embodiments, pharmaceutical compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The pharmaceutical compositions can be presented in unit-dose or multi-dose containers. The pharmaceutical compositions can be sealed ampoules or vials. The pharmaceutical compositions can be stored in a freeze-dried (lyophilised) condition requiring only the addition of the sterile liquid carrier, such as water for injections, immediately prior to use.

### Polynucleotides

In certain aspects, also provided are polynucleotides encoding the bispecific molecules described herein or portions thereof. In some embodiments, polynucleotides that encode the bispecific molecules described herein or portions thereof are isolated from cells expressing the bispecific molecules described herein or portions thereof, according to methods available in the art, including amplification by polymerase chain reaction. One or more polynucleotides encoding one or more bispecific molecules described herein or portions thereof can be subcloned into one or more expression vectors. In some embodiments, the expression vector comprising a polynucleotide encoding the bispecific molecule or portion thereof can be used to recombinantly produce the bispecific molecule or portion thereof described herein, using procedures known in the art.

### Vectors

In certain aspects, also provided are vectors comprising one or more polynucleotide encoding one or more bispecific molecule or portion thereof as described herein. In some embodiments, the bispecific molecules or portions thereof as described here are produced recombinantly, using any suitable vectors and methods known in the art. Suitable expression vectors include but are not limited to a pcDNA3.4, a pcDNA3.3 Topo, pOptiVec, pSG5L, pDEST27, pCI, pIRES, pBApo, pSF-CMV and pEF4/V5 His A.

### Viruses

In certain aspects, also provided are viruses comprising a polynucleotide encoding the bispecific molecules. Suitable virus-based protein expression systems are known in the art and include but are not limited to a lentivirus expression system and an adenovirus expression system. Suitable lentivirus vectors include but are not limited to pHIV-dTomato, pAWp11, LRG2.1, LT3, LentiV, pCCL, pCS, and pHIV-eGFP. Suitable adenoviral vectors include but are not limited to pAd/CMV/V5-DEST pAdenoX, pICPIS, pAAV, and pAd/PL-DEST. Methods of protein expression using virus-based expression systems are well known in the art.

### Cells

In certain aspects, also provided are genetically engineered cells, transformed or transduced host cells, comprising the bispecific molecules and/or polynucleotides encoding the bispecific molecules. Suitable cells known in the art include but are not limited to T cells, HL-60 cells, CHO cells, HEK 293 cells, 293 T cells, E. coli, DH5a. These cells can be genetically engineered, transformed or transduced with the bispecific molecules and/or polynucleotides encoding the bispecific molecules by any method known in the art.

### REFERENCES

1 Borot, F., Mukherjee, S. & Ali, A. M. Challenges and Solutions to Bringing Chimeric Antigen Receptor T-Cell Therapy to Myeloid Malignancies. Cancer J 27, 143-150, doi:10.1097/PPO.0000000000000512 (2021).
2 Borot, F. et al. Gene-edited stem cells enable CD33-directed immune therapy for myeloid malignancies. Proc Natl Acad Sci USA 116, 11978-11987, doi:10.1073/pnas.1819992116 (2019).
3 Wang, H. F. et al. Cell fusion in cancer hallmarks: Current research status and future indications. Oncol Lett 22, 530, doi:10.3892/ol.2021.12791 (2021).
4 Vignery, A. Macrophage fusion: are somatic and cancer cells possible partners? Trends Cell Biol 15, 188-193, doi:10.1016/j.tcb.2005.02.008 (2005).
5 Sutton, T. L., Walker, B. S. & Wong, M. H. Circulating Hybrid Cells Join the Fray of Circulating Cellular Biomarkers. Cell Mol Gastroenterol Hepatol 8, 595-607, doi:10.1016/j.jcmgh.2019.07.002 (2019).
6 Shabo, I. et al. Macrophage traits in cancer cells are induced by macrophage-cancer cell fusion and cannot be explained by cellular interaction. BMC Cancer 15, 922, doi:10.1186/s12885-015-1935-0 (2015).
7 Adams, D. L. et al. Circulating giant macrophages as a potential biomarker of solid tumors. Proc Natl Acad Sci USA 111, 3514-3519, doi:10.1073/pnas.1320198111 (2014).
8 Shabo, I., Olsson, H., Sun, X. F. & Svanvik, J. Expression of the macrophage antigen CD163 in rectal cancer cells is associated with early local recurrence and reduced survival time. Int J Cancer 125, 1826-1831, doi:10.1002/ijc.24506 (2009).
9 Aljabery, F., Olsson, H., Gimm, O., Jahnson, S. & Shabo, I. M2-macrophage infiltration and macrophage traits of tumor cells in urinary bladder cancer. Urol Oncol 36, 159 e119-159 e126, doi:10.1016/j.urolonc.2017.11.020 (2018).
10 Sodi, S. A. et al. Melanoma×macrophage fusion hybrids acquire increased melanogenesis and metastatic potential: altered N-glycosylation as an underlying mechanism. Pigment Cell Res 11, 299-309, doi:10.1111/j.1600-0749.1998.tb00739.x (1998).
11 Rachkovsky, M. et al. Melanoma×macrophage hybrids with enhanced metastatic potential. Clin Exp Metastasis 16, 299-312, doi:10.1023/a:1006557228604 (1998).
12 Dittmar, T. et al. Recurrence cancer stem cells—made by cell fusion? Med Hypotheses 73, 542-547, doi:10.1016/j.mehy.2009.05.044 (2009).
13 Clawson, G. A. et al. “Stealth dissemination” of macrophage-tumor cell fusions cultured from blood of patients with pancreatic ductal adenocarcinoma. PLoS One 12, e0184451, doi:10.1371/journal.pone.0184451 (2017).
14 Aguirre, L. A. et al. Tumor stem cells fuse with monocytes to form highly invasive tumor-hybrid cells. Oncoimmunology 9, 1773204, doi:10.1080/2162402X.2020.1773204 (2020).
15 Lajoie, M. J. et al. Designed protein logic to target cells with precise combinations of surface antigens. Science 369, 1637-1643, doi:10.1126/science.aba6527 (2020).
16 Roybal, K. T. et al. Engineering T Cells with Customized Therapeutic Response Programs Using Synthetic Notch Receptors. Cell 167, 419-432 e416, doi:10.1016/j.cell.2016.09.011 (2016).
17 Morsut, L. et al. Engineering Customized Cell Sensing and Response Behaviors Using Synthetic Notch Receptors. Cell 164, 780-791, doi:10.1016/j.cell.2016.01.012 (2016).
18 Kloss, C. C., Condomines, M., Cartellieri, M., Bachmann, M. & Sadelain, M. Combinatorial antigen recognition with balanced signaling promotes selective tumor eradication by engineered T cells. Nat Biotechnol 31, 71-75, doi:10.1038/nbt.2459 (2013).
19 Lee, S. & Wong, W. W. The Most Logical Approach to Improve CAR T Cell Therapy. Cell Syst 11, 421-423, doi:10.1016/j.cels.2020.10.008 (2020).
20 Cho, J. H., Collins, J. J. & Wong, W. W. Universal Chimeric Antigen Receptors for Multiplexed and Logical Control of T Cell Responses. Cell 173, 1426-1438 e1411, doi:10.1016/j.cell.2018.03.038 (2018).
21 Kittel-Boselli, E. et al. Targeting Acute Myeloid Leukemia Using the RevCAR Platform: A Programmable, Switchable and Combinatorial Strategy. Cancers (Basel) 13, doi:10.3390/cancers13194785 (2021).
22 Feldmann, A. et al. Versatile chimeric antigen receptor platform for controllable and combinatorial T cell therapy. Oncoimmunology 9, 1785608, doi:10.1080/2162402X.2020.1785608 (2020).
23 Salzer, B. et al. Engineering AvidCARs for combinatorial antigen recognition and reversible control of CAR function. Nat Commun 11, 4166, doi:10.1038/s41467-020-17970-3 (2020).

### Example 1—Generation of a Cell Line Model for the Expression of Lineage Specific Antigens

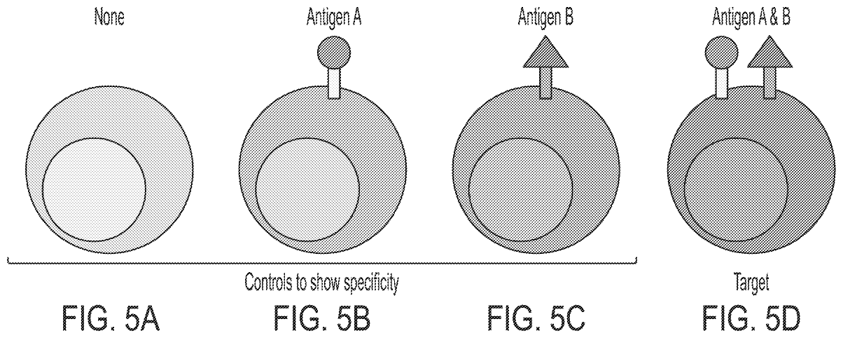

A cell line model was generated that recapitulates the expression of two lineage specific antigens (LSA) on The First Cell also referred to herein as the tumor macrophage hybrid (TMH) target cells (FIGS. 5A-D).

LSA selection: A TMH cell is formed by fusion of cells from two different lineages. The fusion results in reorganization of biomolecules including chromatin, transcriptome and proteome resulting in hybrid cells that retain expression of certain antigens from both the lineages (1-3). Theoretically, LSA from either lineage should be present on the hybrid cells. FIG. 1 and FIG. 2 show a list of CD antigens with epithelial or macrophage lineage. Expression of several LSAs on TMH are reported (4, 5). The LSA selected were EpCAM and CD163 based on the expression of these antigens on TMH (1, 2, 4, 6-19). Protein sequences of EpCAM or CD163 and their isoforms can be found in Table 1 below.

Cell line: HL-60 (CCL-240; ATCC) cell line is used to express the LSA. HL-60 cells were cultured in IMDM media supplemented with 10% fetal bovine serum (FBS) and 1× penicillin and streptomycin at 37 C and 5% CO₂ in a humidified chamber.

Plasmid DNA: EpCAM (HsCD00954335; DNASU Repository) and CD163 (HsCD00861066; DNASU Repository) encoding plasmids with lentiviral backbone (pLenti6.3/V5-DEST) and V5 tag at C-terminus were amplified in E. coli (DH5a) cells and plasmid DNA was purified using Qiagen Maxi kit. Sequences of EpCAM or CD163 expressed in the HL-60 cells are listed in Table 2.

Lentivirus: Lentiviral particles of EpCAM and CD163 expressing plasmids were obtained by co-transfecting with helper plasmids (encoding VSVG coat protein) in 293 cells. 239 cells were cultured in DMEM media supplemented with 10% fetal bovine serum (FBS) and 1× penicillin and streptomycin at 37° C. and 5% CO₂ in a humidified chamber. Lentiviral particles from the cell supernatant were purified using PEG and resuspended in IMDM media.

Viral transduction and selection: Lentiviral particles were used to transduce HL-60 cells. Concentrated lentiviral particles were co-incubated with fibronectin treated cell culture, and with HL-60 cells. After 48 h of co-incubation, cells expressing EpCAM or CD163 or both were selected using the antibiotic blasticidin.

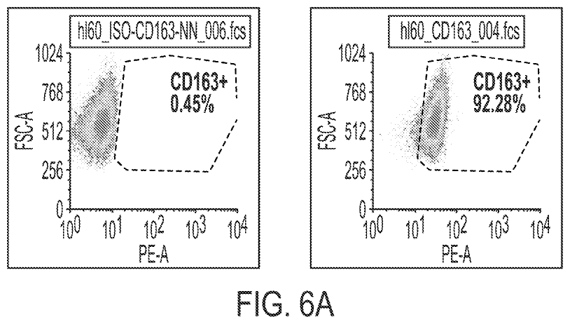

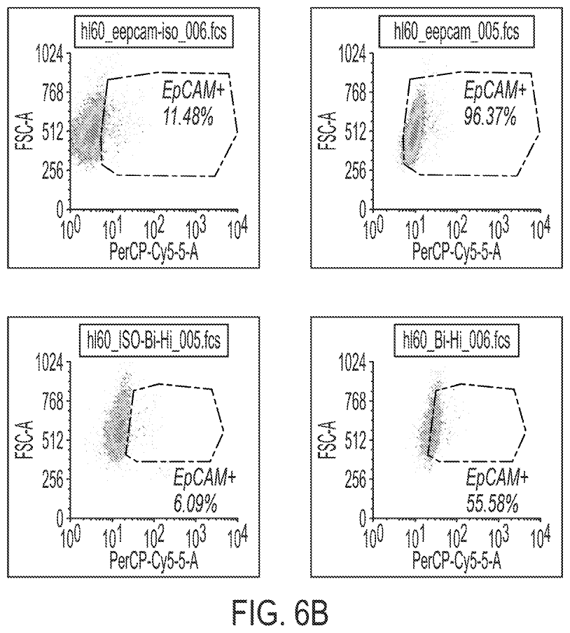

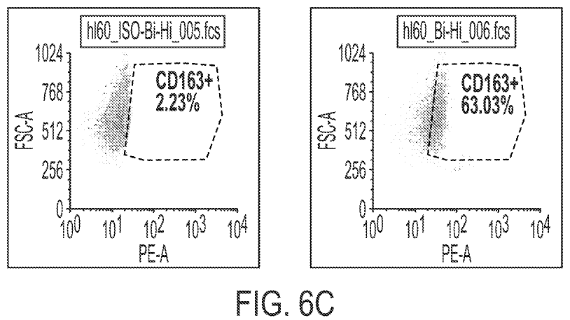

Phenotyping: The expression of EpCAM and CD163 was measured using flow cytometry (FIGS. 6A-C). Cells were stained with 1:100 dilution of fluorochrome-conjugate monoclonal antibodies recognizing human EpCAM (Clone 9C4 conjugated to PerCP/Cyanine5.5; Biolegend 324213) or CD163 (Clone RM3/1 conjugated to PE; Biolegend 326506) and data acquired on BD LSRII flow cytometer and Diva Software. Flow cytometry data was analyzed using either FlowJo (FlowJo LLC) or FCS Express (De Novo Software).

TABLE 1
Sequences of EpCAM and CD163 with uniprot IDs.

EpCAM protein sequence (Uniprot ID P16422):
MAPPQVLAFGLLLAAATATFAAAQEECVCENYKLAVNCFVNNNRQCQCTSVGAQNTVICSK
LAAKCLVMKAEMNGSKLGRRAKPEGALQNNDGLYDPDCDESGLFKAKQCNGTSMCWCVNTA
GVRRTDKDTEITCSERVRTYWIIIELKHKAREKPYDSKSLRTALQKEITTRYQLDPKFITS
ILYENNVITIDLVQNSSQKTQNDVDIADVAYYFEKDVKGESLFHSKKMDLTVNGEQLDLDP
GQTLIYYVDEKAPEFSMQGLKAGVIAVIVVVVIAVVAGIVVLVISRKKRMAKYEKAEIKEM
GEMHRELNA (SEQ ID NO. 7)
CD163 protein sequence (Uniprot ID Q86VB7-1):
MSKLRMVLLEDSGSADFRRHFVNLSPFTITVVLLLSACFVTSSLGGTDKELRLVDGENKCS
GRVEVKVQEEWGTVCNNGWSMEAVSVICNQLGCPTAIKAPGWANSSAGSGRIWMDHVSCRG
NESALWDCKHDGWGKHSNCTHQQDAGVTCSDGSNLEMRLTRGGNMCSGRIEIKFQGRWGTV
CDDNFNIDHASVICRQLECGSAVSFSGSSNFGEGSGPIWFDDLICNGNESALWNCKHQGWG
KHNCDHAEDAGVICSKGADLSLRLVDGVTECSGRLEVRFQGEWGTICDDGWDSYDAAVACK
QLGCPTAVTAIGRVNASKGFGHIWLDSVSCQGHEPAIWQCKHHEWGKHYCNHNEDAGVTCS
DGSDLELRLRGGGSRCAGTVEVEIQRLLGKVCDRGWGLKEADVVCRQLGCGSALKTSYQVY
SKIQATNTWLFLSSCNGNETSLWDCKNWQWGGLTCDHYEEAKITCSAHREPRLVGGDIPCS
GRVEVKHGDTWGSICDSDFSLEAASVLCRELQCGTVVSILGGAHFGEGNGQIWAEEFQCEG
HESHLSLCPVAPRPEGTCSHSRDVGVVCSRYTEIRLVNGKTPCEGRVELKTLGAWGSLCNS
HWDIEDAHVLCQQLKCGVALSTPGGARFGKGNGQIWRHMFHCTGTEQHMGDCPVTALGASL
CPSEQVASVICSGNQSQTLSSCNSSSLGPTRPTIPEESAVACIESGQLRLVNGGGRCAGRV
EIYHEGSWGTICDDSWDLSDAHVVCRQLGCGEAINATGSAHFGEGTGPIWLDEMKCNGKES
RIWQCHSHGWGQQNCRHKEDAGVICSEFMSLRLTSEASREACAGRLEVFYNGAWGTVGKSS
MSETTVGVVCRQLGCADKGKINPASLDKAMSIPMWVDNVQCPKGPDTLWQCPSSPWEKRLA
SPSEETWITCDNKIRLQEGPTSCSGRVEIWHGGSWGTVCDDSWDLDDAQVVCQQLGCGPAL
KAFKEAEFGQGTGPIWLNEVKCKGNESSLWDCPARRWGHSECGHKEDAAVNCTDISVQKTP
QKATTGRSSRQSSFIAVGILGVVLLAIFVALFFLTKKRRQRQRLAVSSRGENLVHQIQYRE
MNSCLNADDLDLMNSSENSHESADFSAAELISVSKFLPISGMEKEAILSHTEKENGNL
(SEQ ID NO. 8)
CD163 long tail variant (Uniprot ID Q86VB7-2):
MSKLRMVLLEDSGSADFRRHFVNLSPFTITVVLLLSACFVTSSLGGTDKELRLVDGENKCS
GRVEVKVQEEWGTVCNNGWSMEAVSVICNQLGCPTAIKAPGWANSSAGSGRIWMDHVSCRG
NESALWDCKHDGWGKHSNCTHQQDAGVTCSDGSNLEMRLTRGGNMCSGRIEIKFQGRWGTV
CDDNFNIDHASVICRQLECGSAVSFSGSSNFGEGSGPIWFDDLICNGNESALWNCKHQGWG
KHNCDHAEDAGVICSKGADLSLRLVDGVTECSGRLEVRFQGEWGTICDDGWDSYDAAVACK
QLGCPTAVTAIGRVNASKGFGHIWLDSVSCQGHEPAIWQCKHHEWGKHYCNHNEDAGVTCS
DGSDLELRLRGGGSRCAGTVEVEIQRLLGKVCDRGWGLKEADVVCRQLGCGSALKTSYQVY
SKIQATNTWLFLSSCNGNETSLWDCKNWQWGGLTCDHYEEAKITCSAHREPRLVGGDIPCS
GRVEVKHGDTWGSICDSDFSLEAASVLCRELQCGTVVSILGGAHFGEGNGQIWAEEFQCEG
HESHLSLCPVAPRPEGTCSHSRDVGVVCSRYTEIRLVNGKTPCEGRVELKTLGAWGSLCNS
HWDIEDAHVLCQQLKCGVALSTPGGARFGKGNGQIWRHMFHCTGTEQHMGDCPVTALGASL
CPSEQVASVICSGNQSQTLSSCNSSSLGPTRPTIPEESAVACIESGQLRLVNGGGRCAGRV
EIYHEGSWGTICDDSWDLSDAHVVCRQLGCGEAINATGSAHFGEGTGPIWLDEMKCNGKES
RIWQCHSHGWGQQNCRHKEDAGVICSEFMSLRLTSEASREACAGRLEVFYNGAWGTVGKSS
MSETTVGVVCRQLGCADKGKINPASLDKAMSIPMWVDNVQCPKGPDTLWQCPSSPWEKRLA
SPSEETWITCDNKIRLQEGPTSCSGRVEIWHGGSWGTVCDDSWDLDDAQVVCQQLGCGPAL
KAFKEAEFGQGTGPIWLNEVKCKGNESSLWDCPARRWGHSECGHKEDAAVNCTDISVQKTP
QKATTGRSSRQSSFIAVGILGVVLLAIFVALFFLTKKRRQRQRLAVSSRGENLVHQIQYRE
MNSCLNADDLDLMNSSGLWVLGGSIAQGFRSVAAVEAQTFYFDKQLKKSKNVIGSLDAYNG
QE (SEQ ID NO. 9)
CD163 short tail variant (Uniprot ID Q86VB7-3):
MSKLRMVLLEDSGSADFRRHFVNLSPFTITVVLLLSACFVTSSLGGTDKELRLVDGENKCS
GRVEVKVQEEWGTVCNNGWSMEAVSVICNQLGCPTAIKAPGWANSSAGSGRIWMDHVSCRG
NESALWDCKHDGWGKHSNCTHQQDAGVTCSDGSNLEMRLTRGGNMCSGRIEIKFQGRWGTV
CDDNFNIDHASVICRQLECGSAVSFSGSSNFGEGSGPIWFDDLICNGNESALWNCKHQGWG
KHNCDHAEDAGVICSKGADLSLRLVDGVTECSGRLEVRFQGEWGTICDDGWDSYDAAVACK
QLGCPTAVTAIGRVNASKGFGHIWLDSVSCQGHEPAIWQCKHHEWGKHYCNHNEDAGVTCS
DGSDLELRLRGGGSRCAGTVEVEIQRLLGKVCDRGWGLKEADVVCRQLGCGSALKTSYQVY
SKIQATNTWLFLSSCNGNETSLWDCKNWQWGGLTCDHYEEAKITCSAHREPRLVGGDIPCS
GRVEVKHGDTWGSICDSDFSLEAASVLCRELQCGTVVSILGGAHFGEGNGQIWAEEFQCEG
HESHLSLCPVAPRPEGTCSHSRDVGVVCSRYTEIRLVNGKTPCEGRVELKTLGAWGSLCNS
HWDIEDAHVLCQQLKCGVALSTPGGARFGKGNGQIWRHMFHCTGTEQHMGDCPVTALGASL
CPSEQVASVICSGNQSQTLSSCNSSSLGPTRPTIPEESAVACIESGQLRLVNGGGRCAGRV
EIYHEGSWGTICDDSWDLSDAHVVCRQLGCGEAINATGSAHFGEGTGPIWLDEMKCNGKES
RIWQCHSHGWGQQNCRHKEDAGVICSEFMSLRLTSEASREACAGRLEVFYNGAWGTVGKSS
MSETTVGVVCRQLGCADKGKINPASLDKAMSIPMWVDNVQCPKGPDTLWQCPSSPWEKRLA
SPSEETWITCDNKIRLQEGPTSCSGRVEIWHGGSWGTVCDDSWDLDDAQVVCQQLGCGPAL
KAFKEAEFGQGTGPIWLNEVKCKGNESSLWDCPARRWGHSECGHKEDAAVNCTDISVQKTP
QKATTGRSSRQSSFIAVGILGVVLLAIFVALFFLTKKRRQRQRLAVSSRGENLVHQIQYRE
MNSCLNADDLDLMNSSGGHSEPH (SEQ ID NO. 10)
CD163 variant (Uniprot ID Q86VB7-4):
MSKLRMVLLEDSGSADFRRHFVNLSPFTITVVLLLSACFVTSSLGGTDKELRLVDGENKCS
GRVEVKVQEEWGTVCNNGWSMEAVSVICNQLGCPTAIKAPGWANSSAGSGRIWMDHVSCRG
NESALWDCKHDGWGKHSNCTHQQDAGVTCSDGSNLEMRLTRGGNMCSGRIEIKFQGRWGTV
CDDNFNIDHASVICRQLECGSAVSFSGSSNFGEGSGPIWFDDLICNGNESALWNCKHQGWG
KHNCDHAEDAGVICSKGADLSLRLVDGVTECSGRLEVRFQGEWGTICDDGWDSYDAAVACK
QLGCPTAVTAIGRVNASKGFGHIWLDSVSCQGHEPAIWQCKHHEWGKHYCNHNEDAGVTCS
DGSDLELRLRGGGSRCAGTVEVEIQRLLGKVCDRGWGLKEADVVCRQLGCGSALKTSYQVY
SKIQATNTWLFLSSCNGNETSLWDCKNWQWGGLTCDHYEEAKITCSAHREPRLVGGDIPCS
GRVEVKHGDTWGSICDSDFSLEAASVLCRELQCGTVVSILGGAHFGEGNGQIWAEEFQCEG
HESHLSLCPVAPRPEGTCSHSRDVGVVCSSKTQKTSLIGSYTVKGTGLGSHSCLFLKPCLL
PGYTEIRLVNGKTPCEGRVELKTLGAWGSLCNSHWDIEDAHVLCQQLKCGVALSTPGGARF
GKGNGQIWRHMFHCTGTEQHMGDCPVTALGASLCPSEQVASVICSGNQSQTLSSCNSSSLG
PTRPTIPEESAVACIESGQLRLVNGGGRCAGRVEIYHEGSWGTICDDSWDLSDAHVVCRQL
GCGEAINATGSAHFGEGTGPIWLDEMKCNGKESRIWQCHSHGWGQQNCRHKEDAGVICSEF
MSLRLTSEASREACAGRLEVFYNGAWGTVGKSSMSETTVGVVCRQLGCADKGKINPASLDK
AMSIPMWVDNVQCPKGPDTLWQCPSSPWEKRLASPSEETWITCDNKIRLQEGPTSCSGRVE
IWHGGSWGTVCDDSWDLDDAQVVCQQLGCGPALKAFKEAEFGQGTGPIWLNEVKCKGNESS
LWDCPARRWGHSECGHKEDAAVNCTDISVQKTPQKATTGRSSRQSSFIAVGILGVVLLAIF
VALFFLTKKRRQRQRLAVSSRGENLVHQIQYREMNSCLNADDLDLMNSSGGHSEPH
(SEQ ID NO. 11)

TABLE 2
Protein sequences expressed in the HL-60 cells.

EpCAM protein sequence (Uniprot ID P16422) with V5 tag (underlined) at C-terminus:
MAPPQVLAFGLLLAAATATFAAAQEECVCENYKLAVNCFVNNNRQCQCTSVGAQNTVICSK
LAAKCLVMKAEMNGSKLGRRAKPEGALQNNDGLYDPDCDESGLFKAKQCNGTSMCWCVNTA
GVRRTDKDTEITCSERVRTYWIIIELKHKAREKPYDSKSLRTALQKEITTRYQLDPKFITS
ILYENNVITIDLVQNSSQKTQNDVDIADVAYYFEKDVKGESLFHSKKMDLTVNGEQLDLDP
GQTLIYYVDEKAPEFSMQGLKAGVIAVIVVVVIAVVAGIVVLVISRKKRMAKYEKAEIKEM
GEMHRELNADPAFLYKVVDIQHSGGRSSLEGPRFEGKPIPNPLLGLDSTRTG (SEQ ID
NO. 12)
CD163 protein sequence (Uniprot ID Q86VB7-1) with V5 tag (underlined) at C-terminus:
MSKLRMVLLEDSGSADFRRHFVNLSPFTITVVLLLSACFVTSSLGGTDKELRLVDGENKCS
GRVEVKVQEEWGTVCNNGWSMEAVSVICNQLGCPTAIKAPGWANSSAGSGRIWMDHVSCRG
NESALWDCKHDGWGKHSNCTHQQDAGVTCSDGSNLEMRLTRGGNMCSGRIEIKFQGRWGTV
CDDNFNIDHASVICRQLECGSAVSFSGSSNFGEGSGPIWFDDLICNGNESALWNCKHQGWG
KHNCDHAEDAGVICSKGADLSLRLVDGVTECSGRLEVRFQGEWGTICDDGWDSYDAAVACK
QLGCPTAVTAIGRVNASKGFGHIWLDSVSCQGHEPAIWQCKHHEWGKHYCNHNEDAGVTCS
DGSDLELRLRGGGSRCAGTVEVEIQRLLGKVCDRGWGLKEADVVCRQLGCGSALKTSYQVY
SKIQATNTWLFLSSCNGNETSLWDCKNWQWGGLTCDHYEEAKITCSAHREPRLVGGDIPCS
GRVEVKHGDTWGSICDSDFSLEAASVLCRELQCGTVVSILGGAHFGEGNGQIWAEEFQCEG
HESHLSLCPVAPRPEGTCSHSRDVGVVCSRYTEIRLVNGKTPCEGRVELKTLGAWGSLCNS
HWDIEDAHVLCQQLKCGVALSTPGGARFGKGNGQIWRHMFHCTGTEQHMGDCPVTALGASL
CPSEQVASVICSGNQSQTLSSCNSSSLGPTRPTIPEESAVACIESGQLRLVNGGGRCAGRV
EIYHEGSWGTICDDSWDLSDAHVVCRQLGCGEAINATGSAHFGEGTGPIWLDEMKCNGKES
RIWQCHSHGWGQQNCRHKEDAGVICSEFMSLRLTSEASREACAGRLEVFYNGAWGTVGKSS
MSETTVGVVCRQLGCADKGKINPASLDKAMSIPMWVDNVQCPKGPDTLWQCPSSPWEKRLA
SPSEETWITCDNKIRLQEGPTSCSGRVEIWHGGSWGTVCDDSWDLDDAQVVCQQLGCGPAL
KAFKEAEFGQGTGPIWLNEVKCKGNESSLWDCPARRWGHSECGHKEDAAVNCTDISVQKTP
QKATTGRSSRQSSFIAVGILGVVLLAIFVALFFLTKKRRQRQRLAVSSRGENLVHQIQYRE
MNSCLNADDLDLMNSSENSHESADFSAAELISVSKFLPISGMEKEAILSHTEKENGNLLNP
AFLYKVVDIQHSGGRSSLEGPRFEGKPIPNPLLGLDSTRTG (SEQ ID NO. 13)

### Example 2—Generation of Co-LOCKR Proteins

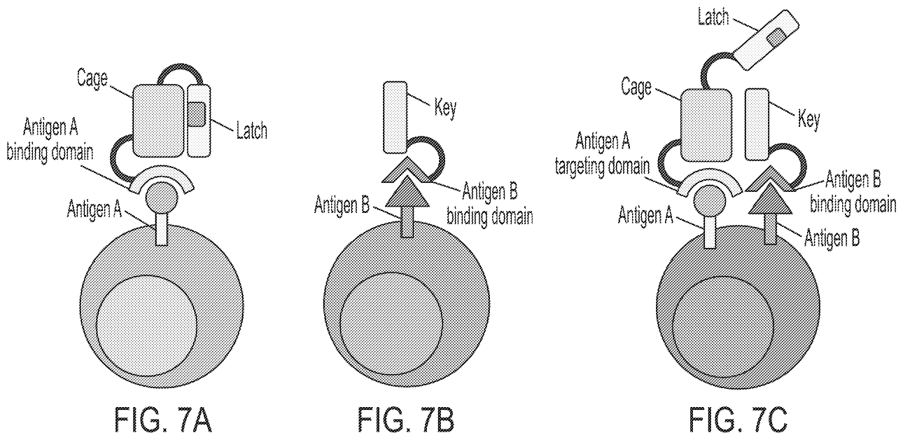

To achieve precision targeting of TMH which are characterized by the expression of LSA from two different lineages and to spare the cells from parental linage (expressing single LSA), co-localization-dependent protein-based logic-gated design was used that performs AND Boolean logic operations to target TMH with precise combinations of surface antigens (EpCAM or CD163) (20). De novo protein switches that activate when co-localized to compute AND logic have been described that include latching orthogonal cage-key proteins (Co-LOCKR). The Co-LOCKR consist of Cage and the Key proteins activating through a conformational change when the Cage and Key co-localize (FIG. 7A-C) (20). The CAGE module consists of “latch” that sequester a function peptide (Bim) in an inactive conformation but can bind an effector (Bcl2) upon conformation change. An effector can be a protein drug conjugate (Bcl2 conjugated to a cytotoxic compound) or a chimeric antigen receptor T-cells (CAR-T) expressing a Bcl2 CAR (20). Both Cage and Key are modular and can be attached to antigen binding domains to recruit the Cage and Key to cells expressing the target antigens (EpCAM or CD163). The antigen binding domains can be single chain variable fragments (ScFv) or Darpins or any molecule that has an affinity for the target.

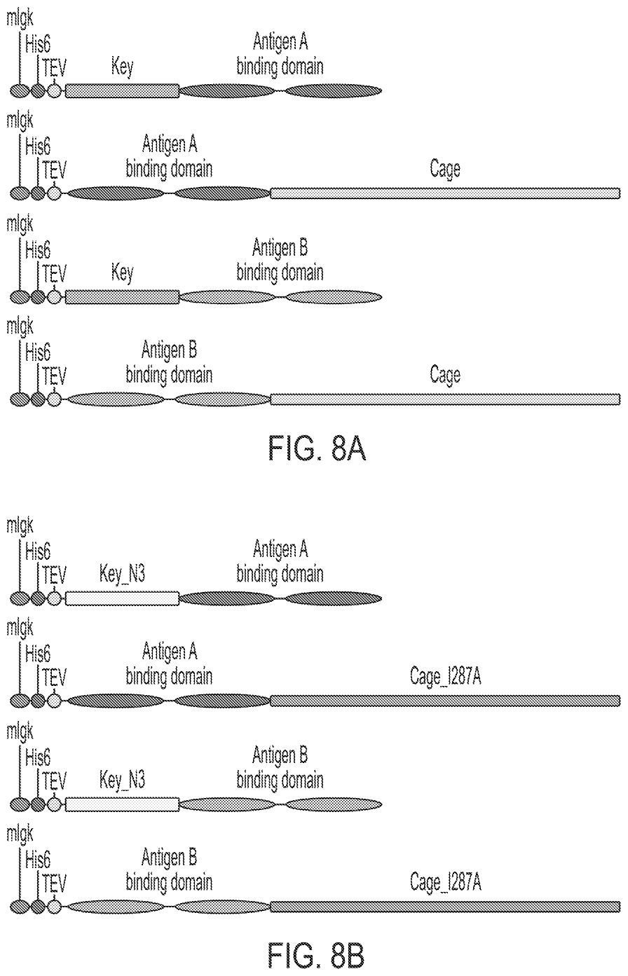

Design: Schematic design of EpCAM and CD163 targeting Cage and Key is described in FIGS. 8A-B. A typical Cage and Key targeting proteins contains a signal peptide from mouse immunoglobulin kappa (mIgk), a 6× histidine tag (His6) for protein purification, a tobacco etched virus (TEV) protease site to remove the tag, a key or Cage domain, and an antigen binding domain. Cage and Key proteins were designed for both EpCAM and CD163 using both the “wildtype” sequence or variants of Key and Cage resulting in a total 8 proteins (Table 3). For the antigen binding domain binding to EpCAM, we selected anti-EpCAM ScFv: the variable light chain (VL) and variable heavy chain (VH) sequences of anti-EpCAM ScFv have been described in U.S. Pat. No. 7,632,925 B2, the contents of which is incorporated herein by reference in its entirety. For the antigen binding domain binding to CD163, we selected anti-CD163 ScFv: the variable light chain (VL) and heavy chain (VH) sequences of anti-CD163 ScFv has been described in U.S. Pat. No. 9,724,426 B2, the contents of which is incorporated herein by reference in its entirety. For the antigen binding domain binding to CD163, the variable light chain (VL) and variable heavy chain (VH) sequences of anti-CD163 ScFv are described in U.S. Pat. No. 11,034,770 B2, the contents of which is incorporated herein by reference in its entirety, can also be used. The Cage and Key sequences are described in Lajoie, M. J. et al., Science. 2020; 369(6511): 1637-43, the contents of which is incorporated herein by reference in its entirety.

Synthesis and cloning of Cage and Key proteins: Codon optimized DNA sequences encoding the protein sequences of Table 2 were synthesized as fragments and cloned in mammalian expression vector pcDNA3.4. The resulting plasmids were amplified in E. coli (DH5a) cells and plasmid DNA was purified using Qiagen Maxi kit.

### Expression and Purification of Co-LOCKR Proteins

293 cells were transfected according to manufacturer protocol for MIRUS BIO™ TRANSIT™-293 Transfection Reagent (MIR 2700 Mirus Bio). 293 cells were plated in 10-cm tissue culture-treated dish 24 hours prior to transfection to achieve 80% confluency on the day of transfection. Maxi DNA of each Co-LOCKR plasmids were mixed with TRANSIT™-293 reagent and serum free media and incubated for 25 minutes at room temperature. Following incubation, the mixture was added dropwise to 293 cells. The cells were returned to the incubator for 72-96 hours. Finally, cell culture supernatant was collected, centrifuged to separate from any detached cells, and supplemented with phenylmethylsulfonyl fluoride (PMSF) to a final concentration of 1 mM to inhibit serine protease activity during purification. To purify Co-LOCKR proteins, 10 μL of TALON® Metal Affinity Resin (Takara Bio USA, San Jose, California) per 1 ml of supernatant was washed with PBST. Culture supernatant containing Co-LOCKR proteins was rotated overnight at 4° C. Next day, samples were centrifuged at 500 g for 5 minutes at 4° C., and supernatant was aspirated. Resin was resuspended in PBST, rotated for 10 minutes at 4° C., and centrifuged as done previously. This washing process was repeated for a total of 3 washes. 300 μL of elution buffer, 150 mM imidazole in PBS, per 100 μL of resin was added to the resin bed, and samples rotated at least 1 hour at 4° C. Samples were centrifuged at 500 g for 5 minutes at 4° C., and gently resuspended by pipetting before transfer to MICRO BIO-SPIN™ chromatography columns (Bio-Rad Laboratories, Hercules, California). Columns were placed in microcentrifuge tubes, and centrifuged at 10,000 g for 1 minute at 4° C. Eluates from TALON® resin were diluted to a final volume of 6 mL in PBS, and added to 30 kDa MWCO protein concentrator (ThermoFisher). Samples were centrifuged at 3,000 g for 15 minutes. Flow-through was discarded from the lower chamber of the concentrator, and concentrated samples were diluted in 5 mL of PBS before centrifugation under the conditions described above. Concentrated samples containing His-purified experimental constructs were collected, and either used immediately in subsequent experiments or frozen until use at −20° C.

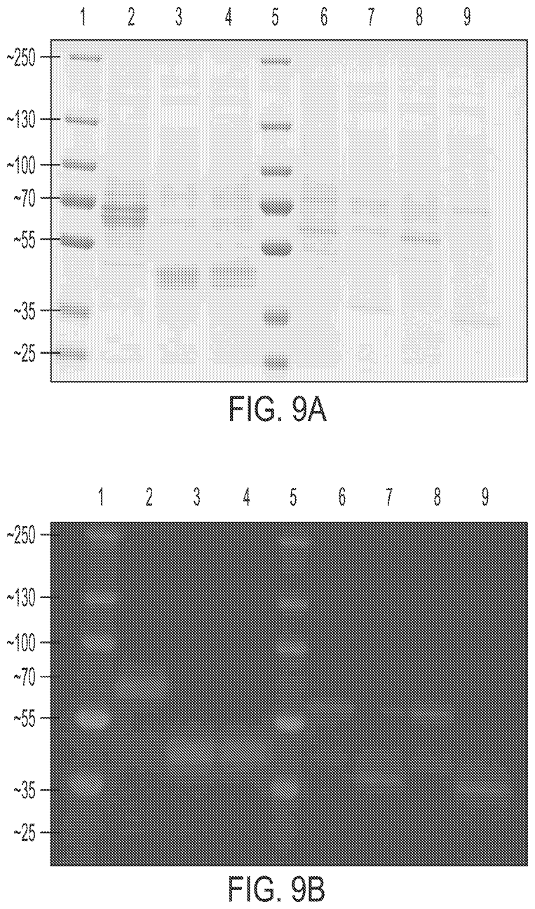

Staining and Immunoblot analysis: Co-LOCKR protein eluates were mixed with 2×-Laemmli sample buffer (Bio-Rad) containing β-mercaptoethanol. The mixture was heated at 95° C. for 10 minutes and 25 μL of sample was resolved on a Novex 4-20% tris-glycine mini gel (Invitrogen, Waltham, Massachusetts). To check expression and purity, the gel was stained with Coomassie G-250 stain (FIG. 9A). The gel was washed with deionized water for 5 min and stained with Coomassie G-250 for 20 min followed by a destaining for 10 min. The gel was photographed using LiCOR imaging system. For immunoblot analysis, proteins from the gel were transferred to a nylon membrane, blocked using 5% milk prepared in PBST, and incubated with anti-His6 primary antibodies in 0.5% milk prepared in PBST overnight. Following overnight incubation, the membrane is washed in PBST for a total of 4 washes. Membranes were probed with anti-mouse 800CW secondary antibody obtained from LI-COR Biosciences (Lincoln, Nebraska) for 30 minutes at room temperature. The secondary antibody was discarded and the membranes were washed 4 times as described above. The membranes were analyzed with Odyssey XF Imaging System (LI-COR) using ImageStudio software (LI-COR) (FIG. 9B).

TABLE 3
Key and Cage Proteins

>mIgk_His6_TEV_Key_EpCAM-ScFV (VL bold; VH underlined)
METDTLLLWVLLLWVPGSTGDGSHHHHHHGSGSENLYFQGSGGSDEARKAIARVKRESKRI
VEDAERLIREAAAASEKISREAERLIRGGGSGSGSGSGKPGQASGSELQMTQSPSSLSASV
GDRVTITCRASQSISSYLNWYQQKPGQPPKLLIYWASTRESGVPDRFSGSESGTNYTLTIS
SLQPEDFATYFCQQSDSLPITFGQGTRLDIQGSTSGSGKPGSGEGSGEVQLLESGGGVVQP
GRSLRLSCAASGFTFSSYGMHWVRQAPGKGLEWVAVISYDGSNKYYADSVKGRFTISRDNS
KNTLYLQMNSLRAEDTAVYYCAKDMGWGSGWRPYYYYGMDVWGQGTTVTVSSAPTKAPDVF
PL (SEQ ID NO. 14)
>mIgk_His6_TEV_Key-N3_EpCAM-ScFV (VL bold; VH underlined)
METDTLLLWVLLLWVPGSTGDGSHHHHHHGSGSENLYFQGSGGSDEAIARVKRESKRIVED
AERLIREAAAASEKISREAERLIRGGGSGSGSGSGKPGQASGSELQMTQSPSSLSASVGDR
VTITCRASQSISSYLNWYQQKPGQPPKLLIYWASTRESGVPDRFSGSESGTNYTLTISSLQ
PEDFATYFCQQSDSLPITFGQGTRLDIQGSTSGSGKPGSGEGSGEVQLLESGGGVVQPGRS
LRLSCAASGFTFSSYGMHWVRQAPGKGLEWVAVISYDGSNKYYADSVKGRFTISRDNSKNT
LYLQMNSLRAEDTAVYYCAKDMGWGSGWRPYYYYGMDVWGQGTTVTVSSAPTKAPDVFPL
(SEQ ID NO. 15)
>mIgk_His6_TEV_EpCAM-ScFV_Cage (VL bold; VH underlined)
METDTLLLWVLLLWVPGSTGDGSHHHHHHGSGSENLYFQGSGGSELQMTQSPSSLSASVGD
RVTITCRASQSISSYLNWYQQKPGQPPKLLIYWASTRESGVPDRFSGSESGTNYTLTISSL
QPEDFATYFCQQSDSLPITFGQGTRLDIQGSTSGSGKPGSGEGSGEVQLLESGGGVVQPGR
SLRLSCAASGFTFSSYGMHWVRQAPGKGLEWVAVISYDGSNKYYADSVKGRFTISRDNSKN
TLYLQMNSLRAEDTAVYYCAKDMGWGSGWRPYYYYGMDVWGQGTTVTVSSAPTKAPDVFPL
SGSGSGKPGQASGSELARKLLEASTKLQRLNIRLAEALLEAIARLQELNLELVYLAVELTD
PKRIRDEIKEVKDKSKEIIRRAEKEIDDAAKESEKILEEAREAISGSGSELAKLLLKAIAE
TQDLNLRAAKAFLEAAAKLQELNIRAVELLVKLTDPATIREALEHAKRRSKEIIDEAERAI
RAAKRESERIIEEARRLIEKGSGSGSELARELLRAHAQLQRLNLELLRELLRALAQLQELN
LDLLRLASELTDEIWIAQELRRIGDEFNAYYADAERLIREAAAASEKISREAERLIR
(SEQ ID NO. 16)
>mIgk_His6_TEV_EpCAM-ScFV_Cage-I287A (VL bold; VH underlined)
METDTLLLWVLLLWVPGSTGDGSHHHHHHGSGSENLYFQGSGGSELQMTQSPSSLSASVGD
RVTITCRASQSISSYLNWYQQKPGQPPKLLIYWASTRESGVPDRFSGSESGTNYTLTISSL
QPEDFATYFCQQSDSLPITFGQGTRLDIQGSTSGSGKPGSGEGSGEVQLLESGGGVVQPGR
SLRLSCAASGFTFSSYGMHWVRQAPGKGLEWVAVISYDGSNKYYADSVKGRFTISRDNSKN
TLYLQMNSLRAEDTAVYYCAKDMGWGSGWRPYYYYGMDVWGQGTTVTVSSAPTKAPDVFPL
SGSGSGKPGQASGSELARKLLEASTKLQRLNIRLAEALLEAIARLQELNLELVYLAVELTD
PKRIRDEIKEVKDKSKEIIRRAEKEIDDAAKESEKILEEAREAISGSGSELAKLLLKAIAE
TQDLNLRAAKAFLEAAAKLQELNIRAVELLVKLTDPATIREALEHAKRRSKEIIDEAERAI
RAAKRESERIIEEARRLIEKGSGSGSELARELLRAHAQLQRLNLELLRELLRALAQLQELN
LDLLRLASELTDEIWIAQELRRIGDEFNAYYADAERLIREAAAASEKISREAERLAR
(SEQ ID NO. 17)
>mIgk_His6_TEV_Key_CD163-ScFV (VL bold; VH underlined)
METDTLLLWVLLLWVPGSTGDGSHHHHHHGSGSENLYFQGSGGSDEARKAIARVKRESKRI
VEDAERLIREAAAASEKISREAERLIRGGGSGSGSGSGKPGQASGSQVQLQESGPGLVKPS
ETLSLTCTVSGYSITSDYAWNWIRQFPGNKLEWMGYITYSGSTYYNPSLKSRVTISVDTSK
NQFSLKLSSVTAADTATYYCVSGTYYFDYWGQGTTLTVSSGSTSGSGKPGSGEGSGDIVMT
QSPSSLSASVGDRVTITCRASQSVSSDVAWFQQKPGKSPKPLIYYASNRYSGVPSRFSGSG
SGTDFTLTISSLQAEDFAVYFCGQDYTSPRTFGGGTKLEIKR (SEQ ID NO. 18)
>mIgk_His6_TEV_Key-N3_CD163-ScFV (VL bold; VH underlined)
METDTLLLWVLLLWVPGSTGDGSHHHHHHGSGSENLYFQGSGGSDEAIARVKRESKRIVED
AERLIREAAAASEKISREAERLIRGGGSGSGSGSGKPGQASGSQVQLQESGPGLVKPSETL
SLTCTVSGYSITSDYAWNWIRQFPGNKLEWMGYITYSGSTYYNPSLKSRVTISVDTSKNQF
SLKLSSVTAADTATYYCVSGTYYFDYWGQGTTLTVSSGSTSGSGKPGSGEGSGDIVMTQSP
SSLSASVGDRVTITCRASQSVSSDVAWFQQKPGKSPKPLIYYASNRYSGVPSRFSGSGSGT
DFTLTISSLQAEDFAVYFCGQDYTSPRTFGGGTKLEIKR (SEQ ID NO. 19)
>mIgk_His6_TEV_CD163-ScFV_Cage (VL bold; VH underlined)
METDTLLLWVLLLWVPGSTGDGSHHHHHHGSGSENLYFQGSGGSQVQLQESGPGLVKPSET
LSLTCTVSGYSITSDYAWNWIRQFPGNKLEWMGYITYSGSTYYNPSLKSRVTISVDTSKNQ
FSLKLSSVTAADTATYYCVSGTYYFDYWGQGTTLTVSSGSTSGSGKPGSGEGSGDIVMTQS
PSSLSASVGDRVTITCRASQSVSSDVAWFQQKPGKSPKPLIYYASNRYSGVPSRFSGSGSG
TDFTLTISSLQAEDFAVYFCGQDYTSPRTFGGGTKLEIKRSGSGSGKPGQASGSELARKLL
EASTKLQRLNIRLAEALLEAIARLQELNLELVYLAVELTDPKRIRDEIKEVKDKSKEIIRR
AEKEIDDAAKESEKILEEAREAISGSGSELAKLLLKAIAETQDLNLRAAKAFLEAAAKLQE
LNIRAVELLVKLTDPATIREALEHAKRRSKEIIDEAERAIRAAKRESERIIEEARRLIEKG
SGSGSELARELLRAHAQLQRLNLELLRELLRALAQLQELNLDLLRLASELTDEIWIAQELR
RIGDEFNAYYADAERLIREAAAASEKISREAERLIR (SEQ ID NO. 20)
>mIgk_His6_TEV_CD163-ScFV_Cage-I287A (VL bold; VH underlined)
METDTLLLWVLLLWVPGSTGDGSHHHHHHGSGSENLYFQGSGGSQVQLQESGPGLVKPSET
LSLTCTVSGYSITSDYAWNWIRQFPGNKLEWMGYITYSGSTYYNPSLKSRVTISVDTSKNQ
FSLKLSSVTAADTATYYCVSGTYYFDYWGQGTTLTVSSGSTSGSGKPGSGEGSGDIVMTQS
PSSLSASVGDRVTITCRASQSVSSDVAWFQQKPGKSPKPLIYYASNRYSGVPSRFSGSGSG
TDFTLTISSLQAEDFAVYFCGQDYTSPRTFGGGTKLEIKRSGSGSGKPGQASGSELARKLL
EASTKLQRLNIRLAEALLEAIARLQELNLELVYLAVELTDPKRIRDEIKEVKDKSKEIIRR
AEKEIDDAAKESEKILEEAREAISGSGSELAKLLLKAIAETQDLNLRAAKAFLEAAAKLQE
LNIRAVELLVKLTDPATIREALEHAKRRSKEIIDEAERAIRAAKRESERIIEEARRLIEKG
SGSGSELARELLRAHAQLQRLNLELLRELLRALAQLQELNLDLLRLASELTDEIWIAQELR
RIGDEFNAYYADAERLIREAAAASEKISREAERLAR (SEQ ID NO. 21)

### Example 3—Generation of Split-CAR-T

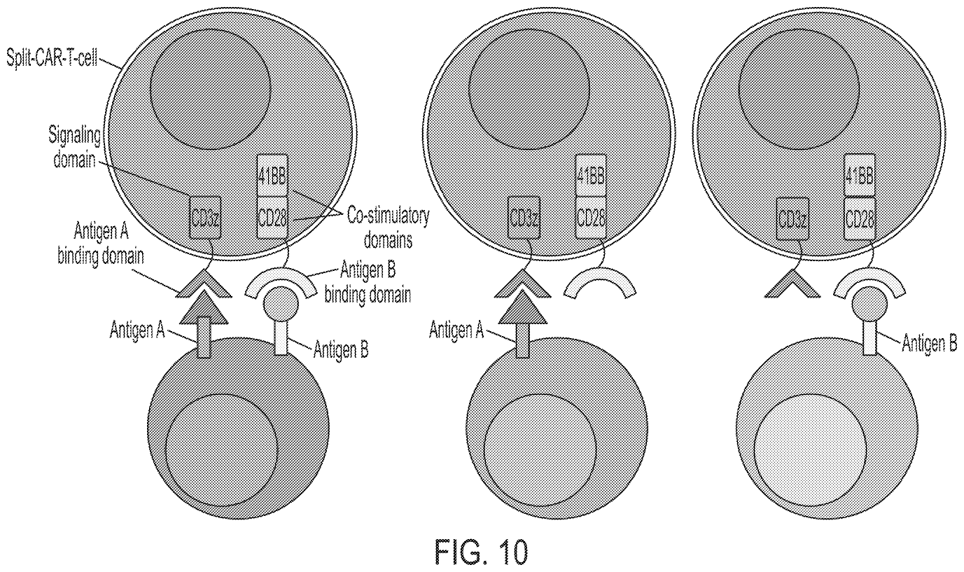

In a split chimeric antigen receptor T cell (Split-CAR-T) system, two modules, one with a CAR and other with chimeric costimulatory receptor (CCR), are expressed in the same T-cell to achieve balanced signaling and maximal T-cell cytotoxic activity on a target cell expressing two different targeted antigens (FIG. 10) (21). The CAR module comprises an antigen binding domain targeting one antigen and CD3z signaling domain. The CCR module contains an antigen binding domain targeting another antigen and two or more co-stimulatory domains.

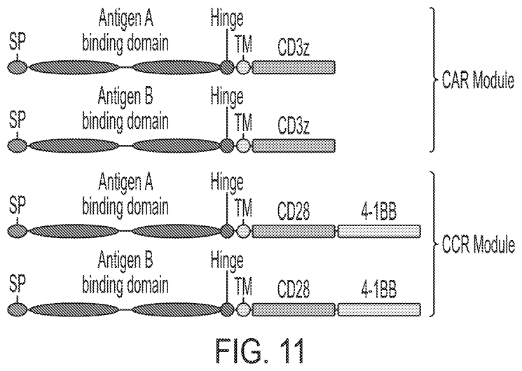

Design: Schematic design of EpCAM and CD163 targeting CAR and CCR modules is shown in FIG. 11. In some embodiments, the CAR module was designed to include: 1) a signal peptide for membrane targeting (derived from GM-CSF or CD8 alpha), 2) ScFv sequences (derived from either anti-EpCAM or anti-CD163 antibodies), 3) a hinge region (derived from CD8), 4) a transmembrane domain (derived from CD8), and 5) a CD3z signaling domain. In some embodiments, the CCR module was designed to include: 1) a signal peptide for membrane targeting (derived from GM-CSF), 2) ScFv sequences (derived from either anti-EpCAM or anti-CD163 antibodies), 3) a hinge region (derived from CD28), 4) a transmembrane domain (derived from CD28), and 5) a CD28 co-stimulatory domain, and 6) a 4-1BB co-stimulatory domain. For the antigen binding domain binding to EpCAM, we selected anti-EpCAM ScFv: the variable light chain (VL) and variable heavy chain (VH) sequences of anti-EpCAM ScFv have been described in U.S. Pat. No. 7,632,925 B2, the contents of each of which is incorporated herein by reference in its entirety. For the antigen binding domain binding to CD163, we selected anti-CD163 ScFv: the variable light chain (VL) and variable heavy chain (VH) sequences of anti-CD163 ScFv have been described in U.S. Pat. No. 9,724,426 B2 and U.S. Pat. No. 11,034,770 B2, the contents of each of which is incorporated herein by reference in their entirety. The sequences of various Split-CAR modules are described in Table 4.

Synthesis and cloning of Split-CAR modules: Codon optimized DNA sequences encoding the protein sequences of Table 4 were synthesized as fragments and cloned in pHIV-dTomato or pHIV-eGFP lentiviral vector backbone. The resulting plasmids were amplified in E. coli (DH5a) cells and plasmid DNA was purified using Qiagen Maxi kit.

TABLE 4
Split-CAR modules

>anti-EpCAM_CD28_41BB (Human antibody from phage library; HD70) (VL bold; VH
underlined)
MLLLVTSLLLCELPHPAFLLIPELQMTQSPSSLSASVGDRVTITCRASQSISSYLNWYQQK
PGQPPKLLIYWASTRESGVPDRFSGSESGTNYTLTISSLQPEDFATYFCQQSDSLPITFGQ
GTRLDIQGSTSGSGKPGSGEGSGEVQLLESGGGVVQPGRSLRLSCAASGFTFSSYGMHWVR
QAPGKGLEWVAVISYDGSNKYYADSVKGRFTISRDNSKNTLYLQMNSLRAEDTAVYYCAKD
MGWGSGWRPYYYYGMDVWGQGTTVTVSSAPTKAPDVFPLLDNEKSNGTIIHVKGKHLCPSP
LFPGPSKPFWVLVVVGGVLACYSLLVTVAFIIFWVRSKRSRLLHSDYMNMTPRRPGPTRKH
YQPYAPPRDFAAYRSKRGRKKLLYIFKQPFMRPVQTTQEEDGCSCRFPEEEEGGCEL
(SEQ ID NO. 22)
>anti-CD163 CD8_CD3z (Humanized antibody from mouse clone Mac-158) (VL bold;
VH underlined)
MLLLVTSLLLCELPHPAFLLIPQVQLQESGPGLVKPSETLSLTCTVSGYSITSDYAWNWIR
QFPGNKLEWMGYITYSGSTYYNPSLKSRVTISVDTSKNQFSLKLSSVTAADTATYYCVSGT
YYFDYWGQGTTLTVSSGSTSGSGKPGSGEGSGDIVMTQSPSSLSASVGDRVTITCRASQSV
SSDVAWFQQKPGKSPKPLIYYASNRYSGVPSRFSGSGSGTDFTLTISSLQAEDFAVYFCGQ
DYTSPRTFGGGTKLEIKRALSNSIMYFSHFVPVFLPAKPTTTPAPRPPTPAPTIASQPLSL
RPEASRPAAGGAVHTRGLDFACDIYIWAPLAGTCGVLLLSLVITRVKFSRSADAPAYQQGQ
NQLYNELNLGRREEYDVLDKRRGRDPEMGGKPRRKNPQEGLYNELQKDKMAEAYSEIGMKG
ERRRGKGHDGLYQGLSTATKDTYDALHMQALPPR (SEQ ID NO. 23)
>anti-CD163 CD8 CD3z (Humanized antibody from mouse clone Mac2-158) (VL bold;
VH underlined)
MALPVTALLLPLALLLHAARPQVQLQESGPGLVKPSETLSLTCTVSGYSITSDYAWNWIRQ
FPGNKLEWMGYITYSGSTYYNPSLKSRVTISVDTSKNQFSLKLSSVTAADTATYYCVSGTY
YFDYWGQGTTLTVSSGGGGSGGGGSGGGGSDIVMTQSPSSLSASVGDRVTITCRASQSVSS
DVAWFQQKPGKSPKPLIYYASNRYSGVPSRFSGSGSGTDFTLTISSLQAEDFAVYFCGQDY
TSPRTFGGGTKLEIKRALSNSIMYFSHFVPVFLPAKPTTTPAPRPPTPAPTIASQPLSLRP
EASRPAAGGAVHTRGLDFACDIYIWAPLAGTCGVLLLSLVITRVKFSRSADAPAYQQGQNQ
LYNELNLGRREEYDVLDKRRGRDPEMGGKPRRKNPQEGLYNELQKDKMAEAYSEIGMKGER
RRGKGHDGLYQGLSTATKDTYDALHMQALPPR (SEQ ID NO. 24)
>anti-CD163 CD8 CD3z (Mouse antibody from patent US11034770B2) (VL bold; VH
underlined)
MALPVTALLLPLALLLHAARPDIQMTQSPSSLSASVGDRVTITCRASQSISSYLNWYQQKP
GKAPKLLIYAASSLQSGVPSRFSGSGSGTDFTLTISSLQPEDFATYYCQQSYSTPRGTFGQ
GTKVEIKGGGGSGGGGSGGGGSEVQLVESGGGVVQPGRSLRLSCAASGFTFSSYAMHWVRQ
APGKGLEWVAVISYDGSNKYYADSVKGRFTISRDNSKNTLYLQMNSLRAEDTAVYYCAREN
VRPYYDFWSGYYSEYYYYGMDVWGQGTTVTVSSAALSNSIMYFSHFVPVFLPAKPTTTPAP
RPPTPAPTIASQPLSLRPEASRPAAGGAVHTRGLDFACDIYIWAPLAGTCGVLLLSLVITR
VKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR (SEQ ID
NO. 25)
>anti-CD163 CD8 CD3z (Humanized antibody from mouse clone Mac2-158) (VL bold;
VH underlined)
MALPVTALLLPLALLLHAARPQVQLQESGPGLVKPSETLSLTCTVSGYSITSDYAWNWIRQ
FPGNKLEWMGYITYSGSTYYNPSLKSRVTISVDTSKNQFSLKLSSVTAADTATYYCVSGTY
YFDYWGQGTTLTVSSGGGGSGGGGSGGGGSDIVMTQSPSSLSASVGDRVTITCRASQSVSS
DVAWFQQKPGKSPKPLIYYASNRYSGVPSRFSGSGSGTDFTLTISSLQAEDFAVYFCGQDY
TSPRTFGGGTKLEIKRDYKDDDDKALSNSIMYFSHFVPVFLPAKPTTTPAPRPPTPAPTIA
SQPLSLRPEASRPAAGGAVHTRGLDFACDIYIWAPLAGTCGVLLLSLVITRVKFSRSADAP
AYQQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR (SEQ ID NO. 26)

### REFERENCES FOR EXAMPLES 1-3

1. Gast C E, Silk A D, Zarour L, Riegler L, Burkhart J G, Gustafson K T, et al. Cell fusion potentiates tumor heterogeneity and reveals circulating hybrid cells that correlate with stage and survival. Sci Adv. 2018; 4(9):eaat7828.
2. Powell A E, Anderson E C, Davies P S, Silk A D, Pelz C, Impey S, et al. Fusion between Intestinal epithelial cells and macrophages in a cancer context results in nuclear reprogramming. Cancer Res. 2011; 71(4):1497-505.
3. Sutton T L, Walker B S, Wong M H. Circulating Hybrid Cells Join the Fray of Circulating Cellular Biomarkers. Cell Mol Gastroenterol Hepatol. 2019; 8(4):595-607.
4. Adams D L, Martin S S, Alpaugh R K, Charpentier M, Tsai S, Bergan R C, et al. Circulating giant macrophages as a potential biomarker of solid tumors. Proc Natl Acad Sci USA. 2014; 111(9):3514-9.
5. Tang C M, Zhu P, Li S, Makarova O V, Amstutz P T, Adams D L. Blood-based biopsies-clinical utility beyond circulating tumor cells. Cytometry A. 2018; 93(12):1246-50.
6. Adams D L, Adams D K, Alpaugh R K, Cristofanilli M, Martin S S, Chumsri S, et al. Circulating Cancer-Associated Macrophage-Like Cells Differentiate Malignant Breast Cancer and Benign Breast Conditions. Cancer Epidemiol Biomarkers Prev. 2016; 25(7):1037-42.
7. Aguirre L A, Montalban-Hernandez K, Avendano-Ortiz J, Marin E, Lozano R, Toledano V, et al. Tumor stem cells fuse with monocytes to form highly invasive tumor-hybrid cells. Oncoimmunology. 2020; 9(1):1773204.
8. Aljabery F, Olsson H, Gimm O, Jahnson S, Shabo I. M2-macrophage infiltration and macrophage traits of tumor cells in urinary bladder cancer. Urol Oncol. 2018; 36(4):159 e19-e26.
9. Augustyn A, Adams D L, He J, Qiao Y, Verma V, Liao Z, et al. Giant Circulating Cancer-Associated Macrophage-Like Cells Are Associated With Disease Recurrence and Survival in Non-Small-Cell Lung Cancer Treated With Chemoradiation and Atezolizumab. Clin Lung Cancer. 2021; 22(3):e451-e65.
10. Clawson G A, Matters G L, Xin P, Imamura-Kawasawa Y, Du Z, Thiboutot D M, et al. Macrophage-tumor cell fusions from peripheral blood of melanoma patients. PLoS One. 2015; 10(8):e0134320.
11. Clawson G A, Matters G L, Xin P, McGovern C, Wafula E, dePamphilis C, et al. “Stealth dissemination” of macrophage-tumor cell fusions cultured from blood of patients with pancreatic ductal adenocarcinoma. PLoS One. 2017; 12(9):e0184451.
12. Pawelek J M. Tumour-cell fusion as a source of myeloid traits in cancer. Lancet Oncol. 2005; 6(12):988-93.
13. Pawelek J M. Fusion of bone marrow-derived cells with cancer cells: metastasis as a secondary disease in cancer. Chin J Cancer. 2014; 33(3):133-9.
14. Shabo I, Olsson H, Sun X F, Svanvik J. Expression of the macrophage antigen CD163 in rectal cancer cells is associated with early local recurrence and reduced survival time. Int J Cancer. 2009; 125(8):1826-31.
15. Shabo I, Stal O, Olsson H, Dore S, Svanvik J. Breast cancer expression of CD163, a macrophage scavenger receptor, is related to early distant recurrence and reduced patient survival. Int J Cancer. 2008; 123(4):780-6.
16. Shabo I, Svanvik J. Expression of macrophage antigens by tumor cells. Adv Exp Med Biol. 2011; 714:141-50.
17. Shabo I, Svanvik J, Lindstrom A, Lechertier T, Trabulo S, Hulit J, et al. Roles of cell fusion, hybridization and polyploid cell formation in cancer metastasis. World J Clin Oncol. 2020; 11(3):121-35.
18. Sieler M, Weiler J, Dittmar T. Cell-Cell Fusion and the Roads to Novel Properties of Tumor Hybrid Cells. Cells. 2021; 10(6).
19. Walker B S, Sutton T L, Zarour L, Hunter J G, Wood S G, Tsikitis V L, et al. Circulating Hybrid Cells: A Novel Liquid Biomarker of Treatment Response in Gastrointestinal Cancers. Ann Surg Oncol. 2021; 28(13):8567-78.
20. Lajoie M J, Boyken S E, Salter A I, Bruffey J, Rajan A, Langan R A, et al. Designed protein logic to target cells with precise combinations of surface antigens. Science. 2020; 369(6511):1637-43.
21. Kloss C C, Condomines M, Cartellieri M, Bachmann M, Sadelain M. Combinatorial antigen recognition with balanced signaling promotes selective tumor eradication by engineered T cells. Nat Biotechnol. 2013; 31(1):71-5.

What is claimed is:

1. A bispecific molecule comprising at least two antigen binding regions, wherein each antigen binding region binds a different antigen on The First Cell (TFC) of a cancer.

2. The bispecific molecule of claim 1, wherein the first antigen is a marker of epithelial cell lineage.

3. The bispecific molecule of claim 2, wherein the marker of epithelial cell lineage is any one of the markers in FIG. 2.

4. The bispecific molecule of claim 2, wherein the marker of epithelial cell lineage is epithelial cellular adhesion molecule (EpCAM).

5. The bispecific molecule of claim 4, wherein EpCAM comprises SEQ ID NO: 7 or SEQ ID NO12.

6. The bispecific molecule of any one of claims 1-5, wherein the second antigen is a marker of macrophage cell lineage.

7. The bispecific molecule of claim 6, wherein the marker of macrophage cell lineage is any one of the markers in FIG. 1.

8. The bispecific molecule of claim 6, wherein the marker of macrophage cell lineage is CD163.

9. The bispecific molecule of claim 8, wherein CD163 comprises SEQ ID NO: 8, SEQ ID NO: 9, SEQ ID NO:10, SEQ ID NO: 11, or SEQ ID NO: 13.

10. The bispecific molecule of any of claims 1-9, wherein the cancer comprises a solid tumor.

11. The bispecific molecule of claim 1, wherein the cancer is breast cancer, brain cancer, gastrointestinal cancer, pancreatic cancer, kidney cancer, liver cancer, lung cancer, thymic carcinoma, ovarian cancer, prostate cancer, or endometrial cancer.

12. The bispecific molecule of claim 11, wherein the gastrointestinal cancer is stomach cancer or colorectal cancer.

13. The bispecific molecule of any one of claims 1-9, wherein the cancer comprises a liquid cancer.

14. The bispecific molecule of claim 13, wherein the liquid cancer is leukemia, lymphoma, or myeloma.

15. The bispecific molecule of claim 13, wherein the liquid cancer is acute myeloid leukemia (AML).

16. The bispecific molecule of claim 13, wherein the liquid cancer is B-cell malignancy.

17. The bispecific molecule of claim 13, wherein the liquid cancer is myeloid neoplasm, myelodysplastic syndrome (MDS), myeloproliferative neoplasm (MPN), MDS/MPN overlap syndrome, acute myeloid leukemia or chronic myeloid leukemia.

18. The bispecific molecule of any of claims 1-17, wherein the first antigen binding region comprises a light chain variable (VL) region and a heavy chain variable (VH) region, wherein the VL region comprises a light chain CDR1 (CDRL1) of SEQ ID NO: 36, a light chain CDR2 (CDRL2) of SEQ ID NO: 37, a light chain CDR3 (CDRL3) of SEQ ID NO: 38 and the VH region comprises a heavy chain CDR1 of SEQ ID NO: 33, a heavy chain CDR2 of SEQ ID NO: 34, and a heavy chain CDR3 of SEQ ID NO: 35.

19. The bispecific molecule of claim 18, wherein the second antigen binding region comprises a light chain variable (VL) region and a heavy chain variable (VH) region, wherein the VL region comprises a light chain CDR1 (CDRL1) of SEQ ID NO: 42, a light chain CDR2 (CDRL2) of SEQ ID NO: 43, a light chain CDR3 (CDRL3) of SEQ ID NO: 44 and the VH region comprises a heavy chain CDR1 of SEQ ID NO: 39, a heavy chain CDR2 of SEQ ID NO: 40, and a heavy chain CDR3 of SEQ ID NO: 41.

20. The bispecific molecule of claim 18, wherein the second antigen binding region comprises a light chain variable (VL) region and a heavy chain variable (VH) region, wherein the VL region comprises a light chain CDR1 (CDRL1) of SEQ ID NO: 48, a light chain CDR2 (CDRL2) of SEQ ID NO: 49, a light chain CDR3 (CDRL3) of SEQ ID NO: 50 and the VH region comprises a heavy chain CDR1 of SEQ ID NO: 45, a heavy chain CDR2 of SEQ ID NO: 46, and a heavy chain CDR3 of SEQ ID NO: 47.

21. The bispecific molecule of any of claims 1-17, wherein the first antigen binding region comprises a light chain variable (VL) region of SEQ ID NO: 28 and a heavy chain variable (VH) region of SEQ ID NO:27.

22. The bispecific molecule of claim 21, wherein the second antigen binding region comprises a light chain variable (VL) region of SEQ ID NO: 30 and a heavy chain variable (VH) region of SEQ ID NO: 29.

23. The bispecific molecule of claim 21, wherein the second antigen binding region comprises a light chain variable (VL) region of SEQ ID NO: 32 and a heavy chain variable (VH) region of SEQ ID NO: 31.

24. The bispecific molecule of claim 13-17, wherein the first antigen and the second antigen are markers of macrophage cell lineage.

25. The bispecific molecule of claim 24, wherein the first antigen is CD117, CD34, or CD123 and the second antigen is CD163.

26. The bispecific molecule of claim 1, wherein TFC is a metastatic TFC.

27. The bispecific molecule of any of claims 1-26, wherein the bispecific molecule is a bispecific antibody or antigen binding fragment thereof.

28. The bispecific molecule of claim 27, wherein the bispecific antibody is conjugated to drug.

29. The bispecific antibody of claim 28, wherein the drug is a toxin.

30. The bispecific antibody of claim 28, wherein the drug is a chemotherapy agent.

31. The bispecific molecule of any of claims 1-26, wherein the bispecific molecule comprises bi-nanobodies, BiTE, tandAbs, DARTs, DART-Fc, DARPin, scFv, scFv-HAS-scFV, and DNL-Fab3.

32. The bispecific molecule of any of claims 1-26, wherein the bispecific molecule is a bispecific chimeric antigen receptor (CAR).

33. The bispecific molecule of any of claims 1-26, wherein the bispecific molecule is a Co-LOCKR comprising a first polypeptide and a second polypeptide, wherein the first polypeptide and the second polypeptide each bind a different antigen on TFC of a cancer.

34. The bispecific molecule of claim 33, wherein the first polypeptide comprises SEQ ID NO: 14, SEQ ID NO: 15, SEQ ID NO: 18, or SEQ ID NO: 19.

35. The bispecific molecule of claim 33, wherein the second polypeptide comprises SEQ ID NO: 16, SEQ ID NO: 17, SEQ ID NO: 20, or SEQ ID NO: 21.

36. The bispecific molecule of claim 32, wherein the bispecific CAR is a synNotch CAR.

37. The bispecific molecule of claim 1-26, wherein the bispecific molecule is a Co-LOCKR comprising a first polypeptide, a second polypeptide, and a third polypeptide, wherein the first polypeptide and the second polypeptide each bind a different antigen on TFC of a cancer, wherein the third polypeptide binds to a CAR, and wherein the third polypeptide is operably linked to the first polypeptide or the second polypeptide.

38. The bispecific molecule of claim 1-26, wherein the bispecific molecule comprises a split-CAR-T system comprising a chimeric antigen receptor (CAR) module and a chimeric costimulatory receptor (CCR) module,
wherein the CAR module comprises a polypeptide comprising a first antigen binding region and CD3z signaling domain,
wherein the CCR module comprises a polypeptide comprising a second antigen binding region and two or more co-stimulatory domains, and
wherein the CAR module and the CCR module each bind a different antigen on TFC of a cancer.

39. The bispecific molecule of claim 28, wherein the split-CAR-T system comprises one or more of the polypeptide sequences in Table 4.

40. A pharmaceutical composition comprising a bispecific molecule of any of claims 1-39.

41. A polynucleotide encoding a bispecific molecule of any of claims 1-39.

42. A vector comprising a polynucleotide of claim 41.

43. A virus comprising a polynucleotide of claim 41.

44. A genetically engineered cell comprising a bispecific molecule of any of claims 1-39.

45. A genetically engineered cell comprising a polynucleotide of claim 41.

46. A method of treating or preventing cancer in a subject in need thereof, the method comprising administering to the subject a bispecific molecule comprising at least two antigen binding regions, wherein each antigen binding region binds a different antigen on The First Cell (TFC) of a cancer.

47. The method of claim 46, wherein the first antigen is a marker of epithelial cell lineage.

48. The method of claim 47, wherein the marker of epithelial cell lineage is any one of the markers in FIG. 2.

49. The method of claim 48, wherein the marker of epithelial cell lineage is epithelial cellular adhesion molecule (EpCAM).

50. The method of claim 49, wherein EpCAM comprises SEQ ID NO: 7 or SEQ ID NO: 12.

51. The method of any one of claims 46-50, wherein the second antigen is a marker of macrophage cell lineage.

52. The method of claim 51, wherein the marker of macrophage cell lineage is any one of the markers in FIG. 1.

53. The method of claim 51, wherein the marker of macrophage cell lineage is CD163.

54. The method of claim 53, wherein CD163 comprises SEQ ID NO: 8, SEQ ID NO: 9, SEQ ID NO: 10, SEQ ID NO: 11, or SEQ ID NO: 13.

55. The method of any one of claims 46-54, wherein the cancer comprises a solid tumor.

56. The method of claim 55, wherein the cancer is breast cancer, brain tumor, gastrointestinal, pancreatic cancer, kidney cancer, liver cancer, lung cancer, thymic carcinoma, ovarian cancer, prostate cancer, or endometrial cancer.

57. The method of claim 56, wherein the gastrointestinal cancer is stomach cancer or colorectal cancer.

58. The method of claim 46-54, wherein the cancer is a liquid cancer.

59. The method of claim 58, wherein the liquid cancer is leukemia, lymphoma, or myeloma.

60. The method of claim 58, wherein the liquid cancer is acute myeloid leukemia (AML).

61. The method of claim 58, wherein the liquid cancer is B-cell malignancy.

62. The method of claim 58, wherein the liquid cancer is myeloid neoplasm, myelodysplastic syndrome (MDS), myeloproliferative neoplasm (MPN), MDS/MPN overlap syndrome, acute myeloid leukemia or chronic myeloid leukemia.

63. The method of any of claims 46-62, wherein the first antigen binding region comprises a light chain variable (VL) region and a heavy chain variable (VH) region, wherein the VL region comprises a light chain CDR1 (CDRL1) of SEQ ID NO: 36, a light chain CDR2 (CDRL2) of SEQ ID NO: 37, a light chain CDR3 (CDRL3) of SEQ ID NO: 38 and the VH region comprises a heavy chain CDR1 of SEQ ID NO: 33, a heavy chain CDR2 of SEQ ID NO: 34, and a heavy chain CDR3 of SEQ ID NO: 35.

64. The method of claim 63, wherein the second antigen binding region comprises a light chain variable (VL) region and a heavy chain variable (VH) region, wherein the VL region comprises a light chain CDR1 (CDRL1) of SEQ ID NO: 42, a light chain CDR2 (CDRL2) of SEQ ID NO: 43, a light chain CDR3 (CDRL3) of SEQ ID NO: 44 and the VH region comprises a heavy chain CDR1 of SEQ ID NO: 39, a heavy chain CDR2 of SEQ ID NO: 40, and a heavy chain CDR3 of SEQ ID NO: 41.

65. The method of claim 63, wherein the second antigen binding region comprises a light chain variable (VL) region and a heavy chain variable (VH) region, wherein the VL region comprises a light chain CDR1 (CDRL1) of SEQ ID NO: 48, a light chain CDR2 (CDRL2) of SEQ ID NO: 49, a light chain CDR3 (CDRL3) of SEQ ID NO: 50 and the VH region comprises a heavy chain CDR1 of SEQ ID NO: 45, a heavy chain CDR2 of SEQ ID NO: 46, and a heavy chain CDR3 of SEQ ID NO: 47.

66. The method of any of claims 46-62, wherein the first antigen binding region comprises a light chain variable (VL) region of SEQ ID NO: 28 and a heavy chain variable (VH) region of SEQ ID NO:27.

67. The method of claim 66, wherein the second antigen binding region comprises a light chain variable (VL) region of SEQ ID NO: 30 and a heavy chain variable (VH) region of SEQ ID NO: 29.

68. The method of claim 66, wherein the second antigen binding region comprises a light chain variable (VL) region of SEQ ID NO: 32 and a heavy chain variable (VH) region of SEQ ID NO: 31.

69. The method of claim 58-62, wherein the first antigen and the second antigen are markers of macrophage cell lineage.

70. The method of claim 69, wherein the first antigen is CD117, CD34, or CD123 and the second antigen is CD163.

71. The method of claim 46, wherein TFC is a metastatic TFC.

72. The method of any one of claims 46-71, wherein the bispecific molecule is a bispecific antibody or antigen binding fragment thereof.

73. The method of claim 72, wherein the bispecific antibody is conjugated to a drug.

74. The method of claim 73, wherein the drug is a toxin.

75. The method of claim 73, wherein the drug is a chemotherapy agent.

76. The method of any one of claims 46-71, wherein the bispecific molecule comprises bi-nanobodies, BiTE, tandAbs, DARTs, DART-Fc, DARPin, scFv, scFv-HAS-scFV, and DNL-Fab3.

77. The method if any one of claims 46-71, wherein the bispecific molecule is a bispecific chimeric antigen receptor (CAR).

78. The method of any one of claims 46-71, wherein the bispecific molecule is a Co-LOCKR comprising a first polypeptide and a second polypeptide, wherein the first polypeptide and the second polypeptide each bind a different antigen on TFC of a cancer.

79. The method of claim 66, wherein the first polypeptide comprises SEQ ID NO: 14, SEQ ID NO: 15, SEQ ID NO: 18, or SEQ ID NO: 19.

80. The method of claim 66, wherein the second polypeptide comprises SEQ ID NO: 16, SEQ ID NO: 17, SEQ ID NO: 20, or SEQ ID NO: 21.

81. The method of claim 80, wherein the bispecific CAR is a synNotch CAR.

82. The method of any one of claim 80, wherein the bispecific molecule is a Co-LOCKR comprising a first polypeptide, a second polypeptide, and a third polypeptide, wherein the first polypeptide and the second polypeptide each bind a different antigen on TFC of a cancer, wherein the third polypeptide binds to the bispecific CAR, and wherein the third polypeptide is operably linked to the first polypeptide or the second polypeptide.

83. The method of claim 46-71, wherein the bispecific molecule comprises a split-CAR-T system comprising a chimeric antigen receptor (CAR) module and a chimeric costimulatory receptor (CCR) module,
wherein the CAR module comprises a polypeptide comprising a first antigen binding region and CD3z signaling domain,
wherein the CCR module comprises a polypeptide comprising a second antigen binding region and two or more co-stimulatory domains, and
wherein the CAR module and the CCR module each bind a different antigen on TFC of a cancer.

84. The method of claim 63, wherein the split-CAR-T system comprises one or more of the polypeptide sequences in Table 4.

85. An engineered cell expressing at least one marker of epithelial cell lineage and at least one marker of macrophage cell lineage.

86. The cell of claim 85, wherein the at least one marker of epithelial cell lineage is any one of the markers in FIG. 2.

87. The cell of claim 85, wherein the at least one marker of epithelial cell lineage is epithelial cellular adhesion molecule (EpCAM).

88. The cell of claim 87, wherein EpCAM comprises SEQ ID NO: 7 or SEQ ID NO: 12.

89. The cell of any one of claims 85-88, wherein the at least one marker of macrophage cell lineage is any one of the markers in FIG. 1.

90. The cell of claim 85, wherein the at least one marker of macrophage cell lineage is CD163.

91. The cell of claim 90, wherein CD163 comprises SEQ ID NO: 8, SEQ ID NO: 9, SEQ ID NO: 10, SEQ ID NO: 11, or SEQ ID NO: 13.

92. A method of diagnosing cancer, wherein the method comprises detecting a cell expressing at least one marker of epithelial cell lineage and at least one marker of macrophage cell lineage.

93. The method of claim 92, wherein the at least one marker of epithelial cell lineage is any one of the markers in FIG. 2.

94. The method of claim 92, wherein the at least one marker of epithelial cell lineage is epithelial cellular adhesion molecule (EpCAM).

95. The method of claim 94, wherein EpCAM comprises SEQ ID NO: 7.

96. The method of any one of claims 92-95, wherein the at least one marker of macrophage cell lineage is any one of the markers in FIG. 1.

97. The method of claim 96, wherein the at least one marker of macrophage cell lineage is CD163.

98. The method of claim 97, wherein CD163 comprises SEQ ID NO: 8, SEQ ID NO: 9, SEQ ID NO: 10, or SEQ ID NO: 11.

99. The method of claim 92, wherein the detecting comprises an assay wherein a bispecific molecule binds to the at least one marker of epithelial cell lineage and the at least one marker of macrophage cell lineage.

100. The method of any of claims 92-99, wherein the cancer comprises a solid tumor.

101. The method of claim 100, wherein the cancer is breast cancer, brain cancer, gastrointestinal cancer, pancreatic cancer, kidney cancer, liver cancer, lung cancer, thymic carcinoma, ovarian cancer, prostate cancer, or endometrial cancer.

102. The method of claim 101, wherein the gastrointestinal cancer is stomach cancer or colorectal cancer.

103. The method of any one of claims 92-99, wherein the cancer is a liquid cancer.

104. The method of claim 103, wherein the liquid cancer is leukemia, lymphoma, or myeloma.

105. The method of claim 103, wherein the liquid cancer is acute myeloid leukemia (AML).

106. The method of claim 103, wherein the liquid cancer is B-cell malignancy.

107. The method of claim 103, wherein the liquid cancer is myeloid neoplasm, myelodysplastic syndrome (MDS), myeloproliferative neoplasm (MPN), MDS/MPN overlap syndrome, acute myeloid leukemia or chronic myeloid leukemia.
